(19) United States
(12) Patent Application Publication
(10) Pub. No.: US 2024/0390327 A1
(43) Pub. Date: Nov. 28, 2024

(54) THERAPEUTIC COMPOSITIONS AND METHODS

(71) Applicant: Johns Hopkins University, Baltimore, MD (US)

(72) Inventors: Shenandoah Robinson, Owing Mills, MD (US); Lauren Jantzie, Baltimore, MD (US)

(73) Assignee: Johns Hopkins University, Baltimore, MD (US)

(21) Appl. No.: 18/278,998

(22) PCT Filed: Feb. 26, 2022
(86) PCT No.: PCT/US22/18046

§ 371 Date: Aug. 25, 2023

Related U.S. Application Data
(60) Provisional application No. 63/154,638, filed on Feb. 26, 2021.

(51) Int. Cl. A61K 31/4045; A61K 31/472; A61K 38/18; A61P 25/00; A61P 25/14; A61P 25/28

(52) U.S. Cl. CPC A61K 31/4045; A61K 31/472; A61K 38/1816; A61P 25/00; A61P 25/14; A61P 25/28

(57) ABSTRACT

In one aspect, methods and compositions are provided for treating a subject suffering from or susceptible to prenatal opioid exposure. In a further aspect, methods and compositions are provided for treating a subject suffering from or susceptible traumatic brain injury, intracranial hemorrhage, acquired hydrocephalus, demyelinating diseases including multiple sclerosis, neurological symptoms from systemic lupus erythematosus, and neurodegenerative diseases. In an additional aspect, methods and compositions are provided for treating subject having a COVID-19 infection, exhibiting symptoms of a COVID-19 infection, having suspected exposure to COVID-19, or suspected of suffering from long COVID or post-acute sequelae of COVID-19 (PASC).

The present application claims the benefit of U.S. provisional application 63/154,638 filed Feb. 26, 2021, which is incorporated herein by reference in its entirety.

This invention was made with government support under DA052402 awarded by the National Institutes of Health and under W81XWH-18-1-0166 and W81XWH-18-1-0167 awarded by Army Medical Research and Materiel Command. The government has certain rights in the invention.

### BACKGROUND

Illicit drug use during pregnancy is a worldwide problem. Maternal opiate dependency during pregnancy has been managed by methadone and buprenorphine (Suboxone) maintenance therapy to reduce illicit drug use and thus improve perinatal outcomes.

The fetus is exposed to methadone and other opioids such as morphine, fentanyl, codeine and heroin which all can readily cross the placenta. The main adverse effect of maternal methadone treatment and/or opioid consumption during pregnancy is neonatal withdrawal, or Neonatal Abstinence Syndrome (NAS). Withdrawal has been reported to occur in more than 50% of infants of substance using mothers. The presentation of NAS signified by central and autonomic nervous system, respiratory and gastrointestinal signs and symptoms occurs in the first days of life and frequently requires lengthy treatment of up to several months.

### SUMMARY

In a first aspect, we now provide methods and compositions for treating a subject suffering from or susceptible to prenatal opioid exposure and including for the treatment of brain injury associated with in utero and perinatal opioid exposure.

The present methods and compositions include treatment of neonatal abstinence syndrome (NAS) and neonatal opioid withdrawal syndrome (NOWS), and the long-term sequelae including white matter loss and cognitive dysfunction.

The methods and compositions further include treatment of perinatal opioid exposure, including for the treatment of brain injury associated with in utero and perinatal opioid exposure.

The methods and compositions also include treatment of substance use disorder (particularly opioid abuse), including substance use disorder in pregnant women and those receiving medication assisted treatment (MAT) in pregnancy for substance use disorder perinatal opioid exposure.

The methods and composition further include treatment of brain injury associated with in utero and perinatal opioid exposure.

The methods and compositions further include treatment of perinatal brain injuries, including cerebral palsy, perinatal traumatic brain injury and acquired forms of hydrocephalus including posthemorrhagic hydrocephalus, postinfectious hydrocephalus, and post-traumatic hydrocephalus.

In a first embodiment of this first aspect, the methods and compositions include administration to subject in need thereof an effective amount of melatonin agent.

In a second embodiment of this first aspect, the methods and compositions include administration to subject in need thereof an effective amount of an erythropoietin compound.

In a third embodiment of this first aspect, the methods and compositions include a therapy (preferably, a combination therapy) that comprises:
i) a melatonin agent; and
ii) an erythropoietin compound.

In a fourth embodiment of this first aspect, the methods and compositions include administration to subject in need thereof an effective amount of a prolyl hydroxylase domain inhibitor (PHD inhibitor) compound.

In a fifth embodiment of this first aspect, the methods and compositions include a therapy (preferably, a combination therapy) that comprises:
i) a melatonin agent; and
ii) prolyl hydroxylase domain inhibitor (PHD inhibitor) compound and/or an erythropoietin compound.

In a second aspect, methods and compositions are provided for treating a subject suffering from or susceptible traumatic brain injury, intracranial hemorrhage, acquired hydrocephalus, demyelinating diseases including multiple sclerosis, neurological symptoms from systemic lupus erythematosus and other autoimmune disorders, post-infectious neuro-inflammatory disorders, and neurodegenerative diseases.

The methods and compositions may be used to treat adult brain insults including hydrocephalus, brain injury (including TBI) and other neurodegenerative insults.

In this second aspect, the methods and compositions include a therapy (preferably, a combination therapy) that comprises:
i) a melatonin agent; and
ii) one or more prolyl hydroxylase domain inhibitor ((PHD inhibitor) compounds

In a third aspect, methods and compositions are provided for treating subject having a COVID-19 infection, exhibiting symptoms of a COVID-19 infection, or having suspected exposure to COVID-19, or suffering sequelae from SARS-CoV-2, currently referred to as long COVID or post-acute sequelae of COVID-19 (PASC).

Long COVID or post-acute sequelae of COVID-19 (PASC) is a condition marked in one aspect by the continuation of COVID-19 symptoms or the emergence of new ones after recovery from the acute or initial phase of COVID-19. In one aspect, a subject may be assessed and identified as suffering from long COVID or post-acute sequelae of COVID-19 (PASC) where the subject exhibits the persistence of COVID-19 symptoms for four weeks or longer after the initial onset or diagnosis of a COVID-19 infection.

Long COVID or post-acute sequelae of COVID-19 (PASC) also has been characterized into the following two groups: (1) subacute or ongoing symptomatic SARS-CoV infection, which includes symptoms and abnormalities present from 4-12 weeks beyond acute COVID-19 or coronavirus infection; and (2) chronic or post-COVID-19 syndrome or coronavirus infection, which includes symptoms and abnormalities persisting or present beyond 12 weeks of the onset of acute COVID-19 or coronavirus infection and not attributable to alternative diagnoses. The present compostions and methods can be used to treat subjects suffering from either of such categories or groups of long COVID or post-acute sequelae of COVID-19 (PASC).

In a further embodiment of this aspect, methods and compositions are provided for treating cells infected by or exposed to COVID-19, including mammalian cells, particularly human cells.

In this third aspect, the methods and compositions include a therapy (preferably, a combination therapy) that comprises:
i) a melatonin agent; and
ii) one or more prolyl hydroxylase domain inhibitor (PHD inhibitor) compounds.

Pharmaceutical compostions are also provided comprising one or more therapeutic agents as disclosed herein. The compositions suitably may comprise one or more pharmaceutically acceptable carriers. In certain preferred aspects, the composition may be adapted for oral administration as a tablet or capsule.

Kits are also provided for use to treat or prevent a condition or disease as disclosed herein. Kits of the invention suitably may comprise 1) one or more compounds of therapeutic agents as disclosed herein; and 2) instructions for using the one or more compounds for treating or preventing a condition or disease as disclosed herein. Preferably, a kit will comprise a therapeutically effective amount of the one or more therapeutic compounds. The instructions suitably may be in written form, including as a product label.

Other aspects of the invention are disclosed infra

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows an exemplary in vivo model including neonatal EPO+MLT treatment.

FIG. 2 (includes FIGS. 2A-2I) shows that methadone Significantly Impairs Cognitive Function in Adult Rats Following Perinatal Exposure. Osmotic minipumps were implanted in pregnant rats on E16. Pups were born and in adulthood were tested on a touchscreen cognitive assessment. All rats exposed to saline and methadone were able to pass the visual discrimination (VD) task (A). However, those exposed to methadone committed more discrimination errors (B) and required more testing sessions (C) in order to achieve passing criteria. After achieving VD criterion, reversal learning was assessed. Adult rats exposed to methadone during the perinatal period had diminished ability to reverse the previously learned association (D). Methadone animals made significantly more incorrect responses (E) across the reversal paradigm, and also required more sessions to achieve passing criterion (F). In order to determine whether poor performance during reversal was associated with deficits in cognitive control or learning acquisition, we assessed the number of correction trials (G) required during the initial perseverative phase (accuracy <50%) and later learning phase (accuracy ≥50%). Methadone rats committed significantly more correction errors both during the perseverative phase H) and during the later learning phase (I) compared to saline control animals. Together, these data indicate that adult rats exposed to perinatal methadone are impaired in both early and late reversal learning, consistent with widespread learning and executive control dysfunction. *p<0.05, n=10-11/group.

FIG. 3 shows that methadone Reduces White Matter Fractional Anisotropy. Osmotic minipumps were implanted in pregnant rats on E16. Pups were born and underwent diffusion tensor imaging as adults. Compared to sham, methadone pups have reduced fractional anisotropy (FA) in major white matter tracts including the corpus callosum, internal and external capsule (arrows). Notably, treatment with EPO+MLT attenuates loss of FA in white matter and cortex. These data are consistent with EPO+MLT's ability to preserve white matter microstructure, axon and myelin integrity. Summary image registration of n=4-5/group.

FIG. 4 shows in vivo functional connectivity. Seed-based fcMRI (green crosshairs) reveals loss of connectivity after preterm injury. A thalamic voxel of interest was visualized using AFNI InstaCorr. Colored voxels indicate correlation at the network level with the thalamic seed region of interest at a level of p<0.05 uncorrected. Red color indicates positive correlation with thalamic region of interest. Blue indicates negative correlation.

FIG. 5 shows that EPO+MLT attenuates perinatal methadone-induced increases in neonatal serum inflammatory biomarkers. Osmotic minipumps were implanted in pregnant rats E16 to commence in utero methadone exposure. Pups were born at term and serum collected on P15. Using a translational MECI platform, biomarkers were assayed. Pups exposed to methadone commencing in utero had significantly increased IL-1b, TNFa, IL-6 and CXCL1, compared to controls. These data reflect significant and persistent increases in molecules central to ongoing inflammation, immune cell activation, and recruitment of monocytes and neutrophils. Significantly, EPO administered in the neonatal period (on postnatal day (P)1-5), together with MLT (P1-P10) reduced increases in these pro-inflammatory cytokines and chemokines consistent with mitigation of a diverse inflammatory response syndrome. *p<0.05, **p<0.01, ***p<0.001, n=5-6/group. Two-Way ANOVA with Bonferroni Correction.

FIG. 6 shows that opioids induce greater pro-inflammatory cytokine secretion by pbmcs compared to lps, a phenomenon blocked by EPO+MLT. PBMCs on postnatal day 21 (P21) were cultured and stimulated with control (media+vehicle), lipopolysaccharide (media+LPS) or buprenorphine (media+buprenorphine). The PBMCs secretome was then assayed after 3 h in culture for inflammatory biomarkers. Secreted levels of interferon gamma and IL-6 were significantly increased in PBMCs treated with buprenorphine compared to LPS. Notably, treatment of PBMCs with EPO+MLT (10 U/mL and 20 μM, respectively) or naloxone (10 μM), a mixed TLR4 and opioid antagonist, blocked the secretion of these proinflammatory mediators. Together these data indicate opioids induce immune cell modulation, a phenomenon that can be partially blocked by EPO+MLT. n=4,*p<0.05, **p<0.01, ***p<0.001. Two-Way ANOVA with Bonferroni Correction.

FIG. 7 (includes FIGS. 7A-7H) shows prenatal chorioamnionitis (CAM) plus postnatal day 1 (P1) intraventricular injection of lysed red blood cells (IVH), or CAM-IVH, causes progressive macrocephaly, ventriculomegaly and neurodevelopmental delay. (A) Experimental paradigm. (B) Coronal head ultrasound on P2 demonstrating bilateral IVH. (C) A representative P7 rat with CAM-IVH exhibits a domed cranial vault, while a sham-IVH rat does not. (D) CAM-IVH rats show a disproportionate increase in intra-aural distance (IAD), a surrogate for head circumference, while control rats (sham-veh, sham-IVH and CAM-veh) do not. (E) Hematoxylin and eosin staining of coronal sections of the lateral ventricle at P5 demonstrate ventriculomegaly in CAM-IVH rats. Bar=500 microns. (F) Ventricular volume measured with unbiased stereology of HE sections at P5 is mildly increased in CAM-veh rats, and markedly elevated in CAM-IVH rats, compared to sham-veh or sham-IVH rats. (G) Ventricular volume quantified from coronal cx vivo T2 MRI slices shows that ventriculomegaly in CAM-IVH rats is sustained through P21, compared to sham-veh, sham-IVH and CAM-veh rats. (H) Both CAM-veh and CAM-IVH rats exhibit delayed performance on cliff aversion, a neonatal neurodevelopmental test. (D is mixed model repeated measures ANOVA with Bonferroni's correction and all other comparisons are two-way ANOVA with Bonferroni's correction, *p<0.05, **p<0.01, ***p<0.001.)

FIG. 8 (includes FIGS. 8A-8D) shows that extended neonatal treatment with erythropoietin (EPO) plus melatonin (MLT) prevents progressive macrocephaly and neurodevelopmental delay, and reduces sustained ventriculomegaly in CAM-IVH rats. (A) At P16, a vehicle-treated CAM-IVH rat exhibits a domed cranial vault, while the EPO+MLT-treated CAM-IVH rat does not. (B) IADs of EPO+MLT-treated CAM-IVH rats diverge from those of vehicle-treated CAM-IVH rats, and become similar to sham-veh rats. (C) EPO+MLT treatment of CAM-IVH rats prevents delayed performance on cliff aversion, compared to vehicle-treated CAM-IVH rats. (D) Ventricular volume measured on MRI at P21 was defined as normal, and mild, moderate and severe ventriculomegaly. Sham-veh and sham-IVH rats all had normal size ventricles, while all vehicle-treated CAM-IVH rats had ventriculomegaly. After neonatal EPO+MLT treatment, 40% of CAM-IVH rats had normal size ventricles, and the proportion with more severe ventriculomegaly shifted to less severe. Importantly, none of the EPO+MLT CAM-IVH rats with residual ventriculomegaly had macrocephaly. (B: a mixed model repeated measures ANOVA with Bonferroni's correction; C: two-way ANOVA with Bonferroni's correction; D: Wilcoxon rank sum test, *p<0.05, **p≤0.01, ***p≤0.001.)

FIG. 9 shows coronal T2 images that demonstrate ventricular size. Directionally encoded color maps of coronal images reveal widespread white and gray matter microstructural abnormalities in vehicle-treated CAM-IVH rats that are prevented by neonatal EPO+MLT treatment. Color maps show loss of microstructural coherence and impaired directional diffusion in regions with poor microstructural integrity. Directional colors are red for transverse, green for vertical and blue for orthogonal to the plane.

FIG. 10 (includes FIGS. 10A-10F) shows diffusion tensor imaging (DTI) regional analyses and fractional anisotropy (FA) graphs quantify prevention of loss of microstructural integrity. (A) Diagram of DTI regions of interest (purple—striatum, yellow—corpus callosum, pink—external capsule white matter (ECWM), green—thalamus, and maroon—hippocampus). (B) Reduction of FA in the ECWM of vehicle-treated CAM-IVH rats is prevented in CAM-IVH rats with neonatal EPO+MLT treatment. (C) Similarly, reduction of FA in the central corpus callosum of vehicle-treated CAM-IVH rats is prevented by EPO+MLT treatment. (D) FA in the hippocampus is also decreased in vehicle-treated CAM-IVH rats compared to shams, and EPO+MLT treatment mitigates the damage. (E) Likewise, FA in the striatum is reduced in vehicle-treated CAM-IVH rats, and prevented in EPO+MLT-treated CAM-IVH rats. (F) FA in the thalamus is also lower in vehicle-treated CAM-IVH rats compared to shams, and normalizes with EPO+MLT treatment (all comparisons are two-way ANOVA with Bonferroni's correction, *p<0.05, **p<0.01, ***p<0.001).

FIG. 11 (includes FIGS. 11A-11E) shows that directional diffusivity in central corpus callosum and external capsule white matter is altered by CAM-IVH and EPO+MLT treatment. (A) Mean diffusivity is elevated in the corpus callosum vehicle-treated CAM-IVH rats, compared to shams and EPO+MLT-treated CAM-IVH rats. (B) Ellipsoids comprised of the three major eigenvectors of diffusion in the central corpus callosum illustrate the changes in vehicle-treated and EPO+MLT-treated CAM-IVH rats. (C) Axial diffusivity is relatively unaffected by CAM-IVH. (D) Vehicle-treated CAM-IVH rats exhibit elevated radial diffusivity (RD) in central corpus callosum, and neonatal EPO+MLT treatment normalizes the microstructural integrity. (E) Similarly, vehicle-treated CAM-IVH rats show increased RD in the ECWM, and neonatal EPO+MLT treatment normalizes the RD (all comparisons are two-way ANOVA with Bonferroni's correction, *p<0.05, **p<0.01, ***p<0.001).

FIG. 12 (includes FIGS. 12A-12C) shows scanning electron microscopy (SEM) images of P21 ependymal motile cilia that show preservation of cilia following EPO+MLT treatment in CAM-IVH rats. (A) Low magnification images demonstrate a sheet of tuft-like morphology in sham-veh rats that is disrupted in vehicle-treated CAM-IVH rats. CAM-IVH rats with EPO+MLT treatment exhibit preservation of the sheet of tufts of motile cilia. Bar=10 microns. (B) Higher magnification images reveal some motile cilia in vehicle-treated CAM-IVH rats are missing. Bar=10 microns. (C) High magnification images show the residual cilia appear bloated and matted in vehicle-treated rats, unlike the thinner, more upright cilia organized in tufts in sham-veh rats or EPO+MLT-treated CAM-IVH rats. Bar=2 microns.

FIG. 13 (includes FIGS. 13A-13C) shows that neonatal EPO+MLT treatment in CAM-IVH rats improves periventricular yes-associated protein (YAP) mRNA levels and reduces GFAP-expression. (A) At P15, periventricular YAP mRNA levels are reduced in micro-dissected ependyma from vehicle-treated CAM-IVH rats, compared to shams. This loss is partially, but significantly, prevented in CAM-IVH rats following EPO+MLT treatment. (B) GFAP-immunolabeling of coronal sections of the lateral ventricle show the choroid plexus appears shrunken in the large ventricle. EPO-MLT-treatment prevents this appearance of the choroid plexus. (C) Minimal GFAP-immunolabeling is present in the normal caliber third ventricle of a sham rat, while marked GFAP expression is present in the ependymal lining of the dilated third ventricle in a vehicle-treated CAM-IVH rat. In an EPO+MLT-treated CAM-IVH rat with mild ventriculomegaly, there is less GFAP present, and this pattern is more apparent in the EPO+MLT-treated CAM-IVH rat with a normal sized third ventricle (Bars=100 microns, two-way ANOVA with Bonferroni's correction, *p<0.05, ***p<0.001).

FIG. 14 (includes FIGS. 14A-14I) shows that CAM-IVH rats exhibited progressive macrocephaly and ventriculomegaly.

FIG. 15 (includes FIGS. 15A-15P) shows assessment neonatal neurodevelopment, a battery of age-appropriate, validated, functional tests.

FIG. 16 (includes FIGS. 16A-16G) shows results for whether the combination of EPO+MLT had any unanticipated impact on neurodevelopment.

FIG. 17 (includes FIGS. 17A-17D) shows open field testing of locomotion and disinhibition. (A) After prenatal injury, vehicle-treated rats are more mobile than shams, particularly in the last 5 min interval. Erythropoietin (EPO)+melatonin (MLT), but not EPO alone, normalizes the activity. (B) Rats typically avoid open areas. Vehicle-treated adult rats following prenatal injury spend more time immobile in the center zone than sham rats or EPO+MLT-treated rats. (C) Sham and EPO+MLT-treated injured rats spent more time in the peripheral zone than vehicle-treated or EPO-treated injured rats. (D) Similarly, vehicle-treated injured rats spent more time in the neutral zone than sham or EPO+MLT-treated rats. EPO treatment by itself did not improve disinhibition (two-way ANOVA with Bonferroni correction, *p<0.05, **p<0.01, ***p<0.001).

FIG. 18 (includes FIGS. 18A-18C) shows that prenatal injury impairs gait performance. (A) After prenatal injury, vehicle-treated rats contact their hindpaws with less area than sham or erythropoietin (EPO)+melatonin (MLT)-treated injured rats. (B) Similarly, vehicle-treated injured rats contact hindpaws with less pressure than shams or EPO+MLT-treated rats. (C) The percent of shared stance is elevated in vehicle-treated rats with prenatal injury compared to shams, and EPO+MLT treatment normalizes gait and posture (two-way ANOVA with Bonferroni correction, *p<0.05, **p<0.01, ***p<0.001).

FIG. 19 shows that prenatal injury impairs social interaction. After prenatal injury, pairs of vehicle-treated rats of the same sex but different litters have fewer social interactions than pairs of shams, or pairs of erythropoietin (EPO)+melatonin (MLT)-treated injured rats (two-way ANOVA with Bonferroni correction, *p<0.05, **p<0.01).

FIG. 20 (includes FIGS. 20A-20F) shows that touchscreen operant chamber testing reveals impaired cognition. (A) Fewer vehicle-treated rats after prenatal injury successfully reached passing criterion for visual discrimination, compared to shams or erythropoietin (EPO)+melatonin (MLT)-treated injured rats (*p<0.05). (B) Shams, vehicle-treated rats with prenatal injury, and EPO+MLT-treated rats with prenatal injury all committed approximately the same number of errors during visual discrimination testing. (c) The reaction latency between the three groups did not differ. (D) Likewise, the magazine latency was similar for all three groups. (E) Significantly fewer vehicle-treated rats after prenatal injury were able to pass criterion for VD and reversal (*p<0.05). (F) After prenatal injury, vehicle-treated rats required more correction trials in reversal learning paradigms than shams.

FIG. 21 shows spiral chest CT scan indicating multilobular patchy and ground-glass opacities in favor of COVID-19. CT scan, computed tomography scan.

FIG. 22 shows molecular pathways associated with EPO signaling within the cell. EPO, erythropoietin; EPO-R, erythropoietin receptor.

FIG. 23A shows a proposed hypothesis of the treatment based on SARS-CoV-2 illness case.

FIG. 23B shows exemplary model of post-COVID-like (PCL) syndrome, the rodent model of long COVID or PASC (post-acute sequelae of COVID-19) in humans.

FIG. 24A shows dose response curve (mechanical allodynia) and FIG. 24B shows dose response curve (thermal hypersensitivity).

FIG. 25 shows dose response curve with subacute nociception on P80 and 25 days post viral insult.

FIG. 26 shows an exemplary treatment model for middle age Post-COVID-like (PCL) Syndrome in rodents, PASC in humans.

FIG. 27 shows P60 acute results (females, nociception, baseline 5 days after viral insult).

FIG. 28 shows P60 acute results (males) from subacute nociception of FIG. 26.

FIG. 29A shows P60 acute results (open field, baseline 5 days after viral). FIG. 29B shows P60 acute results (females) and FIG. 29C shows P60 acute results (males).

FIG. 30 shows P80 subacute results (nociception, 25 days after viral illness).

FIG. 31 shows P80 acute results (females) and FIG. 32 shows P80 acute results (males) from subacute nociception of FIG. 30.

FIG. 33 shows chronic nociception 2 months after viral illness.

FIG. 34 shows an exemplary middle age Post-COVID-like (PCL) syndrome rat model of human PASC, also known as long COVID.

FIG. 35 shows that Post-COVID Like (PCL) illness yields deficits in cognition.

FIG. 36A-36B show that Post-COVID Like (PCL) illness yields a variable acute inflammatory secretome.

FIG. 37A-37B show that Post-COVID Like (PCL) illness yields a variable chronic inflammatory secretome.

FIGS. 38A-38C show exemplary rationale for combination treatment of PHD inhibitor and melatonin.

FIG. 39 shows an exemplary hypothesis of synergistic neural cell repair.

FIG. 40 shows an exemplary experimental design and development of treatment for PCL syndrome.

FIG. 41 shows that subacute treatment with ROX+MLT attenuates pain in a Post-Covid Like (PCL) model of chronic neurological sequelae (nociception).

FIG. 42 shows that subacute treatment with adaptaquin+MLT attenuates pain in a Post-Covid Like (PCL) model of chronic neurological sequelae (nociception).

FIG. 43 shows that subacute treatment with adaptaquin+MLT attenuates pain in a Post-Covid Like (PCL) model of chronic neurological sequelae (rat, male).

FIG. 44 shows that subacute treatment with adaptaquin+MLT attenuates pain in a Post-Covid Like (PCL) model of chronic neurological sequelae (rat, female).

FIG. 45 shows that treatment with ROX+MLT attenuates chronic pain in a Post-COVID like (PCL) model.

FIG. 46 shows that treatment with ADQ+MLT attenuates chronic pain in a Post-COVID like (PCL) model.

FIG. 47 shows that subacute treatment with ROX+MLT alleviates deficits of cognition in a Post-COVID like (PCL) model of chronic neurological sequelae.

FIGS. 48 and 49 show Roxadustat+Melatonin: 5 Choice Serial Reaction Time Test and results of Example 12 which follows.

FIG. 50 shows results of Roxadustat+Melatonin induction and maintenance regimen and results of Example 13 which follows.

FIG. 51 shows Diffusion Tensor Imaging—ROX+MLT Attenuates White Matter Loss Secondary to PCL and results of Example 14 which follows.

FIG. 52 shows functional activation in fMRI in PCL model and results of Example 15 which follows.

FIG. 53 depicts results showing treatment improves memory in posthemorrhagic hydrocephalus and results of Example 16 which follows.

FIG. 54 depicts results Exploratory Behavior in Post-traumatic Hydrocephalus and results of Example 17 which follows.

FIG. 55 (includes FIGS. 55A-C) depicts results showing EPO+MLT Treatment Normalizes Post-traumatic Hydrocephalus and results of Example 18 which follows.

FIG. 56 depicts results showing Erythropoietin (EPO)+melatonin (MLT) treatment resolve deficits in cilia function and cerebral spinal fluid (CSF) flow in posthemorrhagic hydrocephalus of prematurity (PHHP) and results of Example 19 which follows.

FIG. 57 depicts results showing EPO+MLT Rescues Ependymal Motile Cilia Impairment and results of Example 20 which follows.

FIG. 58 depicts results showing EPO+MLT Normalizes T cells in Posthemorrhagic Hydrocephalus (PHH) and results of Example 21 which follows.

FIG. 59 (includes FIGS. 59A and B) depicts results showing EPO+MLT Normalizes Serum Biomarkers in Post-traumatic Hydrocephalus (PTH) and results of Example 22 which follows.

FIG. 60 depicts results showing EPO+MLT Treatment Improves White Matter Integrity in Post-traumatic Hydrocephalus (PTH) and results of Example 23 which follows.

FIG. 61 depicts results showing EPO+MLT Treatment Improves White Matter Integrity in Post-traumatic Hydrocephalus (PTH) and results of Example 24 which follows.

FIG. 62 (includes FIGS. 62A and 62B) depicts results showing ROX+MLT Attenuates Macrocephaly and Increased Intracranial Pressure (ICP) in Existing Posthemorrhagic Hydrocephalus of Prematurity (PHHP) and results of Example 25 which follows.

FIG. 63 (includes FIGS. 63A-63C) depicts results showing ROX+MLT Attenuates Increased Intracranial Pressure and Gait in Existing Posthemorrhagic Hydrocephalus of Prematurity (PHHP) and results of Example 26 which follows.

FIG. 64 (includes FIGS. 64A-64D) depicts results showing EPO+MLT Normalizes Gait Metrics in a Model of Cerebral Palsy Secondary to Preterm Brain Injury and results of Example 27 which follows.

FIG. 65 (includes FIGS. 65A-65B) depicts results showing neonatal EPO+MLT administered normalizes functional activation cerebral palsy in vivo study and results of Example 28 which follows.

FIG. 66 (includes FIGS. 66A-66B) depicts results showing Roxadustat (ROX) and Melatonin (MLT) treatment attenuates nociception induced by mechanical and thermal stimuli in adult rats with preterm brain injury and results of Example 29 which follows.

FIG. 67 (includes FIGS. 67A-67B) depicts results showing EPO+MLT Mitigates Deficits in Inhibition Observed on an Open Field Test in Adult Animals Prenatally Exposed to Methadone and results of Example 30 which follows.

FIG. 68 (includes FIGS. 68A-68B) depicts results showing EPO+MLT Attenuates Hyperactivity in Adult Rats Exposed to Methadone In Utero and results of Example 31 which follows.

FIG. 69 depicts results showing EPO+MLT Mitigates Specific Gait Abnormalities In Adult Rats Exposed to Methadone In Utero and results of Example 32 which follows.

FIG. 70 (includes FIGS. 70A-70B) depicts results showing EPO+MLT Mitigates Allodynia and Thermal Hypersensitivity in Adult Rats Exposed to Methadone In Utero and results of Example 33 which follows.

FIG. 71 depicts results showing EPO+MLT Attenuates Blood Biomarkers of Inflammation in Adult Rats with Prenatal Methadone Exposure and results of Example 34 which follows.

FIG. 72 depicts results showing EPO+MLT Ameliorates Deficits in Structural Connectivity in Adult Rats Exposed to Methadone In Utero and results of Example 35 which follows.

FIG. 73 depicts results showing EPO+MLT Ameliorates Deficits in Structural Connectivity in Adult Rats Exposed to Methadone In Utero and results of Example 36 which follows.

FIG. 74 (includes FIGS. 74A-74B) depicts results showing EPO+MLT Ameliorates Deficits in Fractional Anisotropy (FA) in Adult Rats After Methadone Exposure and results of Example 37 which follows.

FIG. 75 depicts results showing EPO+MLT Normalizes Functional Activation in the Striatum of Adult Animals after Exposure to Methadone In Utero and results of Example 38 which follows.

FIG. 76 depicts results showing EPO+MLT Normalizes Functional Activation in the Amygdala of Adult Animals after Exposure to Methadone In Utero and results of Example 39 which follows.

### DETAILED DESCRIPTION

### Melatonin and Melatonin Agent

A melatonin agent as referred to herein includes melatonin (N-acetyl-5-methoxytryptamine) and related compounds including melatonin receptor agonists such as TAK-375, agomelatine, LY 156735, CGP 52608, low-dose melatonin A, GR196429, S20242, S23478, S24268, S25150, BMS-214778, melatonin receptor research compound A, GW290569, controlled release melatonin, luzindole, GR135531, melatonin agonist A, melatonin analogue B, melatonin agonist C, melatonin agonist D, melatonin agonist E, melatonin agonist F, melatonin agonist G, melatonin agonist H, melatonin agonist I, melatonin analog J, melatonin analog K, melatonin analog L, AH-001, GG-012, enol-3-IPA, ML-23, SL-18.1616, IP-100-9, melatonin low-dose B, sleep inducing peptide A, oros-melatonin, AH-017, AH-002, and IP-101. Such compounds are also described in U.S. 2005/0164987.

A preferred melatonin of the present methods, compostions and kits is melatonin (N-acetyl-5-methoxytryptamine) and salts thereof.

A melatonin agent or melatonin (N-acetyl-5-methoxytryptamine) is sometimes referred to herein as MLT.

### Erythropoietin Compound

An erythropoietin compound as referred to herein includes erythropoietin and in particular human recombinant erythropoietin as well as other agents use for erythropoietin activity such as new erythropoiesis stimulating protein (NESP); the protein conjugate synthetic erythropoiesis protein (SEP) (contains monodisperse, negatively charged polymers instead of oligosaccharides); and various EPO-mimetics which may be capable of acting as EPO in dimerizing the EPO receptor and include peptides and nonpeptides.

An erythropoietin compound or erythropoietin is sometimes referred to herein as EPO.

Various erythropoietin compounds that may be used in the present methods, compostions and kits including Epoetin alpha (Epogen, Eprex, Procrit); Epoetin beta (Recormon); Epoetin omega (Epomax, Hemax); Epoetin beta (NeoRecormon); Darbepoetin alkfa (Aranesp); Epoetin delta (Dynepo); methoxy polymethylene glycol epotein beta (Mircera); carbamylated EPO (CEPO); biosimilars of Epoetein alpha (Binocrit, Abseamed, Epoetein Alfa Hexal); Epoetein zeta (Retacrit, Silapo); and Epoetin theta (Biopoin, Eporatio); and ARA290.

EPO-mimetics also may be used as an erythropoietin compounds in the present methods, compostions and kits including those agents disclosed in US2012/0082669; Qureshi et al. Proc Nat Acad Sci USA, 1999 Oct. 12:96(21):12156-61 (non-peptide mimics).

### Prolyl Hydroxylase Domain Inhibitor (PHD Inhibitor) Compounds

A prolyl hydroxylase domain inhibitor (PHD inhibitor) compound as referred to herein can be readily identified including through in vitro and in vivo assays such as disclosed for instance in U.S. Pat. No. 9,708,296 (including Example that includes assessment of inhibition PHD enzyme and optional secondary assay Cell-Based HIF-Alpha Stabilization Assay; and Example C of in vivo cardioprotection assay).

Specific suitable prolyl hydroxylase domain inhibitor (PHD inhibitor) compounds for use in the present methods, compositions and kits include JTZ-951; FG4592 (Roxadustat) from FibroGen, GSK1278863 (Daprodustat) from GlaxoSmithKline, Bay85-3934 (Molidustat) from Bayer; AKB-6548 (Vadadustat) from Akebia; adaptaquin; TM6008 (6-amino-1, 3-di methyl-5-(2-pyridin-2-yl-quinoline-4-carbonyl)-1H-pyrimidine-2,4-dione) and TM6089 is 6-amino-1,3-di-methyl-5-[2-(pyridin-2-ylsulfanyl)-acetyl]-1H-pyrimidine-2,4-dione (see Nangaku et al. Arteriosclerosis, Thrombosis, and Vascular Biology, 27(12), 2548-2554 (December 2007); and compounds disclosed in US2010/035906, US2010/0093803 and U.S. Pat. No. 9,708,296.

### EPO and Melatonin (MLT)

We predict EPO+MLT will normalize the microenvironment and mitigate molecular and cellular markers of inflammation. Without being bound by any theory, we believe that in utero opioid exposure is defined by excess inflammatory signaling. We predict this inflammatory state reprograms and primes immune cells as well as being toxic to neural cells. Using state-of-the-art preclinical magnetic resonance imaging (MRI), including diffusion tensor (DTI)4-6,26,28-30 and functional connectivity (FcMRI), in concert with translational touchscreen cognitive assessment 5,26,27 we will rigorously test structure-function relationships secondary to perinatal opioid exposure and potential repair/recovery with EPO+MLT treatment. We will focus on neural networks and major white matter tracts essential to cognition and executive function. A clinical biomarker platform, multiplex electrochemiluminescent immunoassay (MECI), will be used to assay proinflammatory cytokines and chemokines over the lifespan and in response to EPO+MLT treatment. We predict that peripheral immune cells are primed and reprogrammed following opioid exposure favoring a pro-inflammatory secretome and that EPO+MLT will attenuate peripheral inflammatory network signatures, and associated cognitive deficits and imaging abnormalities.

A combination therapy is sometimes referred to the following exemplary format with a “+” symbol between the multiple agents being use in combination: EPO+MLT.

There is urgent need to define the full spectrum of adverse infant and childhood outcomes associated with prenatal opioid exposure.1 Although Neonatal Opioid Withdrawal Syndrome (NOWS) is a well-recognized consequence of prenatal opioid exposure, the full lifelong impact of exposure on the developing brain is unknown.1,31,32 Notably, the negative effects of perinatal opioid exposure (POE) extend far beyond initial hospital admissions for withdrawal symptoms and the associate diagnoses that are apparent in the first days or weeks of life.1 Thus, to address the public health crisis that is stemming from the multitude of infants with in utero opioid exposure who are growing up with altered neurodevelopmental trajectories, we propose a rigorous, mechanistically driven, neuroreparative therapeutic strategy of erythropoietin (EPO) plus melatonin (MLT) to treat the long-term sequelae of POE, including neurocognitive impairment. POE includes the use and misuse of prescription opioids, such as oxycodone, morphine, codeine, illicit opioids (e.g., heroin), and exposure to medications used to manage opioid use disorder, such as methadone and buprenorphine.33-35 The effects and the consequences of untreated opioid use disorder and substance misuse in pregnancy are well defined,31 and include gaps in prenatal care, preterm birth, growth restriction, abruptio placentae, low birth weight, respiratory depression, neonatal withdrawal, and neonatal death.31,36,37 Maternal mortality reviews in several states also identify substance use as a major risk factor for pregnancy-associated death.31,38 Accordingly, the safety of opioids during early pregnancy has been evaluated in several observational studies. They have focused on fetal death, neural tube defects, congenital malformations and gestational length as primary outcome measures, with no study of postnatal sequela. 31,39-43 This is significant because clinical investigations show that neonates exposed to opioids in utero have smaller brains, and reduced basal ganglia and cerebellar volumes.44 Notably, prenatal opioid exposure is associated with microstructural brain injury seen on high-resolution MRI and impaired neurodevelopment. 45 Larger clinical investigations show children born to women who have been prescribed opioids, including methadone, are at higher risk of neurodevelopmental impairment,45,46 with lower Mental Development Index and Psychomotor Development Index scores than unexposed children.45,46These children also have microstructural alterations in major white matter tracts that are present at birth and can be longitudinally visualized throughout childhood. 45-47

There is urgent need to define the full spectrum of adverse infant and childhood outcomes associated with prenatal opioid exposure.1 Although Neonatal Opioid Withdrawal Syndrome (NOWS) is a well-recognized consequence of prenatal opioid exposure, the full lifelong impact of exposure on the developing brain is unknown.1,31,32 Notably, the negative effects of perinatal opioid exposure (POE) extend far beyond initial hospital admissions for withdrawal symptoms and the associate diagnoses that are apparent in the first days or weeks of life.1 Thus, to address the public health crisis that is stemming from the multitude of infants with in utero opioid exposure who are growing up with altered neurodevelopmental trajectories, we propose a rigorous, mechanistically driven, neuroreparative therapeutic strategy of erythropoietin (EPO) plus melatonin (MLT) to treat the long-term sequelae of POE, including neurocognitive impairment. POE includes the use and misuse of prescription opioids, such as oxycodone, morphine, codeine, illicit opioids (e.g., heroin), and exposure to medications used to manage opioid use disorder, such as methadone and buprenorphine.33-35 The effects and the consequences of untreated opioid use disorder and substance misuse in pregnancy are well defined,31 and include gaps in prenatal care, preterm birth, growth restriction, abruptio placentae, low birth weight, respiratory depression, neonatal withdrawal, and neonatal death.31,36,37 Maternal mortality reviews in several states also identify substance use as a major risk factor for pregnancy-associated death.31,38 Accordingly, the safety of opioids during early pregnancy has been evaluated in several observational studies. They have focused on fetal death, neural tube defects, congenital malformations and gestational length as primary outcome measures, with no study of postnatal sequela. 31,39-43 This is significant because clinical investigations show that neonates exposed to opioids in utero have smaller brains, and reduced basal ganglia and cerebellar volumes.44 Notably, prenatal opioid exposure is associated with microstructural brain injury seen on high-resolution MRI and impaired neurodevelopment. 45 Larger clinical investigations show children born to women who have been prescribed opioids, including methadone, are at higher risk of neurodevelopmental impairment,45,46 with lower Mental Development Index and Psychomotor Development Index scores than unexposed children.45,46These children also have microstructural alterations in major white matter tracts that are present at birth and can be longitudinally visualized throughout childhood. 45-47

To address the gap in knowledge of the principal mechanisms of neural injury and immune alterations induced by opioids early in development, we developed a model of perinatal opioid exposure that accurately incorporates the intact maternal-placental fetal axis (Animal Model Summary).27 Using this model, we recently published that perinatal opioid exposure shares many features of a profound neuroinflammatory disease. 27 Specifically, we found that perinatal methadone exposure increases inflammatory cytokines in the neonatal peripheral circulation, and reprograms and primes the immune system through sustained peripheral immune hyperreactivity (SPIHR). 27 We also discovered that in the brain, perinatal methadone exposure not only increases chemokines and cytokines throughout a crucial neurodevelopmental period, but also altered microglia morphology consistent with activation, and upregulated cerebral inflammatory gene networks. 27 This increase in neuroinflammation coincided with reduced myelin basic protein and altered neurofilament expression, as well as reduced structural coherence and significantly decreased fractional anisotropy on DTI. 27 This culminated in the adult rats exposed to methadone in the perinatal period having significant impairment in associative learning and executive control as assessed using touchscreen technology. 27 As these data revealed a distinct systemic and neuroinflammatory signature associated with perinatal methadone exposure, suggestive of an altered CNS microenvironment, and revealed imaging and cognitive findings consistent with clinical literature, we turned to our prior studies of neurorepair and novel therapeutic strategies. Erythropoietin (EPO) and melatonin (MLT) are endogenous, pluripotent cytokines that are essential for neurodevelopment. EPO and MLT have overlapping mechanisms of action in the developing brain, and are currently in separate neonatal clinical trials to test if they reduce chronic neurological deficits in preterm infants (NCT01207778, NCT02036073, NCT01378273, NCT04235673, NCT00649961, NCT03806816). 16,19,24 Thus, because EPO+MLT are endogenous developmentally-regulated neurorestorative agents with pluripotent mechanisms, including established anti-inflammatory properties, we propose to validate whether a combined, high dose, extended regimen can mitigate structural and functional neural injury secondary to perinatal opioid exposure.

### Definitions

As used herein, the terms below have the meanings indicated.

When ranges of values are disclosed, and the notation “from n₁ . . . to n₂” or “between n₁ . . . and n₂” is used, where n₁ and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Combination therapy” or “in combination” or other similar term means the administration of two or more therapeutic agents (particularly, a melatonin agent, a prolyl hydroxylase domain inhibitor (PHD inhibitor) compound and/or an erythropoietin compound) to treat a therapeutic condition or disorder described in the present disclosure. Such administration encompasses co-administration of two or more of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

Further, the term “combination therapy” or “in combination” in the context of the administration can also refer to the prophylactic use of a therapy to a subject when used with at least one additional therapy. The use of the term “combination therapy” or “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours or 24 hours or more before), concomitantly with, or subsequent to (e.g., 1 minute, 5 minutes, 15 minutes, 30 minutes, 45 minutes, 1 hour, 2 hours, 4 hours, 6 hours, 12 hours or 24 hours or more after) the administration of a second or third therapy or therapeutic agent to a subject in need of treatment as disclosed herein. The therapies (i.e. therapeutic agents, particularly two or more of a melatonin agent, a prolyl hydroxylase domain inhibitor (PHD inhibitor) are administered to a subject in a sequence and within a time interval such that the therapies can act together. In a particular embodiment, the therapies are administered to a subject in a sequence and within a time interval such that they provide an increased benefit than if they were administered otherwise.

“Effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating”, and “treatment” refer to any indicia of success in the treatment or amelioration of an injury, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. The treatment or amelioration of symptoms can be based on objective or subjective parameters; including the results of a physical examination, neuropsychiatric exams, and/or a psychiatric evaluation.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, the patient is human.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors and colors.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“A,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

The compositions of the present disclosure can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present disclosure can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present disclosure can be administered transdermally. The compositions of this disclosure can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

For preparing pharmaceutical compositions of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active components are mixed with the carrier having the necessary binding properties in suitable proportions and compacted a particular shape and size.

The powders, capsules and tablets preferably contain from about 5% to about 70% of the active compound, such as about 10% to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. Disintegrating or solubilizing agents may be added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the therapeutic agent(s) (e.g. erythropoietin and related mimetics, melatonin, PHD inhibitors) in water and adding optional suitable colorants, flavors, stabilizers, and thickening agents. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the therapeutic agent(s) (e.g. erythropoietin and related mimetics, melatonin, PHD inhibitors) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the present disclosure can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present disclosure can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

In another embodiment, the compositions of the present disclosure can be formulated for parenteral administration, such as intravenous (IV) administration or administration into a body cavity or lumen of an organ. The formulations for administration will commonly comprise a solution of the compositions of the present disclosure dissolved in a pharmaceutically acceptable carrier. Among the acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by various sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a sterile injectable solution or suspension in a nontoxic parenterally-acceptable diluent or solvent, such as a solution of 1,3-butanediol.

For methods and compositions for treating a subject suffering from or susceptible to prenatal opioid exposure and including for the treatment of brain injury associated with in utero and perinatal opioid exposure, suitable subject can include a woman who uses an opioid (e.g. a person who takes heroin, fentanyl or other opioid, or is a user or has used methadone, buprenorphine, naloxone or other medication-assisted therapy (MAT) for opiate addiction) and may or may not be pregnant. The woman may be capable of becoming pregnant or tested positive for being pregnant. In certain aspects, the subject is selected for treatment on the basis of being an opioid user (e.g. a person who takes heroin, fentanyl or other opioid or is a user of methadone, buprenorphine, naloxone or other medication-assisted therapy (MAT) for opioid addiction) and tested positive for being pregnant.

Additionally, for methods and compositions for treating a subject suffering from or susceptible to prenatal opioid exposure and including for the treatment of brain injury associated with in utero and perinatal opioid exposure, subjects may include a child (including an infant with the present therapy commencing within 0.25, 0.5, 1, 2, 3, 4, 5, 6, 7 days or more following birth) whose mother used an opioid (e.g. a person who is or has been a user of heroin, fentanyl or other opioid, or takes or has taken methadone, buprenorphine, naloxone or other agent for medication-assisted therapy (MAT) for opioid addiction) at any time during pregnancy prior to the child's birth, or within 1, 2, 3, 4, 5, 6, 7, 8, 9, 10, 11 or 12 months or more prior to becoming pregnant with the child.

In certain aspects, the subject will be a child (including an infant with the present therapy commencing within 0.25, 0.5, 1, 2, 3, 4, 5, 6, 7 days or more following birth) whose mother used medication-assisted therapy (MAT) for opioid addiction such as methadone, buprenorphine, naloxone, during pregnancy with the child.

In other certain aspects, the subject may be a woman who for example tests positive for being pregnant or is capable of becoming pregnant and who is or has been a user of heroin, fentanyl, or other opioid, or takes methadone, buprenorphine, naloxone or other agent for medication-assisted therapy (MAT) for opiate addiction at any time during pregnancy prior to the child's birth, or within 1, 2, 3, 4, 5, 6, 7, 8, 9, 10, 11 or 12 months or more prior to becoming pregnant with the child.

The subject also may be a child who has been identified as suffering from neonatal opioid withdrawal syndrome (NOWS).

The subject also may be a child who has been identified as suffering from neonatal abstinence syndrome (NAS).

The subject also may be a child identified as at risk for or potentially suffering from brain injury associated with in utero and perinatal opioid exposure.

The subject also may be a child identified as at risk or suffering from perinatal brain injuries, including cerebral palsy, perinatal traumatic brain injury and acquired forms of hydrocephalus including posthemorrhagic hydrocephalus, postinfectious hydrocephalus, and post-traumatic hydrocephalus.

In particular aspects, the subject also may be a child identified as at risk or suffering from cerebral palsy and related neurodevelopmental disorders.

The subject also may be a child including an infant identified as at risk or having been exposed an opioid, particularly having ingested an opioid.

The subject (for example, woman who tests positive for being pregnant or is capable of becoming pregnant, or a child including infant) may receive a wide range of dosage of a melatonin agent (sometimes referred to MLT herein). For instance, a melatonin agent may be suitably administered in amounts of 1, 2, 3, 4 to 5 mg/kg of patient bodyweight to 50, 60, 70, 80, 90 or 100 or more mg/kg of patient bodyweight.

The subject (for example, woman who tests positive for being pregnant or is capable of becoming pregnant, or a child including infant who has been exposed to opioids including prenatal exposure) also may receive a wide range of dosage of a melatonin agent (sometimes referred to MLT herein). For instance, a melatonin agent may be suitably administered in amounts of 1, 2, 3, 4 to 5 mg/kg of patient bodyweight to 50, 60, 70, 80, 90 or 100 or more mg/kg of a subject's bodyweight.

The subject (for example, woman who tests positive for being pregnant or is capable of becoming pregnant, or a child including infant who has been exposed to opioids including prenatal exposure) also may receive a wide range of dosage of an erythropoietin compound. For instance, current reported dosages usages of an erythropoietin compound for other treatment protocols may be employed. In one protocol, rhEPO may be administered at 400 U/kg/dose to 1500 U/kg/dose, such as 1000 U/kg/dose.

The subject (woman who tests positive for being pregnant or capable of becoming pregnant, or a child including infant who has been exposed to opioids including prenatal exposure) also may receive a wide range of dosage of one or more prolyl hydroxylase domain inhibitor (PHD inhibitor) compounds. For instance, current reported dosages usages of a prolyl hydroxylase domain inhibitor (PHD inhibitor) compound such as Roxadustat for other treatment protocols may be employed for the present therapies, including about 10, 20, 30, 40, 50, 60, 70, 80, 90, 100 mg or more per day per subject.

In this aspect, the subject (woman who tests positive for being pregnant or capable of becoming pregnant, or a child including infant) suitably may be administered the one or more therapeutic agents on a schedule as desired including once a day, or multiple daily administrations and for a single day or more extended schedules such as 2, 3, 4, 5, 6, or 7 days, or 1, 2, 3, 4, 5, 6, 7, 8, 9, 10, 11, 12 or more weeks.

The above dosages and schedule of administration also may be used for treating a subject suffering from or susceptible traumatic brain injury, intracranial hemorrhage, acquired hydrocephalus, demyelinating diseases including multiple sclerosis, neurological symptoms from systemic lupus erythematosus, and neurodegenerative diseases. Typical subjects may be human subjects of any age, including children (e.g. 16 years old or younger) as well as adults (e.g. ages 16, 18 or 21 years or more).

The above dosages and schedule of administration also may be used to treat adult brain insults including hydrocephalus, brain injury (including TBI) and other neurodegenerative insults. Typical subjects may be human subjects of any age, including children (e.g. 16 years old or younger) as well as adults (e.g. ages 16, 18 or 21 years or more).

For treating subject having a COVID-19 infection, exhibiting symptoms of a COVID-19 infection, having suspected exposure to COVID-19 or having suspected post-acute sequelae of COVID-19 (PASC), a melatonin agent and one or more propyl hydroxylase domain inhibitor (PHD inhibitor) compounds may be suitably administered in dosages and amounts as set forth above.

As discussed, the present methods and compositions can be used to treat a subject identified as suffering from long COVID or post-acute sequelae of COVID-19 (PASC), e.g. where the subject exhibits the persistence of COVID-19 symptoms for four weeks or longer after the initial onset or diagnosis of a COVID-19 infection, or chronic or post-COVID-19 syndrome or coronavirus infection, which includes symptoms and abnormalities persisting or present beyond 12 weeks of the onset of acute COVID-19 or coronavirus infection and not attributable to alternative diagnoses. Symptoms of long COVID or post-acute sequelae of COVID-19 (PASC) may include: fatigue; shortness of breath or difficulty breathing; cough; joint pain; chest pain; memory, concentration or sleep problems; muscle pain or headache; fast or pounding heartbeat; loss of smell or taste; depression or anxiety; fever; dizziness while standing; and/or worsened symptoms after physical or mental activities.

Exemplary suitable dosages and administration protocols are also set forth in the examples which follow.

In a further aspects, the compostions and methods of the invention can be used for non-surgical medical treatment of infants (including preterm and very preterm infants) with intraventricular hemorrhage (include severe intraventricular hemorrhage (sIVH)). Administration of the present compositions and methods to such subjects can result in the subjects spontaneously recovering their own CSF management and dynamics, and/or to decrease the need for surgery for posthemorrhagic hydrocephalus (PPH).

The following examples are illustrative.

### Example 1: In Vivo Studies

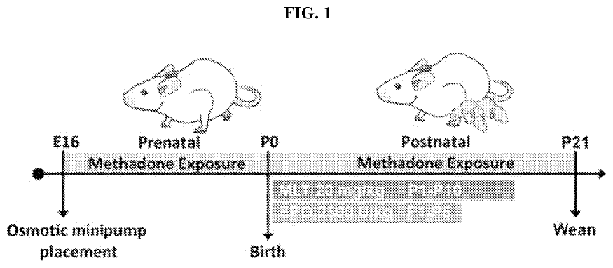

We developed a preclinical model of perinatal methadone exposure. A unique challenge to neurodevelopmental research is accurately recapitulating the intact maternal-placental-fetal unit, in which the compartment-specific responses to specific insults such as opioids can be accurately studied. On embryonic day 16 (E16) prior to complete oligodendrocyte, microglial and astrocyte maturation,¹⁵⁰ osmotic mini pumps are implanted in pregnant dams for 28 days of continuous methadone or saline infusion. On E22, rat pups are born and remain with their dams, receiving methadone through the maternal milk supply. The first two-weeks in rat postnatal life is equivalent to the human third trimester (P10 is roughly human term equivalent).¹⁵⁰ Thus, opioid exposure is continued through the perinatal period via milk. Similar to what is observed in human neonates,¹⁵¹⁻¹⁵³ rat pups exposed to methadone in utero and continuing through the perinatal period have significantly reduced body weight.²⁷ Here, we will extend this model to include neonatal EPO+MLT treatment (FIG. 1). A cohort of methadone-exposed pups will received EPO from postnatal day 1 (P1)-P5 at 2500 U/kg/dose and MLT from P1-P10 at 20 mg/kg consistent with similar studies of neurorepair in FIG. 1.

### Methadone Induces Functional Brain Injury.

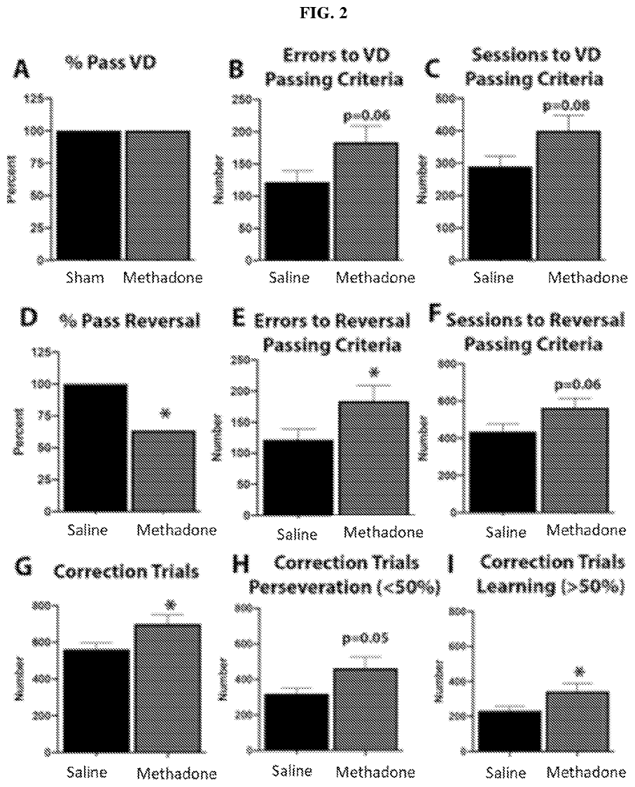

Children born to women who have been prescribed methadone are at risk of neurodevelopmental impairment.^{45,46} Thus, to define functional consequences of methadone exposure on the CNS, we used a translatable touchscreen operant platform to assess cognition and executive function in adult rats following in utero methadone exposure (FIG. 2). Rats exposed to perinatal methadone, and saline control rats, were able complete a task of visual discrimination (a task analogous to intra/extra dimensional set shifting on human touchscreen Cambridge Neuropsychological Test Automated Battery (CANTAB)).⁷⁶ However, rats with perinatal methadone exposure were impaired in both early and late reversal teaming, consistent with widespread learning and executive control dysfunction, including lack of cognitive flexibility (FIG. 2). Notably, adult rats exposed to methadone in the perinatal period performed markedly slower in the acquisition of pairwise visual discrimination in adulthood compared to controls. Moreover, when the reinforced contingencies of the learned association were reversed, methadone rats were significantly impaired compared to controls. Analysis of reversal performance by stage revealed that methadone rats were impaired both on the early, perseverative phase of reversal, as well as the later learning stage. Numerous previous studies have shown that discrimination, as well as the learning stage of reversal, is mediated primarily by the dorsal striatum, while efficient early reversal requires intact cortical functioning.¹⁵⁴⁻¹⁵⁷ Our results suggest that methadone significantly impairs both the efficient learning of a new association during discrimination, as well as cortically-mediated early phases of reversal. Importantly, these impairments were not due to sensorimotor-related performance or nonspecific lack of motivation, as evidenced by normal scores on response reaction times and reward retrieval latencies.²⁷ Collectively, these data show that methadone exposure in utero leads to long term impairments in both associative learning and executive control. Perhaps more compelling, however, is that these data support clinical literature, and are in direct alignment with a recent meta-analysis demonstrating prenatal opioid exposure is negatively associated with neurocognitive and physical development,¹⁵⁸ along with numerous other smaller studies.¹⁵⁹⁻¹⁶² Specifically, prenatal opioid exposure is associated with lower cognitive scores, with the largest differences observed between ages 6 months and 6 years. Indeed, data show that neurodevelopment does not improve after preschool and worsens by school age.¹⁵⁸ These data are also similar to that of humans with early-life opioid exposure and survivors of perinatal brain injury who fail to pass touchscreen assessments of dimensional shift as the tasks become more complex.^{46,163-165} Together, these data confirm that gestational opioid exposure during crucial in utero and perinatal periods of neurodevelopment have lasting and permanent changes in brain structure and function. Now that we have established these deficits in cognitive control and executive function, our next step will be to assess whether a postnatal administration of EPO+MLT will mitigate these cognitive deficits similar to what we have observed in our rat model of cerebral palsy secondary to chorioamnionitis.²⁶ Importantly, touchscreen is a highly sensitive behavioral assessment that detects differences in unique forms of perinatal brain injury defined by specific circuit abnormalities.

### EPO+MLT Treatment Mitigates Structural Brain Injury Following Perinatal Opioid Exposure.

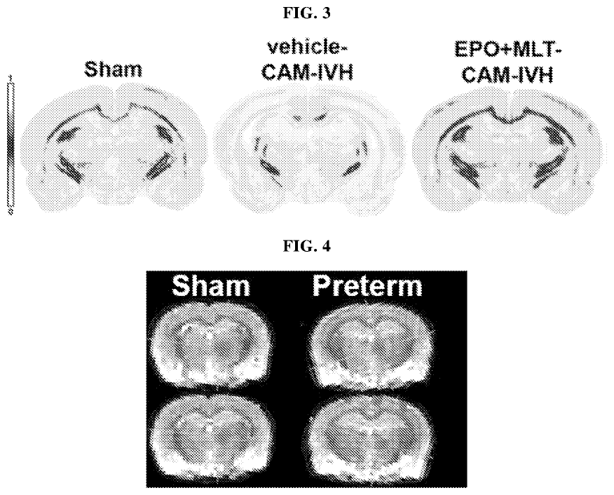

In pilot experiments conducted specifically for this proposal, we performed DTI to assess white matter injury in adult rats exposed to methadone in the perinatal period. In adulthood, methadone exposed rat offspring have abnormal fractional anisotropy (FA, FIG. 3). Significantly, we found loss of FA, disrupted microstructural integrity, and impaired structural coherence was prevented with neonatal EPO+MLT treatment (FIG. 3). These data fully align with clinical literature demonstrating a similar pattern of diffusion MRI abnormalities in children exposed to methadone in utero and in adults with chronic methadone exposure.^{45,166,167} Using diffusion tensor imaging in other models of PBI we have correlated DTI abnormalities with phenotype and treatment.^{28,80} Further, we will focus our in vivo and ex vivo DTI analyses on microstructural abnormalities in brain regions implicated in cognition and executive function168-170, including the frontal cortex, striatum, thalamic reticular nucleus, thalamus, and the thalamocortical tracts. At the Small Animal Imaging Facility at the Kirby Center at the Kennedy Krieger Institute we are also performing in vivo functional connectivity (FIG. 4). We created a pipeline to test changes in fcMRI in methadone exposed animals with and without treatment similar to our studies in other forms of perinatal brain injury.

These data from our lab and others confirm that opioid exposure during crucial in utero and perinatal periods of neurodevelopment induce lasting and permanent changes in brain structure and function in rats.^{27,44} Perinatal methadone exposure causes decreased expression of MBP and altered pNF:NF expression, synonymous with impaired myelination and axonal injury, similar to that which has been reported in animal models of placental insufficiency and chorioamnionitis.^{28,81} Notably, buprenorphine and methadone disrupt complex sequences of molecular events essential to connectivity in the developing brain, including oligodendrocyte maturation and timing of myelination.^{171,172} FA is a marker of tract microstructure that reflects fiber density, axonal diameter, wrapping by pre-myelinating oligodendrocytes and myelination⁴⁵ Therefore, these data suggest that rats exposed to perinatal methadone have more immature, less coherently organized fiber tracts compared to controls, consistent with microstructural brain injury. Our data are similar to those in human neonates exposed to methadone in utero,⁴⁵ and are consistent with the observation that FA is reduced through the white matter of neonates born to women who were prescribed methadone.^{45,48} Significantly, pilot data supports our hypothesis that EPO+MLT treatment reduces microstructural brain injury in adulthood after perinatal opioid exposure.

### EPO+MLT Reduces Methadone-Induced Increases in Inflammatory Cytokines in Peripheral Circulation.

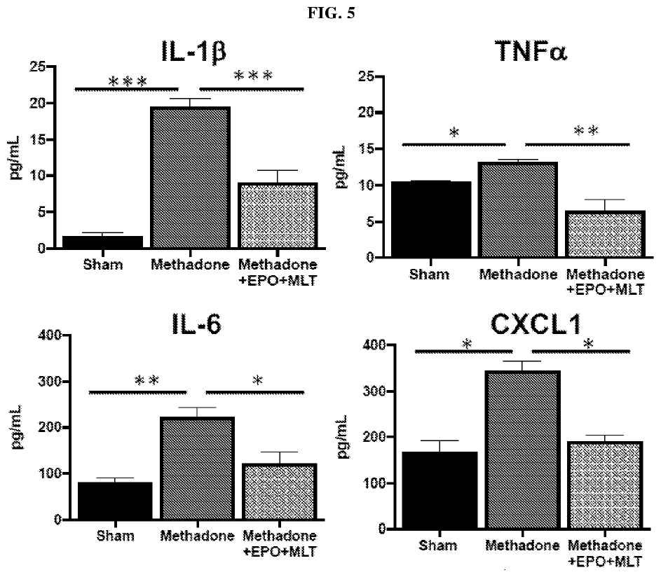

A systemic inflammatory response syndrome (SIRS) is defined by a robust and diverse systemic inflammatory protein network profile, hallmarked by increased concentration of cytokines in the circulation, including upregulation of IL-6, TNFa and IL-1b.¹⁷³⁻¹⁷⁹ We found that methadone induces a SIRS that, like in humans, lasts well in to the postnatal period.2^{7,180-185} It is well established that risk of brain injury is increased in infants who have persistent and/or recurrent elevations of pro-inflammatory proteins.¹⁸⁶ Specifically, the increasing breadth of early neonatal inflammation, indexed by the number of protein elevations or the number of functional protein classes elevated, is associated with increased structural and functional brain injury.^{180,182,186-189} Notably, the administration of EPO+MLT mitigated the pronounced inflammatory profile in our rats, and reduced systemic inflammation defined by increases in CXCL1, IL-6, TNFa and IL-1b (FIG. 5). This decrease supports the hypothesis that perinatal methadone-induced inflammation can be modulated by exogenous EPO+MLT treatment, and that these cytokines may prove useful as serum biomarkers to predict the propensity to develop perinatal opioid induced brain injury or responsiveness to treatment.

### EPO+MLT Attenuates Aberrant Opioid-Induced Lymphocyte Reactivity.

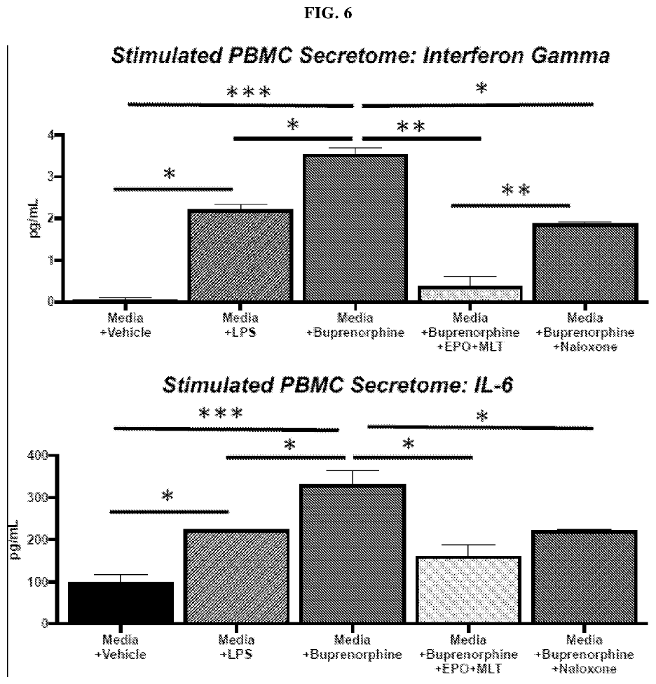

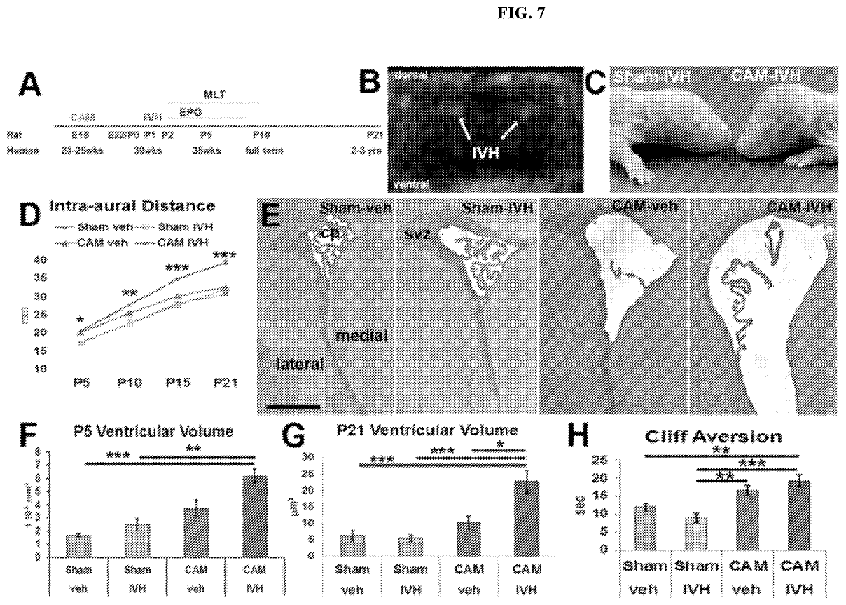

Like the CNS, the immune system develops and matures over the course of gestation and perinatal period.¹⁷⁴ Dysregulated chemokines and cytokines, and changes in immune cells themselves, culminate in impaired immune function that can last decades.^{149,190} Similar to neural cells, leukocytes are uniquely responsive to their environment. Thus, understanding the homeostatic regulation of central and peripheral inflammatory cells in infants following opioid exposure and the long-term consequences of their dysregulation is essential.^{174,191} As we have previously published the effects of methadone on PBMCs and demonstrated a significant priming effect of methadone and an SPIHR,²⁷ we examined the effect of buprenorphine on these circulating cells here to confirm a causal neuroinflammatory effect of opioids in general. Pilot data generated for this proposal indicate that PBMCs secrete pro-inflammatory mediators both in response to LPS, a known inflammatory stimulus, and buprenorphine (FIG. 6). Interestingly, buprenorphine induced a more robust secretion of inflammatory mediators compared to LPS. Importantly, co-culture with EPO+MLT and buprenorphine reduced the secretion of interferon gamma and IL-6, cytokines essential to immune cell trafficking and inflammatory signal transduction.

Using our validated and published model of perinatal opioid exposure, we determine if a combination therapy of EPO+MLT commencing in the neonatal period will attenuate SIRS and SPIHR, limit white matter and gray matter brain injury as seen on MRI, improve microstructural and network coherence, and mitigate deficits of executive function and cognitive control. Our preliminary data support this central hypothesis and provide the proof of concept data supporting that EPO+MLT are clinically relevant therapeutic targets in need of further validation, with a focus on translational, functional outcomes.

### Specific Aim 1

Rationale. Test the hypothesis that neonatal combined EPO+MLT therapy attenuates deficits of cognitive control and executive function in adult animals following perinatal methadone exposure. Opioid induced inflammation during critical windows of intrauterine and extrauterine life is a pathologic force that can drive alterations of postnatal innate and adaptive immunity and brain development.¹⁹⁶ Indeed, in utero priming or activation of the fetal immune system at critical developmental time can lead to chronic inflammatory disorders, increased vulnerability to infection after birth¹⁹⁶ structural brain injury and impairment in cognition and executive function. We hypothesize that gestational opioid exposure is defined by excess inflammatory signaling throughout crucial periods of CNS development that is toxic to developing neural cells leading to cognitive impairment in adulthood. We predict that EPO+MLT will attenuate early and late stage reversal learning deficits, and improve learning and cognitive control in adult rats with perinatal methadone exposure.

Methods. Prenatal and Perinatal Methadone Exposure. On embryonic day 16 (E16), a developmental time point equivalent to the late second trimester, prior to oligodendrocyte, microglial and astrocyte maturation,^{72,150} osmotic mini pumps will be implanted subcutaneously in pregnant Sprague Dawley rats for continuous methadone infusion (12 mg/kg, 0.25 μL/hour flow rate for 28 days) Pups are born at E22 and remain with their dams, receiving methadone through milk until weaning (P21,Model Summary). We have previously published maternal and rat pup urine and serum levels.²⁷ Our pilot data has shown that litter sizes remain constant with an average of 12 pups/litter/group. Pups are weighed and monitored daily for feeding. Litter sizes will be monitored and the sex of every pup recorded.

EPO+MLT Treatment. A cohort of methadone-exposed pups will received EPO from postnatal day 1 (P1)-P5 at 2500 U/kg/dose and MLT from P1-P10 at 20 mg/kg consistent with similar studies of neurorepair in neonatal animals.^{6,26} This dosing regimen will capitalize on EPO+MLT's multiple mechanisms of action including reduction of oxidative and inflammatory stress, promotion of neural cell development, and regeneration of lost neural cells. Extended dosing regimens with repeated high doses of EPO have typically shown to be the most beneficial for repair.79,197

Touchscreen Testing of Cognition and Executive Function. We will test visual discrimination (VD) and reversal learning to quantify cognitive flexibility, per our prior work.^{5,6,26} Post-weaning, rats will begin mild food restriction at P28, and touchscreen training at P35 consistent with our published methods.^{5,6,26} After rats complete training, they undergo VD testing, followed by assessment of reversal learning.^{5,6,73-75,77,154,198} Passing criteria is set a priori as 80% correct for two consecutive days. ^{5,6,26} Our pilot and published data²⁷ demonstrate the feasibility of this approach in perinatal methadone exposure.

Statistical Analysis for Aim 1. In all of our work, all outcome measures are independent and orthogonal. Normal distribution will be verified with the Shapiro-Wilk test, with Levene's test to confirm homogeneity of variances. Two group comparisons will be performed by a Student's t-test for normally distributed data, whereas Mann-Whitney U tests will be used in the absence of normal distribution. Multiple comparisons in non-normally distributed data will be performed using Kruskal Wallis followed by Dunn's post-hoc analysis. Two-Way ANOVA with Bonferroni correction will be used to assess the effects of treatment with EPO+MLT and methadone exposure. With respect to animal number, as we have previously published, the ideal group size per condition is 10 (10 males and 10 females) assuming a power of 0.8 and an a of 0.05 for multiple group comparisons.^{2,3,28,81,92,173,192-194} In determining the number of samples per group, we used G* power. The number of samples needed for 80% power in a two tailed independent t test was determined using the data from which there is the most variable data set with the lowest effect size. The calculated Cohen's d for this data is 1.375 (a mean difference of 0.11 and an average SD of 0.08).

Potential Problems and Alternative Approaches. Opioid exposure is common at all gestational ages. However, this model represents methadone exposure commencing prior to major events of myelination, neural circuit formation and connectivity, continuing through the perinatal period. While this does not recapitulate every infant's experience of gestational opioid exposure, rodents experience their third trimester equivalent ex utero. Despite this key species difference, data generated will be useful for all fetuses, infants and children exposed to opioids with inflammatory pathophysiology. Specifically, it lays the foundation for future studies of methadone commencing prior to pregnancy, and other opioids including buprenorphine and oxycodone. Buprenorphine is a semisynthetic derivative, which selectively binds to the μ-opioid receptor as a partial agonist, and to the K-opioid receptor as an antagonist. While it will be essential to investigate buprenorphine and prescription opioids in future studies, we elected to start with methadone because of its full agonist properties and use in our patient populations. Inflammation is ubiquitous with exposure to all opioids and the findings of these studies will be widely applicable, and have implications for exposure to any prescription or non-prescription opioids impacting fetuses, neonates and young children.

### Specific Aim 2

Rationale. Test the hypothesis that combined EPO+MLT therapy mitigates structural and functional brain injury in adulthood following perinatal methadone exposure. We predict that animals exposed to opioids commencing prenatally will have fragmented neural networks and disrupted anatomical connectivity. We expect there to be reduced functional connectivity in regions essential to cognition, including reduced association between cortico-thalamic, cortico-cortico, and cortico-striatal pathways. We propose that EPO+MLT will mitigate white matter and gray matter injury, and improve microstructural coherence and diffusion on MRI concomitant with increased neural network refinement, regional association and sophistication.

Methods. MRI. We will assess the trajectory of brain development, injury, and putative recovery with EPO+MLT treatment through a critical neurodevelopmental window using MRI at P21, P60 and at the conclusion of touchscreen assessment in aim 1. These time points will cover key white matter and connectivity milestones in major white matter tracts, the prefrontal cortex, hippocampus, mediodorsal and reticular thalamic nuclei (brain regions essential to cognition).^{2,4,72,192,199-202} Ex vivo DTI analysis of anatomical connectivity altered by methadone will be assayed consistent with the clinical literature,⁴⁵ and our previous publications.^{4,27,193,194,202} We will also perform in vivo DTI analysis at the conclusion of cognitive assessment in Aim 1. All diffusion scalars and parameters, including fractional anisotropy (FA), mean diffusivity, and axial and radial diffusivity will be assessed consistent with our published methods.^{4-6,28,29,193} Ex vivo imaging affords the unique ability of being able to perform pathological analyses after imaging in the same brain, and provides strong correlation between standard histopathology and highly translatable MRI metrics. These studies will be performed if it is validated that EPO+MLT reverses the behavioral phenotype described in Aim 1.

In vivo functional connectivity. Functional Connectivity will be assessed per pilot data and as described above at P21, P60 and at the conclusion of touchscreen assessment in aim 1. Rodents are sedated with dexmedetomidine to minimize confounding effects from isoflurane. Images are acquired in the Kirby Animal Imaging Facility. Imaging parameters are T2w: TR 3724, TE 30 coronal acquisition; 0.4 mm isotropic; matrix size 73×73, 39 slices; acquisition time 33 sec); fat suppression on; and fcMRI: TR1000, TE 14.6, coronal acquisition; 0.4 mm isotropic (same space as T2w), 451 vol (15 min acquisition); fat suppression on. Data will be post-processed using AFNI (despiking, application of a 0.01-0.05 Hz bandpass filter, and spatial blurring with a Gaussian kernel with full width at half maximum of 1.2 mm). Connectivity between brain regions will be visualized using AFNI InstaCorr. Similar to pilot data, correlations among voxels will be color coded and displayed at a level of p<0.05 uncorrected.

Statistical Analysis for Aim 2. Statistical analyses will be completed per Aim 1.

### Potential Problems and Alternative Approaches.

We are aware that clinical trials of EPO for the treatment of severe brain injury in adults have failed. It is likely that dosing regimens were too low or of insufficient dosing intensity to produce neurological improvement. In addition to incomplete dosing regimens, single endpoints with differential timing of primary outcome measures, and lack of sensitive outcome measures despite severe injury at initiation of treatment, have contributed to trial failures.²⁰³ Three meta-analyses of EPO for the treatment of severe adult traumatic brain injury have recently been published.²⁰⁴⁻²⁰⁶ Importantly, no adverse events were evident compared to placebo treatment. ^{204,205,207} Both the Australian EPO-TBI trial (NCT00987454) and the US EPO trial (NCT003131716) tried to balance the perceived risks of complications in adults, including increased hematocrit and thromboses, with the need to achieve high-dose, extended therapy, which may have led to under dosing and lack of efficacy. ²⁰⁸ Together, these findings emphasize the need for careful clinical trial design and consideration of the unique pediatric patient population. Notably, no issues with hematocrit, pathological clotting, or thromboses are reported in any pediatric trial of EPO treatment and no adverse events have emerged with high-dose, extended EPO regimens in neonatal trials.139,144-147

### Specific Aim 3

Rationale. Test the hypothesis that combined EPO+MLT therapy diminishes a sustained systemic pro-inflammatory microenvironment propagated by perinatal methadone exposure. Like the CNS, the immune system develops and matures over the course of gestation and throughout childhood.¹⁷⁴ Dysregulation in neural-immune communication and programming has been reported in several disorders, including chronic pain, fetal alcohol syndrome, stroke, schizophrenia, Alzheimer's disease and autism spectrum disorders. ²⁰⁹-2¹⁶ Notably, persistent peripheral immune activation, immune cell priming, and immune cell hyperactivity is associated with tertiary brain injury in neonates, fragmented neural networks and functional perinatal injury.^{149,180,217-219} We hypothesize that EPO+MLT will attenuate the systemic pro-inflammatory cytokines response and sustained peripheral immune hyper-reactivity (SPIHR) opioids induce throughout postnatal development.

Methods. MECI. Serum will be collected along an extended time course from P2 to P15, P21 and P60 to cover major milestones of late fetal development, immune system maturation, and bi-directional immune-brain cross talk during rapid periods of neural cell maturation and network formation. To measure cytokine and chemokines in serum from saline, methadone, and methadone+EPO+MLT treated rats, a clinical multiplex electrochemiluminescent immunoassay platform will be used consistent with our prior studies and numerous similar human studies.^{4,173,182,183,186-188,220} Concentrations of major inflammatory biomarkers and immune trafficking proteins, including CXCL1, TNFa, IL-1b, IL-6, and MCP-1, and their receptors (CXCR2, TNFR1, TNFR2, IL-1R, and CCR2) will be assessed.

Peripheral Blood Mononuclear Cell (PBMC) Isolation. Fetal white blood cell counts change with gestational age, with lymphocytes being the most prevalent leukocyte through 37 weeks' gestation.^{174,221} While lymphocytes increase linearly with gestational age, neutrophils increase exponentially after 31 weeks' gestation and become the predominant lymphocyte at term.¹⁷⁴ Thus, PBMCs from saline, methadone, and methadone+EPO+MLT treated rats will be isolated on P7 (approximately 37 weeks' gestation), P21 (older infant), P60 (mature adult), and at the conclusion of touchscreen testing in Aim 1 (˜P90). Ficoll gradient separation will be used consistent with our pilot data and published methods.88,149

PBMC Treatment with Lipopolysaccharide (LPS). PBMCs culture will be performed consistent with pilot data and our published work.⁸⁸ At P7, P21, P60, and P90 PBMCs from saline, methadone, and methadone+EPO+MLT rats will be plated at a density of 2×10⁶ cells per dish (1×10⁶ cells/mL, 2 mL each) and challenged without or with LPS (100 ng/mL) for 3 h or 24 h to quantify SPIHR. The supernatants will then be collected consistent with prior reports.149,222

PBMC Treatment with Methadone or Buprenorphine. To test the direct effect of methadone on the PBMC secrotome and reactivity, we will also stimulate PBMCs in culture with methadone and buprenorphine directly consistent with pilot data. As described above and similar to LPS, methadone or buprenorphine (10 μM) will be applied to PBMCs in culture. For blocking experiments, naloxone (10 μM), EPO (10 U/mL) and/or MLT (20 μM) will be added 2 hours before methadone, buprenorphine or LPS. We predict that PBMCs treated with EPO+MLT will be less reactive when challenged with methadone or buprenorphine and secrete fewer cytokines in culture such as TNFa, IL-1B, and IL-6.

PBMC Secretome Analysis. To evaluate the PBMC secretome, a secreted cytokine and chemokine profile analysis will be performed on supernatants from cultured PBMCs or serum using a V-plex rat pro-inflammatory panel as described above. This methodology is consistent with pilot data and published methods, and numerous prior preclinical and clinical studies.^{4,28,88,173,180,182,186,188} We expect EPO+MLT treated rats exposed to methadone will have a blunted inflammatory cytokine/chemokine signature in serum and PBMC secretome throughout our time course.

Statistical Analysis for Aim 3 will be completed as per Aim 1.

### Potential Problems and Alternative Approaches.

Immune activation increases expression of inflammatory mediators such as IL-1$R, TNFa, IL-6, MCP-1 and CXCL1. However, not all inflammation is detrimental and many cytokines and chemokines have essential roles in immune and CNS development.29,30,88 223,22⁴Immune response is context specific, varies with developmental stage, occurs along a graded continuum and is tightly regulated. To this end, these mediators act to eliminate pathology and enhance repair, but may also directly induce damage.²²¹ Similarly, immune cells themselves can also be directly involved in injury.²²⁶⁻²²⁸ In acknowledgement that the role of each cytokine and immune cell is multifactorial, we will evaluate each in the context of normal development, methadone exposure, and EPO+MLT treatment.

### REFERENCES FOR EXAMPLE 1

1. Honein M A, Boyle C, Redfield R R. Public Health Surveillance of Prenatal Opioid Exposure in Mothers and Infants. Pediatrics 2019; 143.
2. Jantzie L L, Corbett C J, Firl D J, Robinson S. Postnatal Erythropoietin Mitigates Impaired Cerebral Cortical Development Following Subplate Loss from Prenatal Hypoxia-Ischemia. Cereb Cortex 2015; 25:2683-95.
3. Jantzie L L, Getsy P M, Firl D J, Wilson C G, Miller R H, Robinson S. Erythropoietin attenuates loss of potassium chloride co-transporters following prenatal brain injury. Molecular and cellular neurosciences 2014; 61:152-62.
4. Robinson S, Winer J L, Berkner J, Chan L A, Denson J L, Maxwell J R, Yang Y, Sillerud L O, Tasker R C, Meehan W P, 3rd, Mannix R, Jantzie L L. Imaging and serum biomarkers reflecting the functional efficacy of extended erythropoietin treatment in rats following infantile traumatic brain injury. Journal of neurosurgery Pediatrics 2016; 17:739-55.
5. Robinson S, Winer J L, Chan L A S, Oppong A Y, Yellowhair T R, Maxwell J R, Andrews N, Yang Y, Sillerud L O, Meehan W P, 3rd, Mannix R, Brigman J L, Jantzie L L. Extended Erythropoietin Treatment Prevents Chronic Executive Functional and Microstructural Deficits Following Early Severe Traumatic Brain Injury in Rats. Frontiers in neurology 2018; 9:451.
6. Robinson S, Conteh F S, Oppong A Y, Yellowhair T R, Newville J C, Demerdash N E, Shrock C L, Maxwell J R, Jett S, Northington F J, Jantzie L L. Extended Combined Neonatal Treatment With Erythropoietin Plus Melatonin Prevents Posthemorrhagic Hydrocephalus of Prematurity in Rats. Front Cell Neurosci 2018; 12:322.
7. Lowe J R, Rieger R E, Moss N C, Yeo R A, Winter S, Patel S, Phillips J, Campbell R, Baker S, Gonzales S, Ohls R K. Impact of Erythropoiesis-Stimulating Agents on Behavioral Measures in Children Born Preterm. J Pediatr 2017; 184:75-80 e1.
8. Messier A M, Ohls R K. Neuroprotective effects of erythropoiesis-stimulating agents in term and pretermneonates. Curr Opin Pediatr 2014; 26:139-45.
9. Ohls R K, Christensen R D, Widness J A, Juul S E. Erythropoiesis Stimulating Agents Demonstrate Safety and Show Promise as Neuroprotective Agents in Neonates. J Pediatr
2015; 167:10-2.
10. Phillips J, Yeo R A, Caprihan A, Cannon D C, Patel S, Winter S, Steffen M, Campbell R, Wiedmeier S, Baker S, Gonzales S, Lowe J, Ohls R K. Neuroimaging in former preterm children who received erythropoiesis stimulating agents. Pediatric research 2017; 82:685-90.
11. Juul S E, Comstock B A, Heagerty P J, Mayock D E, Goodman A M, Hauge S, Gonzalez F, Wu Y W. High-Dose Erythropoietin for Asphyxia and Encephalopathy (HEAL): A Randomized Controlled Trial—Background, Aims, and Study Protocol. Neonatology 2018; 113:331-8.
12. Mulkey S B, Ramakrishnaiah R H, McKinstry R C, Chang T, Mathur A M, Mayock D E, Van Meurs K P, Schaefer G B, Luo C, Bai S, Juul S E, Wu Y W. Erythropoietin and Brain Magnetic Resonance Imaging Findings in Hypoxic-Ischemic Encephalopathy: Volume of Acute Brain Injury and 1-Year Neurodevelopmental Outcome. J Pediatr 2017; 186:196-9.
13. Wu Y W, Bauer L A, Ballard R A, Ferriero D M, Glidden D V, Mayock D E, Chang T, Durand D J, Song D, Bonifacio S L, Gonzalez F F, Glass H C, Juul S E. Erythropoietin for neuroprotection in neonatal encephalopathy: safety and pharmacokinetics. Pediatrics 2012; 130:683-91.
14. Wu Y W, Gonzalez F F. Erythropoietin: a novel therapy for hypoxic-ischaemic encephalopathy? Developmental medicine and child neurology 2015; 57 Suppl 3:34-9.
15. Wu Y W, Mathur A M, Chang T, McKinstry R C, Mulkey S B, Mayock D E, Van Meurs K P, Rogers E E, Gonzalez F F, Comstock B A, Juul S E, Msall M E, Bonifacio S L, Glass H C, Massaro A N, Dong L, Tan K W, Heagerty P J, Ballard R A. High-Dose Erythropoietin and Hypothermia for Hypoxic-Ischemic Encephalopathy: A Phase II Trial. Pediatrics 2016; 137.
16. Juul S E, Comstock B A, Wadhawan R, Mayock D E, Courtney S E, Robinson T, Ahmad K A, Bendel-Stenzel E, Baserga M, LaGamma E F, Downey L C, Rao R, Fahim N, Lampland A, Frantz Iii I D, Khan J Y, Weiss M, Gilmore M M, Ohls R K, Srinivasan N, Perez J E, McKay V, Vu P T, Lowe J, Kuban K, O'Shea T M, Hartman A L, Heagerty P J, Consortium P T. A Randomized Trial of Erythropoietin for Neuroprotection in Preterm Infants. The New England journal of medicine 2020; 382:233-43.
17. Juul S E, Pet G C. Erythropoietin and Neonatal Neuroprotection. Clinics in perinatology 2015; 42:469-81.
18. Carloni S, Albertini M C, Galluzzi L, Buonocore G, Proietti F, Balduini W. Melatonin reduces endoplasmic reticulum stress and preserves sirtuin 1 expression in neuronal cells of newborn rats after hypoxia-ischemia. J Pineal Res 2014; 57:192-9.
19. Biran V, Decobert F, Bednarek N, Boizeau P, Benoist J F, Claustrat B, Barre J, Colella M, Frerot A, Garnotel R, Graesslin O, Haddad B, Launay J M, Schmitz T, Schroedt J, Virlouvet A L, Guilmin-Crepon S, Yacoubi A, Jacqz-Aigrain E, Gressens P, Alberti C, Baud O. Melatonin Levels in Preterm and Term Infants and Their Mothers. Int J Mol Sci 2019; 20.
20. Biran V, Phan Duy A, Decobert F, Bednarek N, Alberti C, Baud O. Is melatonin ready to be used in preterm infants as a neuroprotectant? Developmental medicine and child neurology 2014; 56:717-23.
21. Carloni S, Proietti F, Rocchi M, Longini M, Marseglia L, D'Angelo G, Balduini W, Gitto E, Buonocore G. Melatonin Pharmacokinetics Following Oral Administration in Preterm Neonates. Molecules 2017; 22.
22. Dominguez Rubio A P, Sordelli M S, Salazar A I, Aisemberg J, Bariani M V, Cella M, Rosenstein R E, Franchi A M. Melatonin prevents experimental preterm labor and increases offspring survival. J Pineal Res 2014; 56:154-62.
23. Lee J Y, Song H, Dash O, Park M, Shin N E, McLane M W, Lei J, Hwang J Y, Burd I. Administration of melatonin for prevention of preterm birth and fetal brain injury associated with premature birth in a mouse model. American journal of reproductive immunology 2019; 82:e13151.
24. Paprocka J, Kijonka M, Rzepka B, Sokol M. Melatonin in Hypoxic-Ischemic Brain Injury in Term and Preterm Babies. Int J Endocrinol 2019; 2019:9626715.
25. Yan C, Zhang B. Clinical significance of detecting serum melatonin and SBDPs in brain injury in preterm infants. Pediatr Neonatol 2019; 60:435-40.
26. Jantzie L L, Oppong A Y, Conteh F S, Yellowhair T R, Kim J, Fink G, Wolin A R, Northington F J, Robinson S. Repetitive Neonatal Erythropoietin and Melatonin Combinatorial Treatment Provides Sustained Repair of Functional Deficits in a Rat Model of Cerebral Palsy. Frontiers in neurology 2018; 9:233.
27. Jantzie L L, Maxwell J R, Newville J C, Yellowhair T R, Kitase Y, Madurai N, Ramachandra S, Bakhireva L N, Northington F J, Gerner G, Tekes A, Milio L A, Brigman J L, Robinson S, Allan A. Prenatal opioid exposure: The next neonatal neuroinflammatory disease. Brain, behavior, and immunity 2019.
28. Robinson S, Corbett C J, Winer J L, Chan L A S, Maxwell J R, Anstine C V, Yellowhair T R, Andrews N A, Yang Y, Sillerud L O, Jantzie L L. Neonatal erythropoietin mitigates impaired gait, social interaction and diffusion tensor imaging abnormalities in a rat model of prenatal brain injury. Experimental neurology 2017; 302:1-13.
29. Yellowhair T R, Noor S, Maxwell J R, Anstine C V, Oppong A Y, Robinson S, Milligan E D, Jantzie L L. Preclinical chorioamnionitis dysregulates CXCL1/CXCR2 signaling throughout the placental-fetal-brain axis. Experimental neurology 2018; 301:110-9.
30. Yellowhair T R, Newville J C, Noor S, Maxwell J R, Milligan E D, Robinson S, Jantzie L L. CXCR2 Blockade Mitigates Neural Cell Injury Following Preclinical Chorioamnionitis. Front Physiol 2019; 10:324.
31. Committee On Obstetric Practice and the American Society of Addiction Medicine. Opioid Use and Opioid Use Disorder in Pregnancy The American College of Obstetricians and Gynecologists 2017; ACOG Committee Opinion:August 2017.
32. Caritis S N, Panigrahy A. Opioids affect the fetal brain: refraining the detoxification debate. American journal of obstetrics and gynecology 2019.
33. Patrick S W, Dudley J, Martin P R, Harrell F E, Warren M D, Hartmann K E, Ely E W, Grijalva C G, Cooper W O. Prescription opioid epidemic and infant outcomes. Pediatrics 2015; 135:842-50.
34. Patrick S W, Schiff D M, Committee On Substance USE, Prevention. A Public Health Response to Opioid Use in Pregnancy. Pediatrics 2017; 139.
35. McQueen K, Murphy-Oikonen J. Neonatal Abstinence Syndrome. N Engl J Med 2016; 375:2468-79.
36. Feder K A, Letourneau E J, Brook J. Children in the Opioid Epidemic: Addressing the Next Generation's Public Health Crisis. Pediatrics 2019; 143.
37. Hudak M L, Tan R C, Committee On D, Committee On F, Newborn, American Academy of P. Neonatal drug withdrawal. Pediatrics 2012; 129:e540-60.
38. Hygiene MDoHaM. Maryland Maternal Mortality Review 2016 Annual Report. In: DHMH, ed. Baltimore, MD2016.
39. Jick H, Holmes L B, Hunter J R, Madsen S, Stergachis A. First-trimester drug use and congenital disorders. JAMA: the journal of the American Medical Association 1981; 246:343-6.
40. Zierler S, Rothman K J. Congenital heart disease in relation to maternal use of Bendectin and other drugs in early pregnancy. The New England journal of medicine 1985; 313:347-52.
41. Broussard C S, Rasmussen S A, Reefhuis J, Friedman J M, Jann M W, Riehle-Colarusso T, Honein M A, National Birth Defects Prevention S. Maternal treatment with opioid analgesics and risk for birth defects. American journal of obstetrics and gynecology 2011; 204:314 e1-11.
42. Shaw G M, Malcoe L H, Swan S H, Cummins S K, Schulman J. Congenital cardiac anomalies relative to selected maternal exposures and conditions during early pregnancy. Eur J Epidemiol 1992; 8:757-60.
43. Nezvalova-Henriksen K, Spigset O, Nordeng H. Effects of codeine on pregnancy outcome: results from a large population-based cohort study. Eur J Clin Pharmacol 2011; 67:1253-61.
44. Sirnes E, Oltedal L, Bartsch H, Eide G E, Elgen I B, Aukland S M. Brain morphology in school-aged children with prenatal opioid exposure: A structural MRI study. Early human development 2017; 106-107:33-9.
45. Monnelly V J, Anblagan D, Quigley A, Cabez M B, Cooper E S, Mactier H, Semple S I, Bastin M E, Boardman J P. Prenatal methadone exposure is associated with altered neonatal brain development. NeuroImage Clinical 2018; 18:9-14.
46. Monnelly V J, Hamilton R, Chappell F M, Mactier H, Boardman J P. Childhood neurodevelopment after prescription of maintenance methadone for opioid dependency in pregnancy: a systematic review and metaanalysis. Developmental medicine and child neurology 2018.
47. Sundelin Wahlsten V, Sarman I. Neurobehavioural development of preschool-age children born to addicted mothers given opiate maintenance treatment with buprenorphine during pregnancy. Acta paediatrica 2013; 102:544-9.
48. Monnelly V J, Hamilton R, Chappell F M, Mactier H, Boardman J P. Childhood neurodevelopment after prescription of maintenance methadone for opioid dependency in pregnancy: a systematic review and metaanalysis. Developmental medicine and child neurology 2019; 61:750-60.
49. Patrick S W, Davis M M, Lehmann C U, Cooper W O. Increasing incidence and geographic distribution of neonatal abstinence syndrome: United States 2009 to 2012. J Perinatol 2015; 35:650-5.
50. Schiff D M, Patrick S W. Treatment of Opioid Use Disorder During Pregnancy and Cases of Neonatal Abstinence Syndrome. JAMA pediatrics 2017.
51. Brandt L, Finnegan L P. Neonatal abstinence syndrome: where are we, and where do we go from here? Curr Opin Psychiatry 2017.
52. Conradt E, Flannery T, Aschner J L, Annett R D, Croen L A, Duarte C S, Friedman A M, Guille C, Hedderson M M, Hofheimer J A, Jones M R, Ladd-Acosta C, McGrath M, Moreland A, Neiderhiser J M, Nguyen R H N, Posner J, Ross J L, Savitz D A, Ondersma S J, Lester B M. Prenatal Opioid Exposure:Neurodevelopmental Consequences and Future Research Priorities. Pediatrics 2019; 144.
53. Reddy U M, Davis J M, Ren Z, Greene M F, Opioid Use in Pregnancy NAS, Childhood Outcomes Workshop Invited S. Opioid Use in Pregnancy, Neonatal Abstinence Syndrome, and Childhood Outcomes: Executive Summary of a Joint Workshop by the Eunice Kennedy Shriver National Institute of Child Health and Human Development, American College of Obstetricians and Gynecologists, American Academy of Pediatrics, Society for Maternal-Fetal Medicine, Centers for Disease Control and Prevention, and the March of Dimes Foundation. Obstet Gynecol 2017; 130:10-28.
54. Oei J L, Melhuish E, Uebel H, Azzam N, Breen C, Burns L, Hilder L, Bajuk B, Abdel-Latif M E, Ward M, Feller J M, Falconer J, Clews S, Eastwood J, Li A, Wright I M. Neonatal Abstinence Syndrome and High School Performance. Pediatrics 2017; 139.
55. Younger J W, Chu L F, D'Arcy N T, Trott K E, Jastrzab L E, Mackey S C. Prescription opioid analgesics rapidly change the human brain. Pain 2011; 152:1803-10.
56. McLaughlin P J, Zagon I S, White W J. Perinatal methadone exposure in rats. Effects on body and organ development. Biol Neonate 1978; 34:48-54.
57. Zagon I S, McLaughlin P J. Methadone and brain development. Experientia 1977; 33:1486-7.
58. Zagon I S, McLaughlin P J. The effects of different schedules of methadone treatment on rat brain development. Experimental neurology 1977; 56:538-52.
59. Zagon I S, McLaughlin P J. Effect of chronic maternal methadone exposure on perinatal development. Biol Neonate 1977; 31:271-82.
60. Zagon I S, McLaughlin P J. Perinatal methadone exposure and brain development: a biochemical study. J Neurochem 1978; 31:49-54.
61. Wu V W, Mo Q, Yabe T, Schwartz J P, Robinson S E. Perinatal opioids reduce striatal nerve growth factor content in rat striatum. Eur J Pharmacol 2001; 414:211-4.
62. Robinson S E, Maher J R, Wallace M J, Kunko P M. Perinatal methadone exposure affects dopamine, norepinephrine, and serotonin in the weanling rat. Neurotoxicol Teratol 1997; 19:295-303.
63. Nekhayeva I A, Nanovskaya T N, Deshmukh S V, Zharikova O L, Hankins G D, Ahmed M S. Bidirectional transfer of methadone across human placenta. Biochem Pharmacol 2005; 69:187-97.
64. Gerdin E, Rane A, Lindberg B. Transplacental transfer of morphine in man. J Perinat Med 1990; 18:305-12.
65. Jansson L M, Velez M, McConnell K, Spencer N, Tuten M, Jones H E, King V L, Gandotra N, Milio L A, Voegtline K, DiPietro J A. Maternal buprenorphine treatment and fetal neurobehavioral development. American journal of obstetrics and gynecology 2017; 216:529 e1-e8.
66. Jansson L M, Velez M L, McConnell K, Spencer N, Tuten M, Jones H, Rios R, King V L, Gandotra N, Millio L, DiPietro J A. Maternal buprenorphine treatment and infant outcome. Drug Alcohol Depend 2017; 180:56-61.
67. Velez M L, McConnell K, Spencer N, Montoya L, Tuten M, Jansson L M. Prenatal buprenorphine exposure and neonatal neurobehavioral functioning. Early human development 2018; 117:7-14.
68. Jansson L M, Dipietro J A, Velez M, Elko A, Williams E, Milio L, O'Grady K, Jones H E. Fetal neurobehavioral effects of exposure to methadone or buprenorphine. Neurotoxicol Teratol 2011; 33:240-3.
69. Kaltenbach K, O'Grady K E, Heil S H, Salisbury A L, Coyle M G, Fischer G, Martin P R, Stine S, Jones H E. Prenatal exposure to methadone or buprenorphine: Early childhood developmental outcomes. Drug Alcohol Depend 2018; 185:40-9.
70. Bakhireva L N, Holbrook B D, Shrestha S, Leyva Y, Ashley M, Cano S, Lowe J, Stephen J M, Leeman L. Association between prenatal opioid exposure, neonatal opioid withdrawal syndrome, and neurodevelopmental and behavioral outcomes at 5-8 months of age. Early human development 2019; 128:69-76.
71. Jansson L M, Di Pietro J A, Elko A, Williams E L, Milio L, Velez M. Pregnancies exposed to methadone, methadone and other illicit substances, and poly-drugs without methadone: a comparison of fetal neurobehaviors and infant outcomes. Drug Alcohol Depend 2012; 122:213-9.
72. Mazur M, Miller R H, Robinson S. Postnatal erythropoietin treatment mitigates neural cell loss after systemic prenatal hypoxic-ischemic injury. Journal of neurosurgery Pediatrics 2010; 6:206-21.
73. Bussey T J, Holmes A, Lyon L, Mar A C, McAllister K A, Nithianantharajah J, Oomen C A, Saksida L M. New translational assays for preclinical modelling of cognition in schizophrenia: the touchscreen testing method for mice and rats. Neuropharmacology 2012; 62:1191-203.
74. Bussey T J, Padain T L, Skillings E A, Winters B D, Morton A J, Saksida L M. The touchscreen cognitive testing method for rodents: how to get the best out of your rat. Learn Mem 2008; 15:516-23.
75. Horner A E, Heath C J, Hvoslef-Eide M, Kent B A, Kim C H, Nilsson S R, Alsio J, Oomen C A, Holmes A, Saksida L M, Bussey T J. The touchscreen operant platform for testing learning and memory in rats and mice. Nature protocols 2013; 8:1961-84.
76. Nithianantharajah J, Grant S G. Cognitive components in mice and humans: combining genetics and touchscreens for medical translation. Neurobiology of learning and memory 2013; 105:13-9.
77. Oomen C A, Hvoslef-Eide M, Heath C J, Mar A C, Homer A E, Bussey T J, Saksida L M. The touchscreen operant platform for testing working memory and pattern separation in rats and mice. Nature protocols 2013; 8:2006-21.
78. Herman A P, Bochenek J, Krol K, Krawczynska A, Antushevich H, Pawlina B, Herman A, Romanowicz K, Tomaszewska-Zaremba D. Central Interleukin-1beta Suppresses the Nocturnal Secretion of Melatonin. Mediators Inflamm 2016; 2016:2589483.
79. Mazur M, Miller R, Robinson S. Postnatal erythropoietin treatment mitigates neural cell loss after systemic prenatal hypoxic-ischemic injury. J Neurosurg Pediatrics 2010; 6:206-21.
80. Robinson S, Conteh F, Oppong A, Yellowhair T, Newville J, El Demerdash N, Shrock C, Maxwell J, Jett S, Northington F, Jantzie L. Extended combined neonatal treatment with erythropoietin plus melatonin prevents posthemorrhagic hydrocephalus of prematurity in rats. Front Cell Neurosci 2018; 12:322.
81. Jantzie L L, Winer J L, Corbett C J, Robinson S. Erythropoietin Modulates Cerebral and Serum Degradation Products from Excess Calpain Activation following Prenatal Hypoxia-Ischemia. Developmental neuroscience 2016; 38:15-26.
82. Volpe J J. Dysmaturation of Premature Brain: Importance, Cellular Mechanisms, and Potential Interventions. Pediatric neurology 2019; 95:42-66.
83. Wang R, Li J, Duan Y, Tao Z, Zhao H, Luo Y. Effects of Erythropoietin on Gliogenesis during Cerebral Ischemic/Reperfusion Recovery in Adult Mice. Aging Dis 2017; 8:410-9.
84. McDougall A R A, Hale N, Rees S, Harding R, De Matteo R, Hooper S B, Tolcos M. Erythropoietin Protects Against Lipopolysaccharide-Induced Microgliosis and Abnormal Granule Cell Development in the Ovine Fetal Cerebellum. Front Cell Neurosci 2017; 11:224.
85. Dominguez Rubio A P, Correa F, Aisemberg J, Dorfman D, Bariani M V, Rosenstein R E, Zorrilla Zubilete M, Franchi A M. Maternal administration of melatonin exerts short- and long-term neuroprotective effects on the offspring from lipopolysaccharide-treated mice. Journal of pineal research 2017.
86. Man G C W, Zhang T, Chen X, Wang J, Wu F, Liu Y, Wang C C, Cheong Y, Li T C. The regulations and role of circadian clock and melatonin in uterine receptivity and pregnancy—An immunological perspective. American journal of reproductive immunology 2017; 78.
87. Kallapur S G, Presicce P, Rueda C M, Jobe A H, Chougnet C A. Fetal immune response to chorioamnionitis. Semin Reprod Med 2014; 32:56-67.
88. Yellowhair T R, Noor S, Mares B, Jose C, Newville J, Maxwell J, Northington F, Milligan E D, Robinson S, Jantzie L L. Chorioamnionitis in Rats Precipitates Extended Postnatal Inflammatory Lymphocyte Hyper-Reactivity. Dev Neurosci 2019 Mar. 28:1-11.
89. Juul S. Neuroprotective role of erythropoietin in neonates. The journal of maternal-fetal & neonatal medicine: the official journal of the European Association of Perinatal Medicine, the Federation of Asia and Oceania Perinatal Societies, the International Society of Perinatal Obstet 2012; 25 Suppl 4:105-7.
90. Miskowiak K W, Vinberg M, Glerup L, Paulson O B, Knudsen G M, Ehrenreich H, Harmer C J, Kessing L V, Siebner H R, Macoveanu J. Neural correlates of improved executive function following erythropoietin treatment in mood disorders. Psychol Med 2016; 46:1679-91.
91. Ehrenreich H, Hinze-Selch D, Stawicki S, Aust C, Knolle-Veentjer S, Wilms S, Heinz G, Erdag S, Jahn H, Degner D, Ritzen M, Mohr A, Wagner M, Schneider U, Bohn M, Huber M, Czernik A, Pollmacher T, Maier W, Siren A L, Klosterkotter J, Falkai P, Ruther E, Aldenhoff J B, Krampe H. Improvement of cognitive functions in chronic schizophrenic patients by recombinant human erythropoietin. Mol Psychiatry 2007; 12:206-20.
92. Jantzie L L, Miller R H, Robinson S. Erythropoietin signaling promotes oligodendrocyte development following prenatal systemic hypoxic-ischemic brain injury. Pediatric research 2013; 74:658-67.
93. Li Z, Li X, Chan M T V, Wu W K K, Tan D, Shen J. Melatonin antagonizes interleukin-18-mediated inhibition on neural stem cell proliferation and differentiation. J Cell Mol Med 2017; 21:2163-71.
94. Xue F, Shi C, Chen Q, Hang W, Xia L, Wu Y, Tao S Z, Zhou J, Shi A, Chen J. Melatonin Mediates Protective Effects against Kainic Acid-Induced Neuronal Death through Safeguarding E R Stress and Mitochondrial Disturbance. Frontiers in molecular neuroscience 2017; 10:49.
95. Das A, Wallace Gt, Reiter R J, Varma A K, Ray S K, Banik N L. Overexpression of melatonin membrane receptors increases calcium-binding proteins and protects VSC4.1 motoneurons from glutamate toxicity through multiple mechanisms. Journal of pineal research 2013; 54:58-68.
96. Zhao H, Wang R, Wu X, Liang J, Qi Z, Liu X, Min L, Ji X, Luo Y. Erythropoietin delivered via intraarterial infusion reduces endoplasmic reticulum stress in brain microvessels of rats following cerebral ischemia and reperfusion. J Neuroimmune Pharmacol 2015; 10:153-61.
97. Hong Z, Hong H, Chen H, Wang Z, Hong D. Protective effects of erythropoietin in experimental spinal cord injury by reducing the C/EBP-homologous protein expression. Neurol Res 2012; 34:85-90.
98. Lu J, Dai Q M, Ma G S, Zhu Y H, Chen B, Li B, Yao Y Y. Erythropoietin Attenuates Cardiac Dysfunction in Rats by Inhibiting Endoplasmic Reticulum Stress-Induced Diabetic Cardiomyopathy. Cardiovase Drugs Ther 2017.
99. Feng D, Wang B, Wang L, Abraham N, Tao K, Huang L, Shi W, Dong Y, Qu Y. Pre-ischemia melatonin treatment alleviated acute neuronal injury after ischemic stroke by inhibiting endoplasmic reticulum stress dependent autophagy via PERK and IRE1 signalings. Journal of pineal research 2017; 62.
100. Fernandez A, Ordonez R, Reiter R J, Gonzalez-Gallego J, Mauriz J L. Melatonin and endoplasmic reticulum stress: relation to autophagy and apoptosis. J Pineal Res 2015; 59:292-307.
101. Hadj Ayed Tka K, Mahfoudh Boussaid A, Zaouali M A, Kammoun R, Bejaoui M, Ghoul Mazgar S, Rosello Catafau J, Ben Abdennebi H. Melatonin modulates endoplasmic reticulum stress and Akt/GSK3-beta signaling pathway in a rat model of renal warm ischemia reperfusion. Anal Cell Pathol (Amst) 2015; 2015:635172.
102. Hardeland R. Melatonin and the electron transport chain. Cellular and molecular life sciences: CMLS 2017.
103. Mendivil-Perez M, Soto-Mercado V, Guerra-Librero A, Fernandez-Gil B I, Florido J, Shen Y Q, Tejada M A, Capilla-Gonzalez V, Rusanova I, Garcia-Verdugo J M, Acuna-Castroviejo D, Lopez L C, Velez-Pardo C, Jimenez-Del-Rio M, Ferrer J M, Escames G. Melatonin enhances neural stem cell differentiation and engraftment by increasing mitochondrial function. Journal of pineal research 2017; 63.
104. Ng K Y, Leong M K, Liang H, Paxinos G. Melatonin receptors: distribution in mammalian brain and their respective putative functions. Brain structure & function 2017.
105. Hu W, Ma Z, Di S, Jiang S, Li Y, Fan C, Yang Y, Wang D. Snapshot: implications for melatonin in endoplasmic reticulum homeostasis. Br J Pharmacol 2016; 173:3431-42.
106. Fisher J W. Landmark advances in the development of erythropoietin. Experimental biology and medicine 2010; 235:1398-411.
107. Zanjani E D, Poster J, Burlington H, Mann L I, Wasserman L R. Liver as the primary site of erythropoietin formation in the fetus. J Lab Clin Med 1977; 89:640-4.
108. D'Andrea A D, Lodish H F, Wong G G. Expression cloning of the murine erythropoietin receptor. Cell 1989; 57:277-85.
109. Yu X, Shacka J J, Eells J B, Suarez-Quian C, Przygodzki R M, Beleslin-Cokic B, Lin C S, Nikodem V M, Hempstead B, Flanders K C, Costantini F, Noguchi C T. Erythropoietin receptor signalling is required for normal brain development. Development 2002; 129:505-16.
110. Digicaylioglu M, Bichet S, Matrti H, Wenger R, Rivas L, Bauer C, Gassmann M. Localization of specific erythropoietin binding sites in defined areas of the mouse brain. Proc Natl Acad Sci 1995; 92:3737-20.
111. Masuda S, Nagao M, Takahata K, Konishi Y, Gallyas F, Jr., Tabira T, Sasaki R. Functional erythropoietin receptor of the cells with neural characteristics. Comparison with receptor properties of erythroid cells. J Biol Chem 1993; 268:11208-16.
112. Ott C, Martens H, Hassouna I, Oliveira B, Erck C, Zafeiriou M P, Peteri U K, Hesse D, Gerhart S, Altas B, Kolbow T, Stadler H, Kawabe H, Zimmermann W H, Nave K A, Schulz-Schaeffer W, Jahn O, Ehrenreich H. Widespread expression of erythropoietin receptor in brain and its induction by injury. Mol Med 2015.
113. Sugawa M, Sakurai Y, Ishikawa-Ieda Y, Suzuki H, Asou H. Effects of erythropoietin on glial cell development: Oligodendrocyte maturation and astrocyte proliferation. Neurosci Res 2002; 44:391-403.
114. Bernaudin M, Harti H, Roussel S, Divoux D, Nouvelot A, MacKenzie E, Petit E. A potential role for erythropoietin in focal permanent cerebral ischemia in mice. J Cerebral Blood Flow Metab 1999; 19:643-51.
115. Marti H. Erythropoietin and the hypoxic brain. The Journal of Experimental Biology 2004; 207:3233-42.
116. Chavez J, Baranova O, Lin J, Pichiule P. The transcriptional activator hypoxia inducible factor 2 (HIF-2/EPAS-1) regulates the oxygen-dependent expression of erythropoietin in cortical astrocytes. J Neurosci 2006; 26:9471-81.
117. Sanchez P E, Navarro F P, Fares R P, Nadam J, Georges B, Moulin C, Le Cavorsin M, Bonnet C, Ryvlin P, Belmeguenai A, Bodennec J, Morales A, Bezin L. Erythropoietin receptor expression is concordant with erythropoietin but not with common beta chain expression in the rat brain throughout the life span. J Comp Neurol 2009; 514:403-14.
118. Knabe W, Knerlich F, Washausen S, Kietzmann T, Siren A, Brunnett G, Kuhn H, Ehrenreich H. Expression patterns of erythropoietin and its receptor in the developing midbrain. Anat Embryol 2004; 207:503-12.
119. Knabe W, Siren A-L, Ehrenreigh H, Kuhn H-J. Expression patterns of erythropoietin and its receptor in the developing spinal cord and dorsal root ganglia. Anat Embryol 2005; 210:209-19.
120. Ransome M I, Turnley A M. Systemically delivered Erythropoietin transiently enhances adult hippocampal neurogenesis. Journal of neurochemistry 2007; 102:1953-65.
121. Iwai M, Cao G, Yin W, Stetler R A, Liu J, Chen J. Erythropoietin promotes neuronal replacement through revascularization and neurogenesis after neonatal hypoxia/ischemia in rats. Stroke 2007; 38:2795-803.
122. Iwai M, Stetler R A, Xing J, Hu X, Gao Y, Zhang W, Chen J, Cao G. Enhanced oligodendrogenesis and recovery of neurological function by erythropoietin after neonatal hypoxic/ischemic brain injury. Stroke 2010; 41:1032-7.
123. Onaolapo A Y, Aina O A, Onaolapo O J. Melatonin attenuates behavioural deficits and reduces brain oxidative stress in a rodent model of schizophrenia. Biomed Pharmacother 2017; 92:373-83.
124. Venegas C, Garcia J A, Escames G, Ortiz F, Lopez A, Doerrier C, Garcia-Corzo L, Lopez L C, Reiter R J, Acuna-Castroviejo D. Extrapineal melatonin: analysis of its subcellular distribution and daily fluctuations. J Pineal Res 2012; 52:217-27.
125. Ekmekcioglu C. Melatonin receptors in humans: biological role and clinical relevance. Biomed Pharmacother 2006; 60:97-108.
126. Binfare R W, Mantovani M, Budni J, Santos A R, Rodrigues A L. Involvement of doparine receptors in the antidepressant-like effect of melatonin in the tail suspension test. Eur J Pharmacol 2010; 638:78-83.
127. Mantovani M, Kaster M P, Pertile R, Calixto J B, Rodrigues A L, Santos A R. Mechanisms involved in the antinociception caused by melatonin in mice. J Pineal Res 2006; 41:382-9.
128. Mantovani M, Pertile R, Calixto J B, Santos A R, Rodrigues A L. Melatonin exerts an antidepressant-like effect in the tail suspension test in mice: evidence for involvement of N-methyl-D-aspartate receptors and the L-arginine-nitric oxide pathway. Neurosci Lett 2003; 343:1-4.
129. Berkiks I, Benmhammed H, Mesfioui A, Ouichou A, El Hasnaoui A, Mouden S, Touil T, Bahbiti Y, Nakache R, El Hessni A. Postnatal melatonin treatment protects against affective disorders induced by earlylife immune stimulation by reducing the microglia cell activation and oxidative stress. Int J Neurosci 2017:1-10.
130. Okatani Y, Okamoto K, Hayashi K, Wakatsuki A, Tamura S, Sagara Y. Maternal-fetal transfer of melatonin in pregnant women near term. J Pineal Res 1998; 25:129-34.
131. Szczepanik M. Melatonin and its influence on immune system. J Physiol Pharmacol 2007; 58 Suppl 6:115-24.
132. Teramo K A, Klemetti M M, Widness J A. Robust increases in erythropoietin production by the hypoxic fetus is a response to protect the brain and other vital organs. Pediatric research 2018.
133. Logan J W, Allred E N, Fichorova R N, Engelke S, Dammann O, Leviton A, Investigators E S. Endogenous erythropoietin varies significantly with inflammation-related proteins in extremely premature newborns. Cytokine 2014; 69:22-8.
134. Teramo K A, Widness J A. Increased fetal plasma and amniotic fluid erythropoietin concentrations: markers of intrauterine hypoxia. Neonatology 2009; 95:105-16.
135. Sweetman D U, Onwuneme C, Watson W R, Murphy J F, Molloy E J. Perinatal Asphyxia and Erythropoietin and VEGF: Serial Serum and Cerebrospinal Fluid Responses. Neonatology 2017; 111:253-9.
136. Assaraf M I, Diaz Z, Liberman A, Miller W H, Jr., Arvanitakis Z, Li Y, Bennett D A, Schipper H M. Brain erythropoietin receptor expression in Alzheimer disease and mild cognitive impairment. J Neuropathol Exp Neurol 2007; 66:389-98.
137. Robinson S, Winer J L, Berkner J, Chan L A, Denson J L, Maxwell J R, Yang Y, Sillerud L O, Tasker R C, Meehan W P, 3rd, Mannix R, Jantzie L L. Imaging and serum biomarkers reflecting the functional efficacy of extended erythropoietin treatment in rats following infantile traumatic brain injury. Journal of neurosurgery Pediatrics 2016:1-17.
138. Keller M, Yang J, Griesmaier E, Gorna A, Sarkozy G, Urbanek M, Gressens P, Simbruner G. Erythropoietin is neuroprotective against NMDA-receptor-mediated excitotoxic brain injury in newborn mice. Neurobiol Dis 2006; 24:357-66.
139. Fauchere J C, Koller B M, Tschopp A, Dame C, Ruegger C, Bucher H U, Swiss Erythropoietin Neuroprotection Trial G. Safety of Early High-Dose Recombinant Erythropoietin for Neuroprotection in Very Preterm Infants. The Journal of pediatrics 2015; 167:52-7 e3.
140. Wu Y W, Mathur A M, Chang T, McKinstry R C, Mulkey S B, Mayock D E, Van Meurs K P, Rogers E E, Gonzalez F F, Comstock B A, Juul S E, Msall M E, Bonifacio S L, Glass H C, Massaro A N, Dong L, Tan K W, Heagerty P J, Ballard R A. High-Dose Erythropoietin and Hypothermia for Hypoxic-Ischemic Encephalopathy: A Phase II Trial. Pediatrics 2016; 137:e20160191.
141. Ohls R K, Cannon D C, Phillips J, Caprihan A, Patel S, Winter S, Steffen M, Yeo R A, Campbell R, Wiedmeier S, Baker S, Gonzales S, Lowe J. Preschool Assessment of Preterm Infants Treated With Darbepoetin and Erythropoietin. Pediatrics 2016; 137:e20153859.
142. Ohls R K, Kamath-Rayne B D, Christensen R D, Wiedmeier S E, Rosenberg A, Fuller J, Lacy C B, Roohi M, Lambert D K, Burnett J J, Pruckler B, Peceny H, Cannon D C, Lowe J R. Cognitive outcomes of preterm infants randomized to darbepoetin, erythropoietin, or placebo. Pediatrics 2014; 133:1023-30.
143. Carloni S, Proietti F, Rocchi M, Longini M, Marseglia L, D'Angelo G, Balduini W, Gitto E, Buonocore G Melatonin Pharmacokinetics Following Oral Administration in Preterm Neonates. Molecules 2017; 22:E2115.
144. Juul S, McPherson R, Bauer L, Ledbetter K, Gleason C, Mayock D. A phase I/II trial of high-dose erythropoietin in extremely low birth weight infants: pharmacokinetics and safety. Pediatrics 2008; 122:383-91.
145. Song J, Sun H, Xu F, Kang W, Gao L, Guo J, Zhang Y, Xia L, Wang X, Zhu C. Recombinant human erythropoietin improves neurological outcomes in very preterm infants. Ann Neurol 2016; 80:24-34.
146. Fauchere J-C, Dame C, Vonthein R, Koller B, Arri S, Wolf M, Bucher H. An approach to using recombinant erythropoietin for neuroprotection in very preterm infants. Pediatrics 2008; 122:375-82.
147. Benders M J, van der Aa N E, Roks M, van Straaten H L, Isgum I, Viergever M A, Groenendaal F, de Vries L S, van Bel F. Feasibility and safety of erythropoietin for neuroprotection after perinatal arterial ischemic stroke. The Journal of pediatrics 2014; 164:481-6 e1-2.
148. Gonzalez F F, Ferriero D M. Therapeutics for neonatal brain injury. Pharmacol Ther 2008; 120:43-53.
149. Lin C Y, Chang Y C, Wang S T, Lee T Y, Lin C F, Huang C C. Altered inflammatory responses in preterm children with cerebral palsy. Annals of neurology 2010; 68:204-12.
150. Semple B D, Blomgren K, Gimlin K, Ferriero D M, Noble-Haeusslein U. Brain development in rodents and humans: Identifying benchmarks of maturation and vulnerability to injury across species. Progress in neurobiology 2013; 106-107:1-16.
151. Zedler B K, Mann A L, Kim M M, Amick H R, Joyce A R, Murrelle E L, Jones H E. Buprenorphine compared with methadone to treat pregnant women with opioid use disorder: a systematic review and meta-analysis of safety in the mother, fetus and child. Addiction 2016; 111:2115-28.
152. Mactier H, Shipton D, Dryden C, Tappin D M. Reduced fetal growth in methadone-maintained pregnancies is not fully explained by smoking or socio-economic deprivation. Addiction 2014; 109:482-8.
153. Wouldes T A, Woodward U. Maternal methadone dose during pregnancy and infant clinical outcome. Neurotoxicol Teratol 2010; 32:406-13.
154. Brigman J L, Daut R A, Wright T, Gunduz-Cinar O, Graybeal C, Davis M I, Jiang Z, Saksida L M, Jinde S, Pease M, Bussey T J, Lovinger D M, Nakazawa K, Holmes A. GluN2B in corticostriatal circuits governs choice learning and choice shifting. Nat Neurosci 2013; 16:1101-10.
155. Marquardt K, Sigdel R, Caldwell K, Brigman J L. Prenatal ethanol exposure impairs executive function in mice into adulthood. Alcoholism, clinical and experimental research 2014; 38:2962-8.
156. Marquardt K, Josey M, Kenton J A, Cavanagh J F, Holmes A, Brigman J L. Impaired cognitive flexibility following NMDAR-GluN2B deletion is associated with altered orbitofrontal-striatal function. Neuroscience 2019; 404:338-52.
157. Bergstrom H C, Lipkin A M, Lieberman A G, Pinard C R, Gunduz-Cinar O, Brockway E T, Taylor W W, Nonaka M, Bukalo O, Wills T A, Rubio F J, Li X, Pickens C L, Winder D G, Holmes A. Dorsolateral Striatum Engagement Interferes with Early Discrimination Learning. Cell Rep 2018; 23:2264-72.
158. Yeoh S L, Eastwood J, Wright I M, Morton R, Melhuish E, Ward M, Oei J L. Cognitive and Motor Outcomes of Children With Prenatal Opioid Exposure: A Systematic Review and Meta-analysis. JAMA Netw Open 2019; 2:e197025.
159. Nygaard E, Slinning K, Moe V, Walhovd K B. Cognitive function of youths born to mothers with opioid and poly-substance abuse problems during pregnancy. Child Neuropsychol 2017; 23:159-87.
160. Konijnenberg C, Melinder A. Executive function in preschool children prenatally exposed to methadone or buprenorphine. Child Neuropsychol 2015; 21:570-85.
161. Nygaard E, Moe V, Slinning K, Walhovd K B. Longitudinal cognitive development of children born to mothers with opioid and polysubstance use. Pediatric research 2015; 78:330-5.
162. Nygaard E, Slinning K, Moe V, Due-Tonnessen P, Fjell A, Walhovd K B. Neuroanatomical characteristics of youths with prenatal opioid and poly-drug exposure. Neurotoxicol Teratol 2018; 68:13-26.
163. Wehrle F M, Kaufmann L, Benz L D, Huber R, O'Gorman R L, Latal B, Hagmann C F. Very preterm adolescents show impaired performance with increasing demands in executive function tasks. Early human development 2016; 92:37-43.
164. Kocek M, Wilcox R, Crank C, Patra K. Evaluation of the relationship between opioid exposure in extremely low birth weight infants in the neonatal intensive care unit and neurodevelopmental outcome at 2 years. Early human development 2016; 92:29-32.
165. McGlone L, Mactier H. Infants of opioid-dependent mothers: neurodevelopment at six months. Early human development 2015; 91:19-21.
166. Li W, Li Q, Wang Y, Zhu J, Ye J, Yan X, Li Y, Chen J, Liu J, Li Z, Wang W, Liu Y. Methadone-induced Damage to White Matter Integrity in Methadone Maintenance Patients: A Longitudinal Self-control DTI Study. Sci Rep 2016; 6:19662.
167. Walhovd K B, Watts R, Amlien I, Woodward U. Neural tract development of infants born to methadonemaintained mothers. Pediatric neurology 2012; 47:1-6.
168. Murner-Lavanchy I M, Kidokoro H, Thompson D K, Doyle L W, Cheong J L Y, Hunt R W, Inder T E, Anderson P J. Thirteen-Year Outcomes in Very Preterm Children Associated with Diffuse Excessive High Signal Intensity on Neonatal Magnetic Resonance Imaging. The Journal of pediatrics 2019; 206:66-71 e1.
169. Kelly C E, Chan L, Burnett A C, Lee K J, Connelly A, Anderson P J, Doyle L W, Cheong J L, Thompson D K, Victorian Infant Collaborative Study G. Brain structural and microstructural alterations associated with cerebral palsy and motor impairments in adolescents born extremely preterm and/or extremely low birthweight. Dev Med Child Neurol 2015; 57:1168-75.
170. Lean R E, Han R H, Smyser T A, Kenley J K, Shimony J S, Rogers C E, Limbrick D D, Jr., Smyser C D. Altered neonatal white and gray matter microstructure is associated with neurodevelopmental impairments in very preterm infants with high-grade brain injury. Pediatric research 2019; 86:365-74.
171. Vestal-Laborde A A, Eschenroeder A C, Bigbee J W, Robinson S E, Sato-Bigbee C. The opioid system and brain development: effects of methadone on the oligodendrocyte lineage and the early stages of myelination. Developmental neuroscience 2014; 36:409-21.
172. Sanchez E S, Bigbee J W, Fobbs W, Robinson S E, Sato-Bigbee C. Opioid addiction and pregnancy:perinatal exposure to buprenorphine affects myelination in the developing brain. Glia 2008; 56:1017-27.
173. Maxwell J R, Denson J L, Joste N E, Robinson S, Jantzie L L. Combined in utero hypoxia-ischemia and lipopolysaccharide administration in rats induces chorioamnionitis and a fetal inflammatory response syndrome. Placenta 2015; 36:1378-84.
174. Madsen-Bouterse S A, Romero R, Tarca A L, Kusanovic J P, Espinoza J, Kim C J, Kim J S, Edwin S S, Gomez R, Draghici S. The transcriptome of the fetal inflammatory response syndrome. American journal of reproductive immunology 2010; 63:73-92.
175. Gomez R, Romero R, Ghezzi F, Yoon B H, Mazor M, Berry S M. The fetal inflammatory response syndrome. American journal of obstetrics and gynecology 1998; 179:194-202.
176. Romero R, Gomez R, Ghezzi F, Yoon B H, Mazor M, Edwin S S, Berry S M. A fetal systemic inflammatory response is followed by the spontaneous onset of preterm parturition. American journal of obstetrics and gynecology 1998; 179:186-93.
177. Galinsky R, Polglase G R, Hooper S B, Black M J, Moss T J. The consequences of chorioamnionitis: preterm birth and effects on development. Journal of pregnancy 2013; 2013:412831.
178. Andrews W W, Goldenberg R L, Faye-Petersen O, Cliver S, Goepfert A R, Hauth J C. The Alabama Preterm Birth study: polymorphonuclear and mononuclear cell placental infiltrations, other markers of inflammation, and outcomes in 23- to 32-week preterm newborn infants. American journal of obstetrics and gynecology 2006; 195:803-8.
179. Gotsch F, Romero R, Kusanovic J P, Mazaki-Tovi S, Pineles B L, Erez O, Espinoza J, Hassan S S. The fetal inflammatory response syndrome. Clinical obstetrics and gynecology 2007; 50:652-83.
180. Dammann O, Allred E N, Fichorova R N, Kuban K, O'Shea T M, Leviton A, Investigators E S. Duration of Systemic Inflammation in the First Postnatal Month Among Infants Born Before the 28th Week of Gestation Inflammation 2016; 39:672-7.
181. Dowling O, Chatterjee P K, Gupta M, Tam Tam H B, Xue X, Lewis D, Rochelson B, Metz C N. Magnesium sulfate reduces bacterial LPS-induced inflammation at the maternal-fetal interface. Placenta 2012; 33:392-8.
182. O'Shea T M, Allred E N, Kuban K C, Dammann O, Paneth N, Fichorova R, Hirtz D, Leviton A, Extremely Low Gestational Age Newborn Study I. Elevated concentrations of inflammation-related proteins in postnatal blood predict severe developmental delay at 2 years of age in extremely preterm infants. J Pediatr 2012; 160:395-401 e4.
183. O'Shea T M, Joseph R M, Kuban K C, Allred E N, Ware J, Coster T, Fichorova R N, Dammann O, Leviton A, Investigators E S. Elevated blood levels of inflammation-related proteins are associated with an attention problem at age 24 mo in extremely preterm infants. Pediatric research 2014; 75:781-7.
184. Watterberg K L, Demers L M, Scott S M, Murphy S. Chorioamnionitis and early lung inflammation in infants in whom bronchopulmonary dysplasia develops. Pediatrics 1996; 97:210-5.
185. Watterberg K L, Scott S M, Naeye R L. Chorioamnionitis, cortisol, and acute lung disease in very low birth weight infants. Pediatrics 1997; 99:E6.
186. Kuban K C, O'Shea T M, Allred E N, Fichorova R N, Heeren T, Paneth N, Hirtz D, Dammann O, Leviton A, Investigators E S. The breadth and type of systemic inflammation and the risk of adverse neurological outcomes in extremely low gestation newborns. Pediatric neurology 2015; 52:42-8.
187. Leviton A, Fichorova R, Yamamoto Y, Allred E N, Dammann O, Hecht J, Kuban K, McElrath T, O'Shea T M, Paneth N. Inflammation-related proteins in the blood of extremely low gestational age newborns. The contribution of inflammation to the appearance of developmental regulation. Cytokine 2011; 53:66-73.
188. Leviton A, Kuban K C, Allred E N, Fichorova R N, O'Shea T M, Paneth N, Investigators E S. Early postnatal blood concentrations of inflammation-related proteins and microcephaly two years later in infants born before the 28th post-menstrual week. Early human development 2011; 87:325-30.
189. Leviton A, Allred E N, Fichorova R N, Kuban K C, Michael O'Shea T, Dammann O, investigators Es. Systemic inflammation on postnatal days 21 and 28 and indicators of brain dysfunction 2 years later among children born before the 28th week of gestation. Early human development 2016; 93:25-32.
190. Huggard D, McGrane F, Lagan N, Roche E, Balfe J, Leahy T R, Franklin O, Moreno A,
Melo A M, Doherty D G, Molloy E J. Altered endotoxin responsiveness in healthy children with Down syndrome. BMC Immunol 2018; 19:31.
191. Claus C P, Tsuru-Aoyagi K, Adwanikar H, Walker B, Manvelyan H, Whetstone W, Noble-Haeusslein L J. Age is a determinant of leukocyte infiltration and loss of cortical volume after traumatic brain injury, Developmental neuroscience 2010; 32:454-65.
192. Jantzie L L, Corbett C J, Berglass J, Firl D J, Flores J, Mannix R, Robinson S. Complex pattern of interaction between in utero hypoxia-ischemia and intra-amniotic inflammation disrupts brain development and motor function. J Neuroinflammation 2014; 11:131.
193. Jantzie L L, Getsy P, Denson J L, Firl D J, Wilson C G, Robinson S. Prenatal hypoxia-ischemia induces potassium chloride cotransporter 2 loss and abnormalities in inhibitory tone. Front Cell Neurosci 2015; 3:347.
194. Robinson S, Corbett C J, Winer J L, Chan L A S, Maxwell J R, Anstine C V, Yellowhair T R, Andrews N A, Yang Y, Sillerud L O, Jantzie L L. Neonatal erythropoietin mitigates impaired gait, social interaction and diffusion tensor imaging abnormalities in a rat model of prenatal brain injury. Experimental neurology 2017; 302:1-13
195. Korzeniewski S J, Allred E, Logan J W, Fichorova R N, Engelke S, Kuban K C, O'Shea T M, Paneth N, Holm M, Dammann O, Leviton A, investigators Es. Elevated endogenous erythropoietin concentrations are associated with increased risk of brain damage in extremely preterm neonates. PloS one 2015; 10:e0115083.
196. Sabic D, Koenig J M. A perfect storm: fetal inflammation and the developing immune system. Pediatric research 2019.
197. Meng Y, Xiong Y, Mahmood A, Zhang Y, Qu C, Chopp M. Dose-dependent neurorestorative effects of delayed treatment of traumatic brain injury with recombinant human erythropoietin in rats. J Neurosurg 2011; 115:550-60.
198. Graybeal C, Feyder M, Schulman E, Saksida L M, Bussey T J, Brigman J L, Holmes A. Paradoxical reversal learning enhancement by stress or prefrontal cortical damage: rescue with BDNF. Nat Neurosci 2011; 14:1507-9.
199. Jantzie L L, Talos D M, Selip D B, An L, Jackson M C, Folkerth R D, Deng W, Jensen F E. Developmental regulation of group I metabotropic glutamate receptors in the premature brain and their protective role in a rodent model of periventricular leukomalacia. Neuron Glia Biol 2010; 6:277-88.
200. Robinson S, Petelenz K, Li Q, Cohen M L, Dechant A, Tabrizi N, Bucek M, Lust D, Miller R H. Developmental changes induced by graded prenatal systemic hypoxic-ischemic insults in rats. Neurobiology of disease 2005; 18:568-81.
201. Jantzie L L, Hu M Y, Park H K, Jackson M C, Yu J, Maxwell J R, Jensen F E. Chloride cotransporter NKCC1 inhibitor bumetanide protects against white matter injury in a rodent model of periventricular leukomalacia. Pediatric research 2015; 77:554-62.
202. Robinson S, Berglass J, Denson J L, Berkner J, Anstine C V, Winer J L, Maxwell J, Qiu J, Yang Y, Sillerud, L, Meehan W P, Mannix R, Jantzie L L. Microstructural and microglial changes after repetitive mild traumatic brain injury in mice. J Neurosci Res 2017; 95(4):1025-1035.
203. Benoit J S, Hannay H J, Yamal J M, Francis D J, Aisiku I, Robertson C. Longitudinal Changes in Disability Rating Scale Scores: A Secondary Analysis Among Patients With Severe TBI Enrolled in the Epo Clinical Trial. Journal of the International Neuropsychological Society: JINS 2019; 25:293-301.
204. French C J, Glassford N J, Gantner D, Higgins A M, Cooper D J, Nichol A, Skrifvars M B, Imberger G, Presneill J, Bailey M, Bellomo R. Erythropoiesis-stimulating Agents in Critically Ill Trauma Patients: A Systematic Review and Meta-analysis. Ann Surg 2017; 265:54-62.
205. Liu W C, Wen L, Xie T, Wang H, Gong J B, Yang X F. Therapeutic effect of erythropoietin in patients with traumatic brain injury: a meta-analysis of randomized controlled trials. J Neurosurg 2017; 127:8-15.
206. Li Z M, Xiao Y L, Zhu J X, Geng F Y, Guo C J, Chong Z L, Wang L X. Recombinant human erythropoietin improves functional recovery in patients with severe traumatic brain injury: A randomized, double blind and controlled clinical trial. Clin Neurol Neurosurg 2016; 150:80-3.
207. Lee J, Cho Y, Choi K S, Kim W, Jang B H, Shin H, Ahn C, Lim T H, Yi H J. Efficacy and safety of erythropoietin in patients with traumatic brain injury: A systematic review and meta-analysis. Am J Emerg Med 2018.
208. Nichol A, French C, Little L, Haddad S, Presneill J, Arabi Y, Bailey M, Cooper D J, Duranteau J, Huet O, Mak A, McArthur C, Pettila V, Skrifvars M, Vallance S, Varma D, Wills J, Bellomo R, Investigators E-T, Group ACT. Erythropoietin in traumatic brain injury (EPO-TBI): a double-blind randomised controlled trial. Lancet 2015; 386:2499-506.
209. Bilbo S D, Biedenkapp J C, Der-Avakian A, Watkins L R, Rudy J W, Maier S F. Neonatal infection-induced memory impairment after lipopolysaccharide in adulthood is prevented via caspase-1 inhibition. J Neurosci 2005; 25:8000-9.
210. Bilbo S D, Levkoff L H, Mahoney J H, Watkins L R, Rudy J W, Maier S F. Neonatal infection induces memory impairments following an immune challenge in adulthood. Behav Neurosci 2005; 119:293-301.
211. Noor S, Sanchez J J, Vanderwall A G, Sun M S, Maxwell J R, Davies S, Jantzie L L, Petersen T R, Savage D D, Milligan E D. Prenatal alcohol exposure potentiates chronic neuropathic pain, spinal glial and immune cell activation and alters sciatic nerve and DRG cytokine levels. Brain, behavior, and immunity 2017; 61:80-95.
212. Vanderwall A G, Noor S, Sun M S, Sanchez J E, Yang X O, Jantzie L L, Mellios N, Milligan E D. Effects of spinal non-viral interleukin-10 gene therapy formulated with d-mannose in neuropathic interleukin-10 deficient mice: Behavioral characterization, mRNA and protein analysis in pain relevant tissues. Brain, behavior, and immunity 2018; 69:91-112.
213. Noor S, Milligan E D. Lifelong Impacts of Moderate Prenatal Alcohol Exposure on Neuroimmune Function. Frontiers in immunology 2018; 9:1107.
214. Vargas D L, Nascimbene C, Krishnan C, Zimmerman A W, Pardo C A. Neuroglial activation and neuroinflammation in the brain of patients with autism. Annals of neurology 2005; 57:67-81.
215. Jyonouchi H, Sun S, Le H. Proinflammatory and regulatory cytokine production associated with innate and adaptive immune responses in children with autism spectrum disorders and developmental regression. J Neuroimmunol 2001; 120:170-9.
216. Duggan P J, Edwards A D. Placental inflammation and brain injury in preterm infants. Dev Med Child Neurol Suppl 2001; 86:16-7.
217. Fleiss B, Gressens P. Tertiary mechanisms of brain damage: a new hope for treatment of cerebral palsy? Lancet Neurol 2012; 11:556-66.
218. Dammann O, Leviton A. Maternal intrauterine infection, cytokines, and brain damage in the preterm newborn. Pediatric research 1997; 42:1-8.
219. Dammann O, Leviton A. Intermittent or sustained systemic inflammation and the preterm brain. Pediatric research 2014; 75:376-80.
220. Romero R, Chaemsaithong P, Korzeniewski S J, Tarca A L, Bhatti G, Xu Z, Kusanovic J P, Dong Z, Docheva N, Martinez-Varea A, Yoon B H, Hassan S S, Chaiworapongsa T, Yeo L. Clinical chorioamnionitis at term II: the intra-amniotic inflammatory response. Journal of perinatal medicine 2015.
221. Davies N P, Buggins A G, Snijders R J, Jenkins E, Layton D M, Nicolaides K H. Blood leucocyte count in the human fetus. Arch Dis Child 1992; 67:399-403.
222. Kowalski M L, Wolska A, Grzegorczyk J, Hilt J, Jarzebska M, Drobniewski M, Synder M, Kurowski M. Increased responsiveness to toll-like receptor 4 stimulation in peripheral blood mononuclear cells from patients with recent onset rheumatoid arthritis. Mediators Inflamm 2008; 2008:132732.
223. Robinson S, Franic L A. Chemokine GRO1 and the spatial and temporal regulation of oligodendrocyte precursor proliferation. Developmental neuroscience 2001; 23:338-45.
224. Robinson S, Tani M, Strieter R M, Ransohoff R M, Miller R H. The chemokine growth-regulated oncogene-alpha promotes spinal cord oligodendrocyte precursor proliferation. J Neurosci 1998; 18:10457-63.
225. Duggan P J, Maalouf E F, Watts T L, Sullivan M H, Counsell S J, Allsop J, Al-Nakib L, Rutherford M A, Battin M, Roberts I, Edwards A D. Intrauterine T-cell activation and increased proinflammatory cytokine concentrations in preterm infants with cerebral lesions. Lancet 2001; 358:1699-700.
226. Anthony D, Dempster R, Fearn S, Clements J, Wells G, Perry V H, Walker K. CXC chemokines generate age-related increases in neutrophil-mediated brain inflammation and blood-brain barrier breakdown. Curr Biol 1998; 8:923-6.
227. Fernandez-Lopez D, Faustino J, Daneman R, Zhou L, Lee S Y, Derugin N, Wendland M F, Vexler Z S. Blood-brain barrier permeability is increased after acute adult stroke but not neonatal stroke in the rat. J Neurosci 2012; 32:9588-600.
228. Wang L Y, Tu Y F, Lin Y C, Huang C C. CXCL5 signaling is a shared pathway of neuroinflammation and blood-brain barrier injury contributing to white matter injury in the immature brain. J Neuroinflammation 2016; 13:6.

### Example 2: Extended Combined Neonatal Treatment with Erythropoietin Plus Melatonin Prevents Posthemorrhagic Hydrocephalus of Prematurity in Rats

Posthemorrhagic hydrocephalus of prematurity (PHHP) remains a global challenge. Early preterm infants (<32 weeks gestation), particularly those exposed to chorioamnionitis (CAM), are prone to intraventricular hemorrhage (IVH) and PHHP. We established an age-appropriate, preclinical model of PHHP with progressive macrocephaly and ventriculomegaly to test whether non-surgical neonatal treatment could modulate PHHP. We combined prenatal CAM and postnatal day 1 (P1, equivalent to 30 weeks human gestation) IVH in rats, and administered systemic erythropoietin (EPO) plus melatonin (MLT), or vehicle, from P2 to P10. CAM-IVH rats developed progressive macrocephaly through P21. Macrocephaly was accompanied by ventriculomegaly at P5 (histology), and P21 (ex vivo MRI). CAM-IVH rats showed impaired performance of cliff aversion, a neonatal neurodevelopmental test. Neonatal EPO+MLT treatment prevented macrocephaly and cliff aversion impairment, and significantly reduced ventriculomegaly. EPO+MLT treatment prevented matted or missing ependymal motile cilia observed in vehicle-treated CAM-IVH rats. EPO+MLT treatment also normalized ependymal yes-associated protein (YAP) mRNA levels, and reduced ependymal GFAP-immunolabeling. Vehicle-treated CAM-IVH rats exhibited loss of microstructural integrity on diffusion tensor imaging, which was normalized in EPO+MLT-treated CAM-IVH rats. In summary, combined prenatal systemic inflammation plus early postnatal IVH caused progressive macrocephaly, ventriculomegaly and delayed development of cliff aversion reminiscent of PHHP. Neonatal systemic EPO+MLT treatment prevented multiple hallmarks of PHHP, consistent with a clinically viable, non-surgical treatment strategy.

### Introduction

Posthemorrhagic hydrocephalus of prematurity (PHHP) is a severe form of acquired symptomatic hydrocephalus. PHHP, the combination of ventriculomegaly and elevated intracranial pressure, occurs most commonly in preterm infants who suffer severe intraventricular hemorrhage (IVH) during the first few days of life. Severe IVH is defined as germinal matrix hemorrhage distending a ventricle or involving adjacent parenchyma. Worldwide,˜1.6 million early preterm infants (<32 weeks gestation) are born annually (Blencowe et al., 2012). In countries with advanced medical care, 8-23% of these early preterm infants (˜300,000 infants per year) are afflicted by severe IVH (Alan et al., 2012; Radic et al., 2015; Romero et al., 2015; Poryo et al., 2017). Symptomatic PHHP typically develops weeks after IVH, and represents the worst outcome along a spectrum of severity after severe IVH. The spectrum ranges from ventriculomegaly without signs of elevated intracranial pressure to symptomatic PHHP that requires an invasive intervention. Of surviving early preterm infants with severe IVH, 50-60% will exhibit ventriculomegaly on head ultrasound (HUS) during the newborn admission (Alan et al., 2012; Romero et al., 2015), 20-30% will develop symptomatic hydrocephalus that requires temporary treatment (Alan et al., 2012; Radic et al., 2015), and 16-20% will require permanent intervention (Alan et al., 2012; Radic et al., 2015; Romero et al., 2015). Currently, the only effective treatment for PHHP is surgical intervention (Mazzola et al., 2014), yet most infants and children throughout the world do not have access to safe, timely neurosurgical care (Muir et al., 2016). In the United States, pediatric hydrocephalus treatment consumes $1.4-2 billion dollars annually (2003 dollars) (Simon et al., 2008). The most common neurosurgical intervention for PHHP, insertion of a permanent cerebrospinal fluid (CSF) shunt, brings lifelong dependence on devices prone to malfunction and infection. In a recent multi-center study in the United States, PHHP was the most common reason for initial shunt insertion, and one third of initial infant shunts failed with a mean time to failure of <1 year (Riva-Cambrin et al., 2016). Shunt outcomes are much worse in regions without advanced neurosurgical care (Santos et al., 2017). Other surgical options for infants, such as endoscopic third ventriculostomy with choroid plexus coagulation (ETV-CPC), have not shown superior durability to shunts (Kulkami et al., 2018). The need for safe, effective, economical, non-surgical treatment of PHHP cannot be over-stated.

We propose EMC failure leading to symptomatic hydrocephalus is precipitated by systemic inflammation in combination with a second CNS insult, such as hemorrhage or TBI. Yung et al. (2011) demonstrated that fetal intraventricular lysophosphatidic acid (LPA), a blood-borne inflammatory signaling molecule, precipitates congenital hydrocephalus, and Park subsequently showed that LPA reduces yes-associated protein (YAP), and ependymal maturation (Park et al., 2016). YAP drives the differentiation of radial glial cells toward ependymal cells (Park et al., 2016). Without adequate YAP present, radial glial cells become astrocytes (Huang et al., 2016). We hypothesize that CAM plus early IVH induces symptomatic hydrocephalus that replicates human infant PHHP, and that this occurs in part through alteration of periventricular YAP. Moreover, we propose that combined treatment with EPO and MLT can prevent EMC damage, and thus PHHP. Herein, we report a novel, clinically relevant model of PHHP that combines prenatal injury to induce CNS inflammation plus early postnatal IVH, and prevention of PHHP with neonatal EPO+MLT treatment. This clinically viable, non-surgical intervention prevents acquired hydrocephalus in a preclinical model that exhibits macrocephaly, ventriculomegaly and developmental delay (poor cliff aversion performance), mimicking symptomatic PHHP in human infants.

### Materials and Methods

### Study Design

The primary study objective was to determine if progressive macrocephaly and ventriculomegaly could be reliably induced in neonatal rats using a combination of CAM and IVH, and if combinatorial therapy with EPO+MLT could prevent these two hallmarks of PHHP. For each experiment with a statistical analysis, a power analysis was performed to estimate the required sample size (G*Power 3.1.9.3). We used preliminary and published data to estimate means and standard deviations for each group for the primary outcomes of intra-aural distance, ventriculomegaly, neurodevelopmental assessment, qPCR, and DTI analyses (Farkas et al., 2009; Yung et al., 2011; Baharnoori et al., 2012; Jantzie et al., 2014a,b, 2018; Robinson et al., 2016). Scanning electron microscopy qualitative analysis of EMC was set a priori as two per group. Primary endpoints were set a priori as described in the text, and were daily (P4-P15 and P21) for IAD and neurodevelopment, P5 for ventriculomegaly, P15 for periventricular YAP mRNA, and P21 for remaining assays. No animals were individually excluded except, as noted in the text, those who were removed prior to P21 due to poor health. DTI images from one rat were excluded due to poor image acquisition quality prior to quantification. Rats from both institutions and at least two separate litters were used for all experiments, except rats for SEM were prepared at one institution. Rats from both sexes were randomly assigned to injury (CAM versus sham at E18, IVH versus vehicle at P1) and to treatment group (EPO+MLT or vehicle on P2 for CAM-IVH rats). Each rat pup was coded to ensure all observations and assays and data analyses were performed blinded prior to decoding for interpretation.

### Animals

All procedures followed the National Research Council guidelines, and were performed with the approval of IACUCs at Johns Hopkins University (RA16M369) and the University of New Mexico Health Sciences Center (16-200398-HSC). Timed pregnant Sprague-Dawley dams were purchased (Charles River). Rats had ad libitum food and water, and a standard 12-h light dark cycle. Pups were raised with dams and weaned at P21. Pups were monitored daily for weight and other signs of health. A total of 298 pups were used for this study. Both sexes, and rats from at least two separate litters were used for all experiments. The experimental paradigm is presented in FIG. 7A. ARRIVE guidelines were followed (Kilkenny et al., 2010). An initial cohort of 135 rat pups [43 sham-vehicle (sterile saline), 35 sham-IVH, 27 CAM-vehicle, and 30 CAM-IVH] was used to establish the model of PHHP. A second cohort of 89 rats (31 sham-veh rats, 35 CAM-IVH vehicle-treated rats, and 23 CAM-IVH EPO+MLT-treated rats) was used to test if treatment begun on P2 could prevent the development of PHHP. A third cohort of 74 pups (10 naïve vehicle-treated rats, 20 naïve EPO+MLT-treated rats, 13 vehicle-treated CAM-vehicle rats, 8 vehicle-treated CAM-IVH rats, 5 MLT-treated CAM-IVH rats, 9 EPO-treated CAM-IVH rats, and 9 EPO+MLT-treated CAM-IVH rats) was used for additional control experiments. Whenever possible, each pup was used for multiple assays.

### Chorioamnionitis (CAM) on Embryonic Day 18

To induce CAM, under isoflurane anesthesia pregnant dams underwent laparotomy on embryonic day 18 (E18) with transient uterine artery occlusion for 60 min followed by intra-amniotic injection of lipopolysaccharide (LPS, 0111:B4, Sigma, St. Louis, MO, United States, 4 μl/sac) (Jantzie et al., 2014a, 2015b; Maxwell et al., 2015). The laparotomy was closed and pups were born on E22. Shams underwent anesthesia and laparotomy for 60 min without uterine artery occlusion or LPS injection. 44 dams underwent laparotomy to generate the animals for this study.

### Intraventricular Injection on Postnatal Day 1

For litters with IVH injection, one pup from each litter was randomly chosen as a donor for syngeneic lysed red blood cells (RBCs). Lysed RBCs were chosen because of prior work that suggested lysed RBCs best replicate human IVH (Strahle et al., 2012; Gao et al., 2014). Donor blood was collected, centrifuged at 500×g for 10 min, washed three times with sterile PBS (phosphate-buffered saline), and then lysed with a standardized series of freeze-thaw cycles. On postnatal day 1 (P1), CAM or sham pups were randomly assigned to injection of lysed RBCs (IVH) or vehicle (sterile saline, pH 7.4). The mean hematocrit of P1 lysed RBCs was 37.2±0.9% for shams (n=3) and 36.9±0.6% for CAM rats (n=3). Using hypothermia for sedation, transillumination, and standard landmarks for the lateral ventricle (0.2 mm anterior to the coronal suture and 1 mm lateral of midline), either vehicle or lysed RBCs (20 μl) was injected into each lateral ventricle using a micro-syringe with a 31 gauge needle (Kim et al., 2014; Mukai et al., 2017). Lysed RBCs or vehicle was slowly infused freehand with a percutaneous needle, as previously reported, because neonatal rats at P1 cannot be placed in a stereotactic frame (Mukai et al., 2017). Placement was confirmed visually with transillumination. After 15-20 s the needle was removed, and pups were rewarmed and returned to their dams. Rats were coded and labeled to blind observers for subsequent assessments. Ultrasound (VScan Extend Dual Probe, GE Healthcare) was used to confirm IVH on P2.

### Extended Erythropoietin (EPO) Plus Melatonin (MLT) Treatment

To test if EPO+MLT could prevent the onset of PHHP, on P2 CAM+IVH rats were randomized to receive either EPO+MLT or vehicle from P2 (equivalent to approximately 32 weeks human gestation) to P10 (full term in humans). Six doses of EPO (2500 U/kg/dose i.p.) or vehicle were administered on P2-P5, P7 and P9. Nine doses of MLT (20 mg/kg/dose i.p.) or vehicle were administered on P2-P10. A subset of 36 CAM-IVH pups were randomized to receive vehicle, EPO alone, MLT alone, or EPO+MLT to test impact of treatment on macrocephaly. Shams did not receive EPO+MLT because both EPO and MLT have previously been shown to be safe in these dosing regimens as individual agents (Juul et al., 2008; Mazur et al., 2010; Ohls et al., 2014; Fauchere et al., 2015; Carloni et al., 2017). To determine if EPO+MLT had any unanticipated effect on normal neurodevelopment, a set of naïve pups underwent EPO+MLT treatment. Doses were administered the same time of day to minimize any impact of MLT on the circadian rhythm, although no impact was evident.

### Intra-Aural Distance (IAD)

Observers blinded to injury and treatment group measured 200 rats (cohort 1=87, cohort 2=69, and cohort 3=44). Daily measurements included IAD (Yung et al., 2011) and body weight for dosing calculations.

### Neurodevelopmental Assessment

Neurodevelopmental battery included nine tests (surface righting, negative geotaxis, cliff aversion, forelimb grasp, audio startle, eye opening, ear twitch, forelimb placement, and air righting) (Farkas et al., 2009; Bahamoori et al., 2012). Since no differences with audio startle or eye opening were noted across groups, these two tests were not included for naïve rats. Observers blinded to injury and treatment group tested 146 rats each day from P4 to P15, and then at P21.

### Histology

Animals were anesthetized and perfused with PBS and then 4% paraformaldehyde (PFA), and brains were collected. After immersion in 30% sucrose, coronal frozen sections were cut on a cryostat. Hematoxylin and eosin (HE) staining, immunohistochemistry (IHC) and unbiased stereology were performed, as previously published (Jantzie et al., 2014a). Sections stained with HE were used for ventricular volume. For IHC, briefly, sections were washed and then incubated sequentially with 0.3% hydrogen peroxide and blocking solution containing 10% goat serum in PBS. Primary GFAP antibodies (Dako 1:500, Carpinteria, CA, United States) in blocking solution containing 0.1% Triton were incubated on sections overnight at 4° C. The next morning, sections were rinsed, incubated with species-appropriate biotinylated secondary antibodies, followed by Vectastain (Vector Labs, Burlingame, CA, United States) and 3′3′-diaminobenzidine (DAB). Sections were dehydrated, cleared in xylenes and cover-slipped with Permount (Millipore Sigma, St. Louis, MO, United States). Although dehydration can affect ventricular volume measurements in slide mounted sections, all sections were dehydrated equally, including equivalent times in graded ethanol solutions and xylenes. Appropriate negative controls (no primary antibodies) were run in parallel. Using bright-field illumination, representative images were photographed on a Leica microscope. Observers blinded to injury and treatment status quantified ventricular volumes with Stereologer software (Stereology Resource Center, Tampa, FL, United States) on an upright Leica microscope using optical fractionator methodology under the 40× objective.

### Quantitative PCR

Quantitative PCR was performed as previously published (Jantzie et al., 2014a). Primers for YAP were obtained from the literature (Huang et al., 2016), and verified with BLAST (National Center for Biotechnology Information Nucleotide Basic Local Alignment Search Tool). Briefly, ependyma at P15 were rapidly dissected for RNA isolation (RNeasy Mini kit; Qiagen, Germantown). Following extraction, RNA was transcribed to cDNA, followed by RT-PCR using SYBR green and YAP primers. Experimental replicates were run in triplicate and those CT values varying by more than 0.25 standard deviations were excluded from all analyses. Pooled adult naïve cortex was used to standardize across plates.

### MRI for Ventricular Volume and Microstructural Integrity

Ex vivo MRI T2 and diffusion tensor imaging (DTI) images were acquired at P21 on 33 rats using a Bruker Biospec 7T 70/30 Ultra Shield Refrigerated (USR) nuclear MRI system. Briefly, rats were anesthetized and perfused with PBS, followed by 4% PFA. Brains were post-fixed in 4% PFA for 1 week and embedded in 2% agarose containing 3 mM sodium azide. A T2 multi-slice multi-echo (MSME) sequence was performed with a TR of 3000 ms and TE of 12 ms. FOV was 3 cm×3 cm, with a slice thickness of 1 mm, 12 slices total, and matrix of 256×256, as previously published (Jantzie et al., 2015a; Robinson et al., 2016). Echo-planar diffusion tensor imaging (EP-DTI) images of twelve contiguous coronal 1 mm slices was obtained with a FOV of 3. Ventricular volume was calculated from manually traced regions of interest (ROI) by observers blinded to injury and treatment status. Areas within each slice were calculated, summed across slices and multiplied by slice thickness. DTI was quantified for ROI consistent with previous publications (Jantzie et al., 2015a; Robinson et al., 2016). Because the intraventricular injections were performed bilaterally, for bilateral structures the metrics were acquired on each side and averaged per ROI for each rat.

### Scanning Electron Microscopy (SEM)

Six rats on P21 were perfused with PBS, followed by a mixture of 2.5% glutaraldehyde/2% PFA/0.1 M sodium cacodylate/HCl buffer pH 7.2. Anterior and posterior areas of the lateral or medial walls of the lateral ventricles were micro-dissected and fixed overnight. Anatomically matched tissues were incubated in 1% osmium tetroxide in 0.1 sodium cacodylate/HCl buffer for 4 h, dehydrated in a series of graded ethanol solutions, carbon-coated, then imaged in a Zeiss Sigma 300 field emission scanning electron microscope, consistent with published reports (Sawamoto et al., 2006; Xiong et al., 2014).

### Quantification and Statistical Analysis

In all studies, each rat represents true n. Observers were blinded to injury and treatment status for all data acquisition and initial analyses. For all data sets normal distribution was verified with the Shapiro-Wilk test, with Levene's test to confirm homogeneity of variances. For comparisons of multiple groups that were normal in distribution, two-way ANOVA with Bonferroni's correction was performed (SPSS 24, IBM). For comparison of IADs across groups and ages, a mixed model repeated measures ANOVA with Bonferroni correction was used. For comparison of non-parametric values (categories of ventricular volume), Wilcoxon rank sum test was performed. A Type I error of p<0.05 was considered significant. Mean diffusivity ellipsoids were graphed with MATLAB, R2017b.

### Results

This model incorporates prenatal CNS injury from CAM, early postnatal IVH with syngeneic lysed RBCs, and neurological assessment for 3 weeks through P21 in rodents, a period with some CNS developmental equivalence in humans to early childhood (FIG. 7A). An initial cohort of 135 pups of both sexes was used to characterize a model of PHHP [43 sham-veh (sterile saline) intraventricular injection, 35 sham-IVH, 27 CAM-veh, and 30 CAM-IVH]. Presence of IVH after injection was confirmed by HUS in a subset of pups (FIG. 7B). Early mortality (<48 h) did not differ between injection groups, and was 13% (18/135). No rats from the three control groups (sham-veh, sham-IVH and CAM-veh) became symptomatic from injections, while 4/24 (17%) of CAM-IVH rats that initially survived required euthanasia prior to their targeted endpoint due to progressive macrocephaly accompanied by lethargy and spastic movements. Ventriculomegaly was confirmed on post-mortem examination, and these animals were excluded from further analyses. Surviving rats (113) were collected on P5, P15, and P21.

CAM-IVH Rats Exhibited Progressive Macrocephaly and Ventriculomegaly In human infants, symptomatic PHHP is characterized by progressive macrocephaly with the head circumference crossing percentiles for corrected-age. By P7, CAM-IVH pups developed a dome-shaped cranium (FIG. 7C). To track progressive macrocephaly, intra-aural distance (IAD) was measured daily as a surrogate for human head circumference. Beginning at P5, a significant, progressive increase in IADs was observed in CAM-IVH rats (n=22) through P21, compared to sham-veh (n=33, mixed model repeated measures with Bonferroni's correction, p<0.001), sham-IVH (n=20, p<0.001) and CAM-veh rats (n=19, p<0.001) (FIG. 7D). Both sexes with CAM-IVH exhibited similar patterns of macrocephaly (FIGS. 14A, 14B). Thus, after the combination of perinatal CAM-IVH, neonatal rats displayed progressive macrocephaly through P21, a key component of PHHP.

To quantify early changes in ventricular size, P5 HE-stained coronal sections were analyzed. Ventriculomegaly was observed in CAM-IVH rats (n=9), but not in sham-veh (n=8, two-way ANOVA with Bonferroni correction, p<0.001) or sham-IVH (n=4, p=0.003) rats (FIGS. 7E, 7F). Consistent with our prior work (Jantzie et al., 2014a), CAM-veh rats (n=5) showed moderate ventricular enlargement compared to shams, but smaller than CAM-IVH (all p>0.05). Notably, the sham-IVH and CAM-veh rats with mild ventriculomegaly did not exhibit the progressive macrocephaly that was present only in CAM-IVH rats. These results show prenatal CAM plus postnatal IVH causes early ventriculomegaly.

To clarify whether the ventriculomegaly observed at P5 was transient or sustained, ventricular volumes were analyzed at P21 using ex vivo MRI. Ventricular volumes from CAM-IVH rats (n=8) were significantly larger than sham-veh (n=5), sham-IVH rats (n=4), and CAM-veh rats (n=6, FIG. 7G). Ventricular volume at P21 correlated with macrocephaly (Pearson correlation r=0.338, p=0.012, Table 1). Together, IAD measurements and ventricular volumes from histology and MRI demonstrate that only the combination of prenatal CAM plus early postnatal IVH leads to progressive macrocephaly with persistent ventriculomegaly, characteristic of PHHP.

TABLE 1

| | | Cliff aversion | | | Ventricular volume | | | | | |
|---|---|---|---|---|---|---|---|---|---|---|
| Parameter | | Pearson r | p-value | | Pearson r | | | p-value | | |
| Macrocephaly (P21 IAD) | | 0.281 | p = | 0.040 | 0.338 | | | p = 0.012 | | |
| Cliff aversion | | [symbol] | | | 0.461 | | | p < 0.001 | | |
| | | Macrocephaly P21 IAD | | | Cliff aversion | | | Ventricular volume | | |
| DTI scalars | | Pearson r | p-value | | Pearson r | p-value | | Pearson r | p-value | |
| White matter | FA | −0.585 | p = | 0.001 | −0.447 | p = | 0.012 | −0. [symbol] | p = | 0.040 |
| Corpus callosum | FA | −0.427 | p = | 0.015 | −0.361 | p = | 0.006 | −0. [symbol] | p = | 0.070 |
| Hippocampus | FA | −0. [symbol] | p = | 0.001 | −0.247 | p = | 0.181 | −0.099 | p = | 0. [symbol] |
| Striatum | FA | −0.318 | p = | 0.076 | −0.444 | p = | 0.012 | −0.205 | p = | 0. [symbol] |
| Thalamus | FA | −0.201 | p = | 0.27 [symbol] | −0.320 | p = | 0.079 | −0.210 | p = | 0.249 |
| White matter | MD | 0.422 | p = | 0.016 | 0.320 | p = | 0.079 | 0.291 | p = | 0.106 |
| Corpus callosum | MD | 0.467 | p = | 0.007 | 0. [symbol] | p = | 0.034 | 0. [symbol] | p = | 0.030 |
| White matter | RD | 0.4 [symbol] | p = | 0.018 | 0. [symbol] | p = | 0.033 | 0. [symbol] | p < | 0.000 |
| Corpus callosum | RD | 0.552 | p = | 0.001 | 0. [symbol] | p = | 0.006 | 0.445 | p = | 0.011 |

Bolded values indicate statistical significance. [symbol]
indicates data missing or illegible when filed

### Cumulative Insults Precipitated Developmental Delay

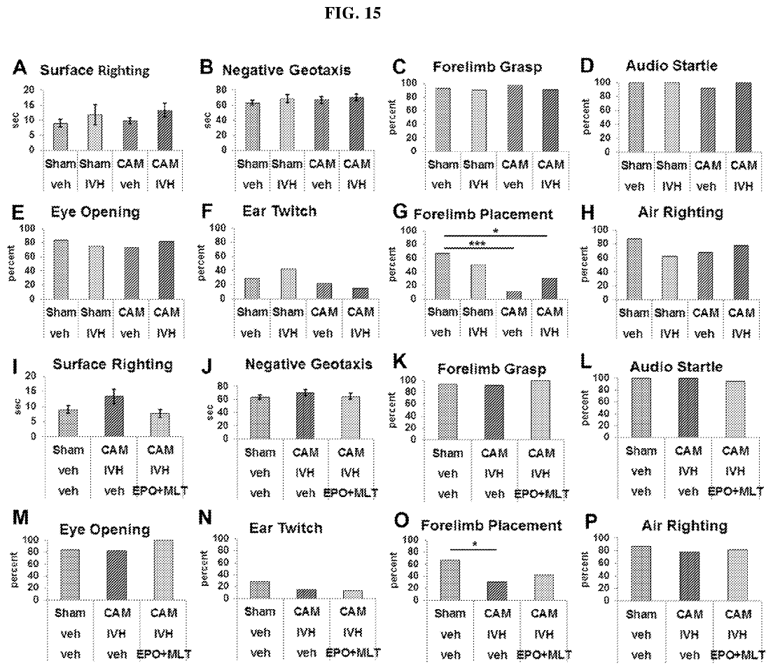

To assess neonatal neurodevelopment, a battery of age-appropriate, validated, functional tests (Farkas et al., 2009; Baharnoori et al., 2012) was administered. Most responses, including somatic reflexes such as eye opening and startle response, did not vary amongst the four groups (FIG. 15). The forelimb placement reflex, the placement of the forelimb on a shelf in response to stroking the dorsum of the forelimb while the rat is suspended near the shelf, was worse in CAM-veh and CAM-IVH rats compared to sham-veh rats (FIG. 15G). Cliff aversion, the time it takes a pup placed with its head and forelimbs over a ledge to return to a safe position, was longer in injured rats, consistent with early developmental delay. Specifically, CAM-IVH rats (n=24) exhibited longer cliff aversion times compared to sham-veh rats (n=24, p=0.002), and sham-IVH rats (n=18, p<0.001, FIG. 7H). The CAM-veh rats (n=31) also showed impairment with cliff aversion, but did not differ significantly from sham-veh or CAM-IVH rats. The pattern of poor cliff aversion performance by CAM-IVH rats was more pronounced in male rats than female rats (FIGS. 14E, 14F). Notably, cliff aversion performance correlated with macrocephaly (r=0.281, p=0.040), and more robustly with ventriculomegaly (r=0.461, p<0.001, Table 1), suggesting that in this model early functional outcomes are associated with macrocephaly and ventriculomegaly.

Correlation of Phenotype with Early CNS Insults

To estimate the relative contribution of early CNS insults to phenotypic parameters important for PHHP, correlations were tested between individual findings and types of CNS insults. Macrocephaly (P21 IAD) correlated only with the combination of CAM-IVH (r=0.420, p=0.010), and not with CAM or IVH in isolation (Table 2). Similarly, ventriculomegaly correlated only with CAM-IVH (r=0.765, p=0.002), and not with CAM or IVH alone. Poor cliff aversion performance also correlated with CAM-IVH (r=0.467, p=0.004, Table 2). Overall, only the combined CAM-IVH correlated robustly with macrocephaly, ventriculomegaly, and poor functional performance that are characteristic of PHHP.

TABLE 2

| | | [symbol] | [symbol] |
|---|---|---|---|
| | | plus IVH | plus IVH |
| [symbol] | IVH | (vehicle) [symbol] | (EPO + MLT) [symbol] |

| Clinical parameter | Pearson r | p-value | Pearson r | p-value | Pearson r | p-value | Pearsonr | p-value |
|---|---|---|---|---|---|---|---|---|
| [symbol] (P21 IAM) | [symbol] | p = 0.574 | [symbol] | p = [symbol] | 0.420 | p = 0.010 | −0.599 | p < 0.001 |
| Cliff aversion | 0.323 | p = [symbol] | [symbol] | p = [symbol] | 0.467 | p = 0.004 | −0.367 | p = 0.042 |
| Ventricular volume | [symbol] | p = 0.100 | [symbol] | p = [symbol] | 0.765 | p = 0.002 | −0.341 | p = 0.056 |

| | | | | | [symbol] | | [symbol] | |
|---|---|---|---|---|---|---|---|---|
| | | | | | plus IVH | | plus IVH | |
| | [symbol] | | IVH | | (vehicle) [symbol] | | (EPO + MLT) [symbol] | |
| DTI scaler | Pearson r | p-value | Pearson r | p-value | Pearson r | p-value | Pearson r | p-value |
| [symbol] | [symbol] | p = [symbol] | [symbol] | p = 0.071 | [symbol] | p = 0.002 | [symbol] | p < 0.001 |
| [symbol] | [symbol] | p = [symbol] | [symbol] | p = [symbol] | [symbol] | p = 0.001 | [symbol] | p < 0.001 |
| Hippocampus FA | [symbol] | p = [symbol] | [symbol] | p = [symbol] | [symbol] | p = 0.004 | [symbol] | p = 0.001 |
| [symbol] FA | [symbol] | p = [symbol] | [symbol] | p = [symbol] | [symbol] | p = 0.018 | [symbol] | p < 0.001 |
| [symbol] FA | [symbol] | p = [symbol] | [symbol] | p = [symbol] | [symbol] | p = 0.019 | [symbol] | p = 0.012 |
| White matter MD | [symbol] | p = [symbol] | [symbol] | p = [symbol] | [symbol] | p = 0.001 | [symbol] | p = [symbol] |
| [symbol] MD | [symbol] | p = [symbol] | [symbol] | p = [symbol] | [symbol] | p < 0.001 | [symbol] | p = 0.003 |
| White matter RD | [symbol] | p = [symbol] | [symbol] | p = [symbol] | [symbol] | p < 0.001 | [symbol] | p = 0.173 |
| Corpus callosum RD | [symbol] | p = [symbol] | [symbol] | p = [symbol] | [symbol] | p < 0.001 | [symbol] | p < 0.001 |

Bolded values indicate

[symbol]

[symbol]

[symbol]

EPO + MLT [symbol] [symbol]
indicates data missing or illegible when filed

### EPO+ML T Prevented Macrocephaly and Neurodevelopmental Delay in PHHP-Like Rats

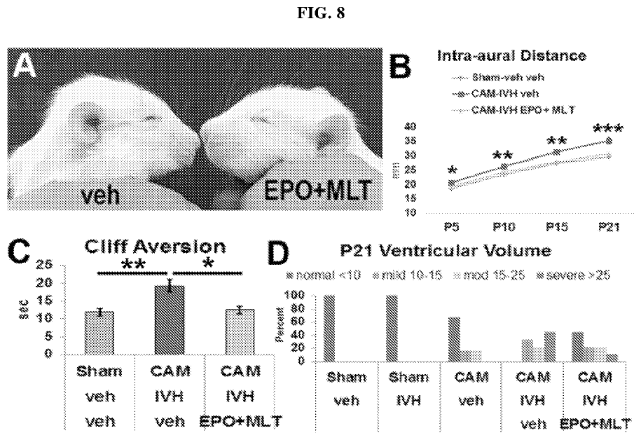

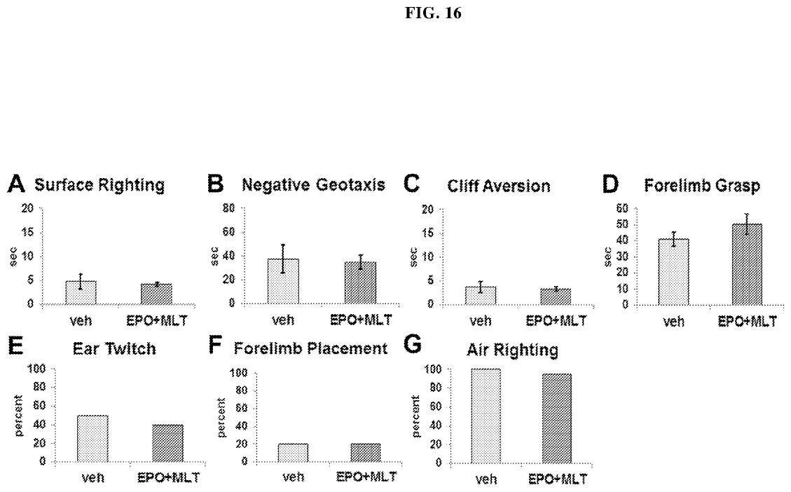

To determine if the progressive macrocephaly and poor cliff aversion performance observed in CAM-IVH rats was preventable, we assessed the efficacy of combined EPO+MLT treatment in the neonatal period in a separate cohort of rats (n=88). Specifically, on P2, CAM-IVH rats were randomized to an extended regimen of EPO+MLT or vehicle from P2 to P10. To determine whether the combination of EPO+ML T had any unanticipated impact on neurodevelopment, naïve rats were randomized to the same EPO+MLT regimen (n=20), or vehicle (n=10). No differences in neurodevelopment were detected (FIG. 16), consistent with prior studies showing no detrimental effects of EPO or MLT in shams (Mazur et al., 2010; Carloni et al., 2014, 2016). Thus, shams received only vehicle for the remainder of the studies. While vehicle-treated CAM-IVH rats exhibited a domedhead, EPO+MLT-treated CAM-IVH rats did not (FIG. 8A). At P5, the IADs of the EPO+ML T-treated CAM-IVH rats (n=15) were significantly lower than IADs of the vehicle-treated CAM-IVH rats (n=28, mixed model repeated measures with Bonferroni correction, p=0.017, FIG. 8B). By P21, the IADs of the EPO+MLT-treated Mn similar to shams (n=28), and were markedly less than the vehicle-treated CAM-IVH rats (p<0.001). By contrast, when EPO alone (n=9), or MLT alone (n=5) were administered to CAM-IVH rats, the IADs were not significantly different compared littermates that received vehicle (n=8, FIG. 14I). The EPO+MLT treatment prevented progressive macrocephaly in both male and female rats (FIGS. 14C, 14D). In addition to preventing macrocephaly, neonatal EPO+MLT treatment also mitigated poor performance on cliff aversion after CAM-IVH. Compared to vehicle-treated CAM-IVH rats (n=24), EPO+MLT-treated CAM-IVH rats (n=17) exhibited shorter cliff aversion times (p=0.026), and performed similar to shams (n=24, FIG. 8C). None of the other reflexes were significantly impacted by EPO+MLT, although a trend for surface righting was apparent (FIG. 15). While CAM-IVH rats of both sexes showed a similar pattern of improved cliff aversion performance with neonatal EPO+MLT treatment, the differences were more prominent in males (FIGS. 14G, 14H). Thus, neonatal EPO+MLT treatment prevented progressive macrocephaly and early signs of developmental delay exhibited by vehicle-treated CAM-IVH rats.

### EPO+MLT Treatment Improved Ventriculomegaly in PHHP-Like Rats

To determine if EPO+MLT treatment impacted ventriculomegaly, we evaluated ventricular volume at P21. Ventricular volume from ex vivo MRI was classified a priori as normal (<10 mm³), mild (10-<15 mm³), moderate (15-25 mm³) or severe (>25 mm³) ventriculomegaly (FIG. 8D). All of the sham-veh (5/5) and sham-IVH (4/4) rats had normal ventricular volume. Two-thirds (4/6) of CAM-veh rats exhibited a normal volume, while one-third had mild or moderate ventriculomegaly without macrocephaly, consistent with posthemorrhagic ventricular dilation observed in some toddlers born preterm with CNS injury (Pappas et al., 2018). All (9/9) of the vehicle-treated CAM-IVH rats showed ventriculomegaly (33% mild, 22% moderate, and 44% severe). By contrast, 44% (4/9) of EPO+MLT-treated rats had a normal ventricular volume and only 110% had severe ventriculomegaly, a significant reduction in the proportion with ventriculomegaly compared to vehicle-treated CAM-IVH rats (Wilcoxon rank sum test, p=0.005). The reduction of ventriculomegaly combined with the lack of macrocephaly suggests that PHHP occurs along a spectrum of severity that can be modulated with a timely neuroreparative intervention.

Microstructural Abnormalities Improved with EPO+MLT Treatment

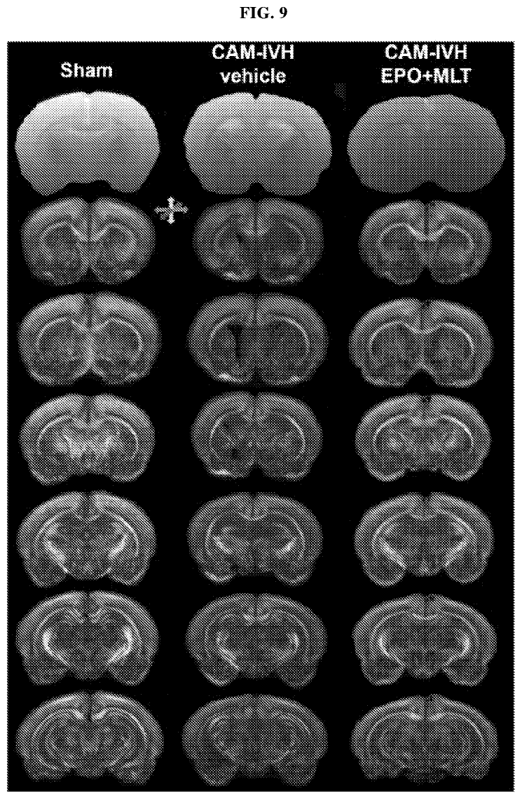

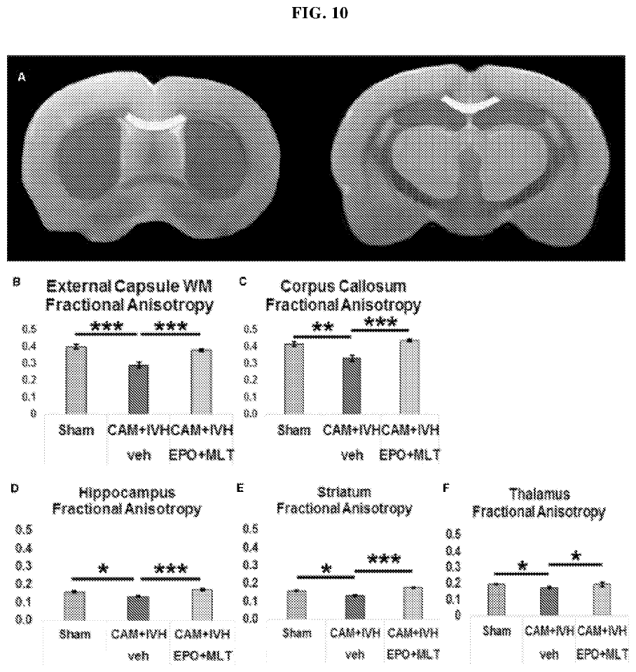

Due to the challenges in obtaining reliable functional outcomes in neonates, DTI has emerged as an imaging biomarker for perinatal brain injury (Vollmer et al., 2017; Hollund et al., 2018; Olsen et al., 2018). In particular, congenital hydrocephalus in children is associated with widespread DTI microstructural abnormalities (Mangano et al., 2016; Zhao et al., 2016). To supplement the functional outcome quantified with the behavior battery, we assessed regional variation in white and gray matter microstructure (FIG. 9 and FIG. 10A). Vehicle-treated CAM-IVH rats (n=8) had markedly lower external capsule white matter (ECWM) fractional anisotropy (FA) at P21 compared to shams (n=5, two-way ANOVA with Bonferroni's correction p<0.001), that improved with EPO+MLT (n=9, p=0.001, FIG. 10B) A similar pattern with FA was observed in the central corpus callosum (shams versus vehicle-treated CAM-IVH, p=0.002; vehicle-treated versus EPO+MLT-treated CAM-IVH, p<0.001, FIG. 10C). Likewise, gray matter FA was lower in vehicle-treated CAM-IVH rats compared to shams in the hippocampus (p=0.022, FIG. 10D), striatum (p=0.028, FIG. 10E) and thalamus (p=0.030, FIG. 10F). Significantly, neonatal EPO+MLT treatment in CAM-IVH rats prevented loss of gray matter microstructural integrity in hippocampus (p<0.001), striatum (p<0.001), and thalamus (p=0.018). Thus, while CAM-IVH causes extensive loss of microstructural integrity by P21, neonatal EPO+MLT treatment prevents this widespread damage.

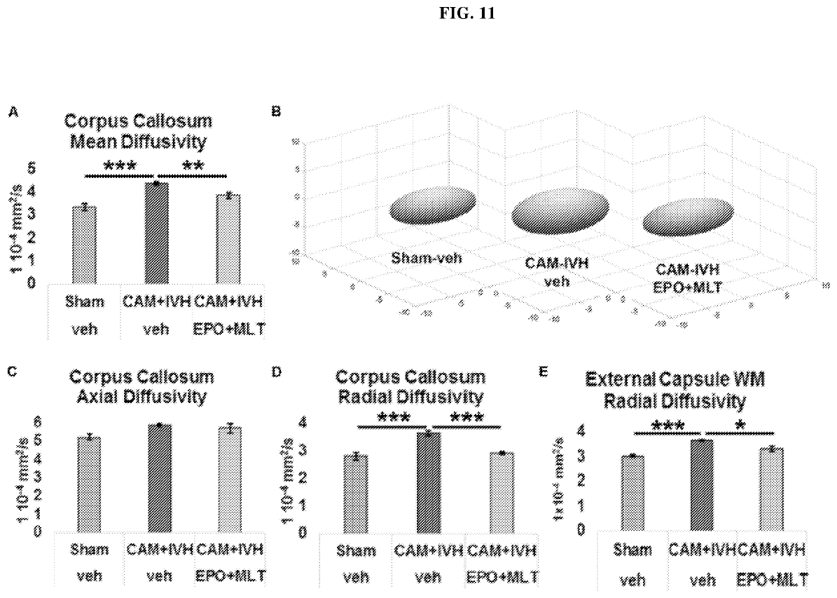

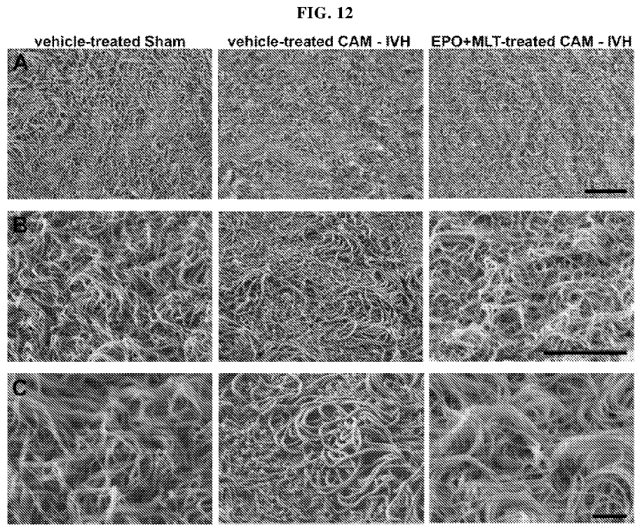

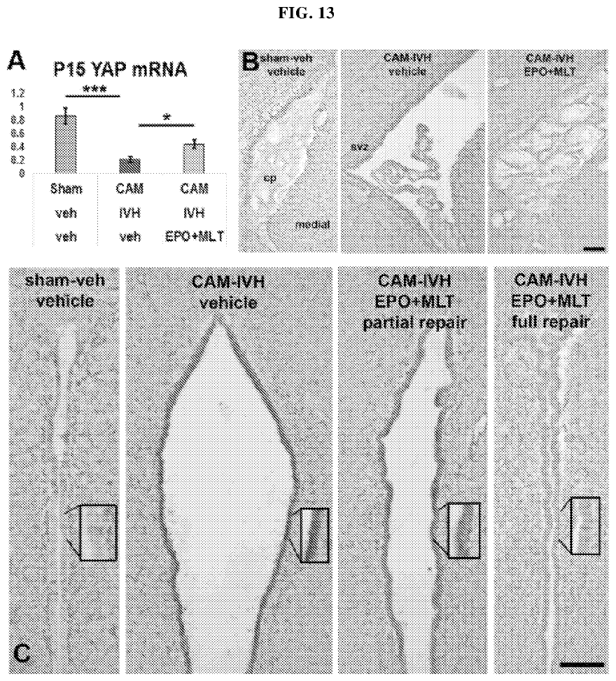

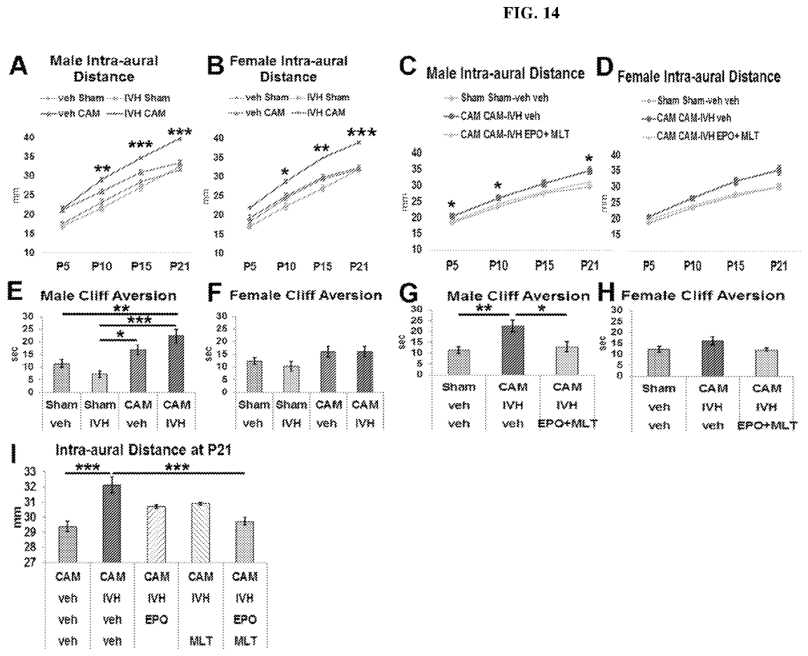

To more specifically examine microstructural integrity in the corpus callosum and ECWM, we assessed directional diffusion. Mean diffusivity (MD) was elevated in the corpus callosum of vehicle-treated CAM-IVH rats compared to shams (p=0.001) and EPO+MLT-treated CAM-IVH rats (p=0.008, FIGS. 11A, 11BB). Axial diffusivity was not altered in the corpus callosum of CAM-IVH rats (FIG. 11C). By contrast, radial diffusivity (RD), a potential imaging biomarker of myelin injury, was also elevated in the corpus callosum of vehicle-treated CAM-IVH rats, compared to shams and EPO+MLT-treated CAM-IVH rats (both p<0.001, FIG. 11D). Similarly, ECWM RD was elevated in vehicle-treated CAM-IVH rats, compared to shams (p=0.001) and EPO+MLT-treated CAM-IVH rats (p=0.027, FIG. 11E). Together, these findings show that the corpus callosum and ECWM are particularly vulnerable to CAM-IVH, and that early EPO+MLT treatment can prevent microstructural abnormalities caused by prenatal CAM plus IVH.

We tested correlations of DTI scalars with macrocephaly, impaired cliff aversion performance, and ventriculomegaly (Table 1). Macrocephaly (P21 IAD) correlated robustly with FA in the ECWM (p=0.001), corpus callosum (p=0.015) and hippocampus (p=0.001). Similarly, poor cliff aversion performance correlated strongly with FA in the ECWM (p=0.012), corpus callosum (p=0.006), and striatum (p=0.012), while ventriculomegaly correlated with corpus callosum FA (p=0.04). For mean diffusivity, all three phenotypic metrics (macrocephaly, poor cliff aversion performance, and ventriculomegaly) showed strong correlation with corpus callosum MD (all p<0.03, Table 1), while only macrocephaly was associated with altered ECWM MD (p=0.016). Moreover, for radial diffusivity, all three phenotypic metrics showed strong correlation with both corpus callosum and ECWM RD (all p<0.035, Table 1). Together, these finding show DTI scalars, particularly RD of white matter, are indicative of phenotypic parameters that most closely resemble PHHP, and suggest that metrics of directional diffusion may serve as biomarkers of PHHP severity and recovery.

Next, to test the impact of different components of CNS injury on DTI scalars, we examined the correlations between CAM, IVH, and CAM-IVH compared to shams. Only thalamic FA correlated with CAM alone (p=0.035), and only striatal FA showed a trend with IVH alone (p=0.052, Table 2). By contrast, FA from both white and gray matter correlated with CAM-IVH (all p<0.02). Moreover, for both ECWM and corpus callosum, both MD and RD showed very robust correlations with CAM-IVH (all p<0.001). Finally, the correlations between DTI scalars for vehicle-treated compared to EPO+MLT-treated CAM-IVH rats were analyzed. These also showed strong correlations with DTI scalars when compared with vehicle-treated CAM-IVH rats (Table 2). Thus, in addition to DTI correlating with phenotypic parameters, DTI scalars may hold promise as imaging biomarkers for specific CNS insults associated with PHHP, and neurorepair.

### EPO+MLT Treatment Ameliorated Morphological Damage to Ependymal Motile Cilia

To examine EMC morphology, scanning electron microscopy (SEM) was performed on anatomically matched micro-dissected portions of the lateral ventricular wall on P21. Cilia from sham rats appeared in organized tufts (FIG. 12). By contrast, a subset of cilia from vehicle-treated CAM-IVH rats were missing, while the remaining cilia were matted, flattened, and lacked the tuft-like appearance found in shams. Importantly, EPO+MLT treatment prevented the damage and restored the tuft-like appearance of EMC in CAM-IVH rats. Thus, these results demonstrate neonatal EPO+MLT treatment can modulate structural damage to EMC after CAM-IVH.

EPO+MLT Prevented Low Yes-Associated Protein (YAP) mRNA Levels at P15

Next, we tested whether EPO+MLT treatment impacted YAP mRNA transcription levels in micro-dissected ependyma at P15. Reduction of YAP mRNA levels occurred in vehicle-treated CAM-IVH rats (n=4), compared to shams (n=7, p<0.001, FIG. 7A). Neonatal EPO+MLT treatment following CAM-IVH (n=5) mitigated loss of YAP mRNA, compared to vehicle treated CAM-IVH (two-way ANOVA with Bonferroni correction, p=0.036). These results provide initial support for our hypothesis that CAM-IVH reduces YAP transcription, and that EPO+MLT treatment prevents loss of YAP mRNA.

### EPO+MLT Treatment Reduced Excess Ependymal GFAP Expression

To determine whether CAM-IVH substantially altered periventricular gliosis, P21 coronal sections were immunolabeled with GFAP (glial fibrillary acidic protein) antibodies. In the lateral ventricle, GFAP-immunolabeling in vehicle-treated CAM-IVH rats differed from shams, and demonstrated changes in both the ependyma and choroid plexus (FIG. 7B). After EPO+MLT treatment, the ependyma showed less GFAP+ reactivity, and the choroid plexus resembled the choroid plexus of shams. Ependymal changes were more dramatic in the third ventricle (FIG. 7C), consistent with a diffuse ependymal reaction after CAM-IVH. Sham rats showed no enlargement of the third ventricle, and minimal GFAP+ cells in the ependymal lining (FIG. 7C). By contrast, ependyma in vehicle-treated CAM-IVH rats with severe ventriculomegaly exhibited dense GFAP+ expression, consistent with ventricular disruption observed in human infants with IVH (McAllister et al., 2017). GFAP-immunolabeling was moderate in EPO+MLT-treated CAM-IVH rats with residual mild ventriculomegaly, while EPO+MLT-treated rats with normal ventricular size had minimal GFAP-immunolabeling, similar to shams. Notably, in this age-appropriate CAM-IVH model, the degree of GFAP expression appeared to vary inversely with ventriculomegaly.

### DISCUSSION

We developed a clinically relevant model of PHHP that exhibits essential components of PHHP, progressive macrocephaly with ventriculomegaly, and used this model to test whether extended EPO+MLT treatment can prevent the development of PHHP. We found that EPO+MLT prevented progressive macrocephaly and impaired cliff aversion performance, and reduced ventriculomegaly. Neonatal EPO+MLT treatment also normalized damage from CAM-IVH to microstructural integrity of white and gray matter, ultrastructural injury to EMC, periventricular YAP levels, and ependymal GFAP-immunolabeling. These data indicate that the combination of inflammation from prenatal CAM plus early postnatal IVH damages EMC, likely limiting propulsion of CSF and leading to symptomatic PHHP. The well-characterized prenatal injury model of CAM used here causes pathological and functional changes that mimic consequences of CNS injury from extremely preterm birth (Jantzie et al., 2014a, 2018; Maxwell et al., 2015). In juvenile rats, prenatal CAM produces a gait deficit reminiscent of the spastic gait of cerebral palsy (Jantzie et al., 2014a), and impaired executive function, social interaction and hyperactivity (Jantzie et al., 2018). Importantly, because of the staggered timing of CNS development and birth in rats compared to humans [birth in Sprague-Dawley rats is equivalent approximately to 30 weeks gestation in humans, and P7-P10 in the rat is equivalent to a term human (Semple et al., 2013; Jantzie and Robinson, 2015)], newborn rats mimic early preterm neonates and allow age-appropriate testing of interventions. This novel model of PHHP mimics the prenatal impact of CAM and intrauterine inflammation on CNS maturation, plus replicates the timing of IVH in early preterm infants by injecting lysed RBCs on P1. Pups exposed to prenatal CAM-IVH developed persistent ventriculomegaly with progressive macrocephaly, two hallmarks of PHHP, accompanied by histological, functional and imaging findings consistent with those observed in preterm infants with PHHP. EMC in CAM-IVH rats exhibited damage, similar to damaged EMC observed after intracerebral hematoma and TBI in the mature CNS (Xiong et al., 2014; Chen et al., 2015). The combination of macrocephaly with ventriculomegaly reported here is also similar to the morphology of models of symptomatic hydrocephalus resulting from genetic mutations that impede EMC development and/or function (Banizs et al., 2005; Tissir et al., 2010; Yung et al., 2011; Peng et al., 2013; Vidovic et al., 2015; Park et al., 2016; Muniz-Talavera and Schmidt, 2017; Nunez-Olle et al., 2017; Takagishi et al., 2017; Abdelhamed et al., 2018; Xu et al., 2018). Together, the unique combination of CAM plus IVH, along with the age-appropriate timing of both insults, increases the clinical relevance of this novel model.

We used this PHHP model to test whether systemic, extended high-dose neonatal treatment with EPO+MLT could promote recovery and prevent hydrocephalus. PHHP results from a complex pattern of cumulative insults over the perinatal period, and extended dosing with EPO+MLT likely normalizes the microenvironment and enhances recovery over a sustained critical neurodevelopmental window. Preterm infants have different clinical risks of CNS damage, arising from varying patterns of IVH distribution and severity, plus other forms of neonatal injury such as periventricular leukomalacia and sepsis, and the genetic predisposition that impacts the response to these cumulative insults. The extended dosing period and combinatorial therapies likely addresses the varying severity, timing and individual predisposition to damage better than using a single agent over the developmental window. Also, CAM and IVH both affect multiple cell types in widespread CNS regions, a complex amalgam that has been identified in other forms of CNS injury from preterm birth (Volpe, 2009). This diffuse, complex, cumulative pattern of injury sustained over several weeks by preterm infants differs substantially from a regionally and temporally specific injury such as a single occlusive middle cerebral artery stroke. EPO and MLT also have numerous overlapping complementary mechanisms of action on neural cells that result in repair (Brines et al., 2000; Carloni et al., 2008). Receptors for both EPO and MLT are present on neural cells (Mazur et al., 2010; Bahna and Niles, 2017; Ng et al., 2017; Osier et al., 2017; Tsai et al., 2017), and both EPO and MLT enhance the genesis, survival and maturation of multiple neural cell types (Iwai et al., 2010; Mazur et al., 2010; Jantzie et al., 2013; Li et al., 2017; Zhang et al., 2018). EPO and MLT reduce neuroinflammation and oxidative stress (Carloni et al., 2016; Ramirez-Jirano et al., 2016; Dominguez Rubio et al., 2017; McDougall et al., 2017; Wang et al., 2017; Wei et al., 2017; Zhou et al., 2017). They also restore the microenvironment by limiting excess calpain activity that promotes cell death and destroys molecules essential for neurodevelopment (Samantaray et al., 2008; Jantzie et al., 2014b, 2016; Robinson et al., 2016). EPO and MLT reduce endoplasmic reticulum stress and mitochondrial dysfunction that may propagate chronic damage and precipitate early neurodegeneration (Hong et al., 2012; Das et al., 2013; Carloni et al., 2014; Fernandez et al., 2015; Hadj Ayed Tka et al., 2015; Zhao et al., 2015; Hu et al., 2016; Hardeland, 2017; Mendivil-Perez et al., 2017; Xue et al., 2017). Additionally, EPO+MLT treatment likely suppresses LPA, increases YAP, and enhances ependymal renewal, which when combined with white matter repair, provides a unique advantage to stimulate recovery from the cascade of insults the cumulatively lead to PHHP. Further work to delineate the specific mechanisms of how EPO and MLT act together to optimize EMC maturation following CAM-IVH are underway.

Systemic inflammation, particularly from maternal causes, is associated with a higher risk of IVH (Moscuzza et al., 2011; Salas et al., 2013; Arayici et al., 2014; Shankaran et al., 2014; Lee et al., 2016; Lu et al., 2016; Stark et al., 2016; Chevallier et al., 2017; Edwards et al., 2018). The Gram-negative endotoxin LPS used here binds TLR4 (toll-like receptor 4), a receptor implicated in neonatal parenchymal microhemorrhages (Carusillo Theriault et al., 2017). Others have also shown that a TLR4 antagonist limits damage from intracerebral hemorrhage (Wang et al., 2013; Kwon et al., 2015). Systemic inflammation mediated via TLR4 increases choroid plexus CSF secretion (Karimy et al., 2017), and likely contributes to ventriculomegaly in hydrocephalus. We found that EPO+MLT treatment not only limited ependymal GFAP-expression, but also prevented altered morphology of the choroid plexus. Thus, there are multiple potential mechanisms to explain why this combinatorial therapy limits the development of PHHP.

We used this novel PHHP model to test the potential reversibility of ependymal injury. Based on prior work (Yung et al., 2011; Huang et al., 2016; Park et al., 2016), we reasoned that one potential explanation connecting systemic inflammation to the propensity to develop PHHP after IVH is that systemic inflammation from LPS impacts ependymal cells, and potentially neural cell progenitor differentiation, through alterations in periventricular YAP mRNA levels. Here we report that CAM-IVH reduces YAP transcription in micro-dissected ependyma, and that neonatal EPO+MLT prevents loss of YAP mRNA. These data are consistent with a role for YAP in the mitigation of EMC damage, as observed with SEM in this report.

Studies using DTI in children with hydrocephalus have shown alterations in white matter microstructural integrity (Mangano et al., 2016; Zhao et al., 2016), similar to what we have shown here in toddler-equivalent P21 vehicle-treated CAM-IVH rats. In preclinical studies, extended neonatal EPO treatment mitigated widespread DTI abnormalities in white and gray matter (Robinson et al., 2016, 2017). Here, we showed that sustained neonatal treatment with EPO+MLT normalizes subacute DTI changes at 3 weeks after CAM-IVH. To our knowledge, this is the first report of the prevention of DTI microstructural damage in a preclinical model of PHHP. Aojula et al. (2016) used the transforming growth factors-β antagonist decorin to prevent DTI changes at 2 weeks in a kaolin model of hydrocephalus. Similarly, neonatal kaolin resulted in DTI abnormalities at 5 and 10 days (Yuan et al., 2010). The DTI analyses here demonstrated that both white matter and gray matter, and particularly the corpus callosum, are sensitive to CAM-IVH. While a component of white matter vulnerability may be related to direct structural injury from ventriculomegaly, it is likely that myelination and EMC function are precise, highly regulated, relatively vulnerable processes with demanding energy expenditures and environmental requirements. We reasoned that the precise regulation of EMC maturation and function, as well as recovery of more widespread injury to white and gray matter after CAM-IVH, required a potent, multipronged intervention. This led us to test a neonatal, sustained, high-dose, neuroprotective, clinically relevant dosing regimen of EPO+MLT.

While clinical studies have shown that isolated neonatal symptomatic hydrocephalus does not cause chronic neurological deficits (Radic et al., 2015), children with PHHP often suffer from additional neurological co-morbidities such as cerebral palsy, behavioral abnormalities and impaired cognition. Prenatal inflammation plus postnatal inflammation increases the risk of white matter injury and spastic cerebral palsy (Yanni et al., 2017), and many preterm infants with IVH suffer from neonatal inflammation that likely contributes to their poor functional outcomes. A sustained EPO+MLT regimen, similar to the one used here, prevents chronic motor, social, behavioral and cognitive deficits in adult rats following prenatal CAM (Jantzie et al., 2018). In this study EPO+MLT treatment prevented poor performance on cliff aversion, however, tests of neonatal function in rodents are inherently limited. Using a strategy similar to EPO+MLT to induce multi-faceted repair, intravenous or intraventricular injection of umbilical-cord derived mesenchymal stromal cells on P6 improved early functional outcomes and reduced ventriculomegaly and GFAP-expression in rats with IVH on P4 or P5 (Ahn et al., 2013; Mukai et al., 2017). Repetitive dosing with EPO+MLT has the distinct advantages of providing sustained stabilization of the microenvironment over extended perinatal course, known safety profile, ease of administration, and cost. Early intervention for ventriculomegaly from IVH may improve long term cognition in very selected populations (Leijser et al., 2018), yet preterm infants with two or more surgeries as neonates have lower cognitive scores (Gano et al., 2015), and shunt surgery at a young age increases the risk of shunt malfunction (Riva-Cambrin et al., 2016). While the timing of interventions for sick preterm infants with PHHP is a complex and evolving controversy, non-surgical intervention such as neonatal EPO+MLT could potentially minimize the need for more invasive procedures and their associated risks.

The pathogenesis of PHHP is likely similar to other types of acquired hydrocephalus from TBI, adult IVH, subarachnoid hemorrhage, or meningitis. The association between CNS inflammation and hydrocephalus is well known (Del Bigio and Di Curzio, 2016). More recently the association with systemic inflammation has been found. Sepsis increases the risk of developing hydrocephalus in infants and young children with TBI (Rumalla et al., 2018). Similarly, Wessell et al. (2018) showed that sustained systemic inflammatory response syndrome (SIRS) increases the risk of developing shunt-dependent hydrocephalus after aneurysmal subarachnoid hemorrhage. Adults with severe TBI are more likely to need surgical treatment for hydrocephalus in the presence of ventriculitis/meningitis (Bauer et al., 2011; Hu et al., 2018). Interestingly, after adult stroke in a preclinical model, increased GFAP expression was found in the ependymal lining, and EMC were affected, but hydrocephalus did not develop (Young et al., 2013). Numerous differences in the manifestations of injury and potential for repair exist between the developing and mature CNS. Current preclinical experiments are underway to determine whether findings from this study are relevant to acquired hydrocephalus in adults.

This study has several limitations. Many important mechanistic questions are beyond the scope of this initial report. Additional experiments are needed to clarify the limits on the timing and extent of renewal of ependymal cells and EMC, and thus the duration of the optimal dosing regimen. While the results presented here support our hypothesis that EMC damage in the neonatal period can be modulated significantly by endogenous neuroprotective agents, the situation in human neonates with PHHP is likely more complex. More specifically targeted therapies may add benefit, particularly in vulnerable patients due to their genetic or epigenetic risk factors. Still, the findings presented here suggest that neonatal EPO+MLT may offer a safe, effective, cost-sensitive treatment for at least a subset of infants with PHHP. Additional studies are underway to test cognitive outcomes in young adult rats after CAM-IVH. This is exceedingly important as children with ventricular dilatation after IVH of prematurity have cognitive deficits (Holwerda et al., 2016), and those who require surgical intervention are at even higher risk for poor outcomes (Adams-Chapman et al., 2008; Holwerda et al., 2016). Elevated LPA levels from systemic inflammation may reduce YAP transcription and its specification of radial glial cells to differentiate into ependymal cells, however, investigation of this hypothesis requires additional studies to clarify the timing of damage, and therapeutic window for repair. Our initial findings suggest that ventriculomegaly in this model is associated with diffuse ependymal GFAP+ reaction, however, any potential causal relationship is currently unknown. Experiments to evaluate the long term impact of CAM-IVH and the association with macrocephaly and ventriculomegaly are underway. Moreover, while we demonstrated that both sexes develop progressive macrocephaly, the study was not sufficiently powered to determine how well each sex responds to EPO+MLT treatment. Despite these and other limitations, this novel model of PHHP and our initial findings using the clinically viable EPO+MLT treatment to prevent macrocephaly with ventriculomegaly, poor cliff aversion performance and associated microstructural abnormalities warrants additional investigation.

### Conclusion

In conclusion, early preterm birth and its associated complications, including IVH and PHHP, remain a serious global challenge to infant and childhood mortality and morbidity. The findings reported here address several of the needs identified in a recent NIH-sponsored symposium on hydrocephalus (McAllister et al., 2015). The age-appropriate, clinically relevant model of PHHP induced by prenatal CAM plus P1 IVH provides a means to test mechanisms and potential interventions to modulate hydrocephalus in neonates. Our results support the use of DTI as an imaging biomarker of injury and repair for PHHP. Most importantly, use of a clinically viable regimen of endogenous neuroprotective agents EPO and MLT to prevent PHHP suggests that safe, economically sound, non-surgical treatments for hydrocephalus may be possible to transform the care of preterm infants with severe IVH in the near future.

### Example 3: Repetitive Neonatal Erythropoietin and Melatonin Combinatorial Treatment Provides Sustained Repair of Functional Deficits in a Rat Model of Cerebral Palsy

Cerebral palsy (CP) is the leading cause of motor impairment for children worldwide and results from perinatal brain injury (PBI). To test novel therapeutics to mitigate deficits from PBI, we developed a rat model of extreme preterm birth (<28 weeks of gestation) that mimics dual intrauterine injury from placental underperfusion and chorioamnionitis. We hypothesized that a sustained postnatal treatment regimen that combines the endogenous neuroreparative agents erythropoietin (EPO) and melatonin (MLT) would mitigate molecular, sensorimotor, and cognitive abnormalities in adults rats following prenatal injury. On embryonic day 18 (E18), a laparotomy was performed in pregnant Sprague-Dawley rats. Uterine artery occlusion was performed for 60 min to induce placental insufficiency via transient systemic hypoxia-ischemia, followed by intra-amniotic injections of lipopolysaccharide, and laparotomy closure. On postnatal day 1 (P1), approximately equivalent to 30 weeks of gestation, injured rats were randomized to an extended EPO+MLT treatment regimen, or vehicle (sterile saline) from P1 to P10. Behavioral assays were performed along an extended developmental time course (n=6-29). Open field testing shows injured rats exhibit hypermobility and disinhibition and that combined neonatal EPO+MLT treatment repairs disinhibition in injured rats, while EPO alone does not. Furthermore, EPO+MLT normalizes hindlimb deficits, including reduced paw area and paw pressure at peak stance, and elevated percent shared stance after prenatal injury. Injured rats had fewer social interactions than shams, and EPO+MLT normalized social drive. Touchscreen operant chamber testing of visual discrimination and reversal shows that EPO+MLT at least partially normalizes theses complex cognitive tasks. Together, these data indicate EPO+MLT can potentially repair multiple sensorimotor, cognitive, and behavioral realms following PBI, using highly translatable and sophisticated developmental testing platforms.

### Introduction

Cerebral palsy (CP) is the leading cause of motor impairment for children worldwide and typically results from perinatal brain injury (PBI) (1, 2). While preterm birth is a common etiologic antecedent, motor impairment and associated deficits can also arise from other insults to the developing central nervous system (CNS), including trauma and stroke. Notably, the scope of PBI has shifted over recent decades as more preterm infants survive (3-5), and the proportion of children with more severe motor impairment has increased in the USA (6). Within subpopulations of neonates with PBI, multiple injury mechanisms have been implicated, and emerging evidence strongly suggests that each newborn suffers a unique vulnerability to CNS injury from a combination of (1) inflammation from prenatal infection and/or hypoxia-ischemia (HI); (2) individualized risk from genetic and/or congenital predisposition and acquired prenatal exposures to drugs, toxins, and nutritional status; and (3) postnatal stresses, such as sepsis and surgery. Indeed, intrapartum events are implicated in the etiology of less than 12% of children with CP (7). Thus, there is an urgent need for safe, effective interventions for PBI, and subsequent CP and related deficits.

Infection and HI catalyze PBI by creating a toxic in utero and neural microenvironment that limits oxygen exchange and propagates inflammation during critical periods of neurodevel-opment (8-15). Typically, infants with PBI present with injury to major white and gray matter structures that leads to reduced connectivity of developing cerebral networks. Subsequently, diverse functional deficits ensue with impairment in multiple motor, cognitive, and behavioral realms that precipitates poor educational progress during childhood (16-25). Chorioamnionitis (infection/inflammation of the amniotic fluid, membranes, and placenta) affects placental permeability and blood flow, facilitates HI and fetal transmission of inflammation, and is associated with a significant increase in systemic inflammation (26-29). Chorioamnionitis is common in both preterm and term infants (23, 24, 29-34). It affects approximately 40-80% of very preterm deliveries and 20-34% of deliveries at term (30, 33, 35). Chorioamnionitis is also recognized in as many as 42% of placentas from unremarkable pregnancies (29, 36). Notably, in term infants with HI encephalopathy, the presence of chorioamnionitis predicts decreased responsiveness to hypothermia treatment (8-10, 32, 37, 38) and magnesium sulfate (39). Because these current strategies for neonatal repair are less effective in the setting of chorioamnionitis, we sought to address this unmet need by testing promising neuroreparative agents using a preclinical model of chorioamnionitis.

Despite the wealth of epidemiological and clinical data related to chorioamnionitis and the development of motor deficits in children born preterm, little progress has been made in identifying interventions that mitigate the CNS injury that leads to CP. Indeed, ambulation, behavior, and cognition are complex tasks impacted by early CNS injury (40). To minimize deficits and optimize outcomes for children with CP, novel therapies are required to restore motor skills, sensation, behavior such as attention and social interaction, and cognition, including executive function. However, few novel therapies have directly addressed these complex and compound deficits, particularly the functional pillars of cognition and behavior with motor impairment. Here, we studied a combination therapy of the endogenous neuroreparative agents erythropoietin (EPO) and melatonin (MLT) in an established preclinical rat model that accurately encompasses the complete maternal-placental-fetal brain axis with intrauterine injury and recapitulates pathophysiology from extreme preterm birth. We chose a cocktail strategy to mitigate the multiple patho-physiological mechanisms that contribute to PBI, capitalize on innate CNS recovery, and respond to clinical recommendations on utility of single therapies (41-43). Furthermore, data from our labs and others, confirm combinatorial therapy with EPO+MLT may provide enhanced, synergistic neurorepair by (1) optimizing the genesis and survival of multiple neural cell lineages, including cells with high bioenergetic demands, such as oligodendrocytes, myelin sheaths, and ependyma with motile cilia, (2) normalizing excess calpain activity and its destruction of essential molecules during neurodevelopment, (3) reducing neuroinflammation and free radicals, and (4) limiting mitochondrial dysfunction and associated endoplasmic reticulum stress (44-55). Given this unique avenue for translation and targeted mechanisms of action, we tested the hypothesis that an extended postnatal EPO+MLT cocktail would mitigate gait, sensorimotor, cognitive, and behavioral changes associated with PBI, using highly translatable and sophisticated testing platforms that are similar to the ones used in humans, including digital gait analysis and touchscreen cognitive testing.

### Materials and Methods

The Institutional Care and Use Committee at the University of New Mexico Health Sciences Center, Boston Children's Hospital and Johns Hopkins University approved all experimental procedures. For each experiment described, equal numbers of male and female pups were used, and data represents true n (individual pups) from at least two different dams per condition. Specifically, we adhered to accepted standards for rigorous study design and reporting to maximize the reproducibility and translational potential of our findings, as described by Landis et al. and in the ARRIVE guidelines (56-58). Animals of both sexes were randomized to experimental or sham control groups and EPO+MLT or vehicle treatments. All investigators were blinded to injury and treatment group during the conduct and analyses of each experiment. For each experiment, a power analysis was also performed to estimate the required sample size (G*Power 3.1.9.3). For these calculations, we used published and preliminary data to define the expected means and SDs for each group, and we exceeded the calculated number needed in every experiment. Separate cohorts of rats were used for open field, gait and social interaction, and touchscreen assessments.

### In Utero Injury: Chorioamnionitis

As placental structure and function is of significant clinical importance to neurologic sequelae in preterm survivors (59-61), we use a prenatal model of in utero transient systemic HI (TSHI) and intra-amniotic lipopolysaccharide (LPS) administration in pregnant rats (62-64). This approach capitalizes on an intact maternal-placental-fetal unit and is a model of PBI from extreme preterm birth (<28 weeks of gestation) that mimics dual intrauterine injury from placental underperfusion and chorioamnionitis (65). Briefly, under isoflurane anesthesia, a laparotomy is performed on embryonic day (E) 18. Uterine arteries are clamped for 60 min and followed by intra-amniotic injections of LPS (4 μg/sac; 0111:B4, Sigma, St. Louis, MO, USA) (62, 64, 65). Sham controls undergo anesthesia and laparotomy for 60 min without arterial clamping or LPS injections. Following closure of the laparotomy, dams receive narcotic pain medication, recover, and pups are born vaginally at E22, approximately equal to 30-32 weeks in human gestation. We have previously reported the effects of TSHI and LPS alone, and in concert, on CNS pathological hallmarks, functional motor outcomes, histologic placental injury, and expression of common pro-inflammatory cytokines (63, 64).

### EPO and MLT Combination Therapy

Erythropoietin and MLT are endogenous, developmentally regulated molecules that are individually most effective for neurorepair when administered in extended dosing regimens (45, 48, 66-6 L Rodents are born at a time equivalent to the human third trimester, with P9 approximately equivalent to term in human gestation (70). Accordingly, we used an established, clinically relevant dosing regimen (47, 48, 55, 71), in which pups on postnatal day (P) 1 from all injured litters were individually randomized to receive either EPO (2,000 U/kg, R&D Systems, Minneapolis, MN, USA) plus MLT (20 mg/kg, Sigma), or vehicle (sterile saline). Subsequently, EPO was then administered intraperitoneally once daily from P1 to P5 and MLT was administered once daily from P1 to P10, comparable to dosing regimens used in human neonatal trials. When EPO was administered in isolation, it was given from P1 to P5 at 2,000 U/kg/dose as previously published (47, 48, 54, 55, 71, 72). Prior work has shown that shams do not exhibit any negative effects from EPO and MLT treatment (55), and thus to conserve resources, shams received only vehicle.

### Open Field

A circular open field arena (100 cm diameter) was placed in a quiet, well-lit room (130 lm), and was marked to divide the arena into three equally spaced, concentric circles labeled the center, neutral, and peripheral zones. At P28-P30, each rat was initially placed against the wall of the testing arena and allowed to explore for 15 min. Anymaze™ video-tracking software was used to record and measure open field behavior.

### Gait Analysis

Computerized gait analysis was performed on P25-P26 as previously described (62, 71). Briefly, digital video of each rat running on a backlit transparent treadmill set at 30 cm/s was acquired with a high-speed camera and analyzed using Digigait software (Mouse Specifics, Framingham, MA, USA). Digigait software analyses identifies individual paw prints and allows calculation of multiple gait metrics and kinematic measurements based on the position, area, and timing of each step. In utero chorioamnionitis induces a global injury. Thus, data from right and left hindlimbs were combined for analysis.

### Social Interaction

A standard paradigm was used to identify impaired social interaction in rats at P30-P32 (73-75). Briefly, 1 h prior to testing, each rat was isolated in a clean cage. For social interaction testing, two rats of the same sex and treatment group, but from different litters, were placed in a dimly lit (30 lm) circular testing arena (100 cm diameter) and recorded for 10 min using Anymaze™ video-tracking software. Each pair was counted as one social unit. Two observers blinded to the treatment group independently reviewed the trials and scored periods of social interaction (trailing, sniffing, grooming, playing, etc.). Intraclass coefficient was calculated for interrater reliability of social scoring. Olfactory testing for social and food odors confirmed primary sensory deficits were not related to the impaired social interaction observed in the injured animals.

### Touchscreen Testing

To better define deficits in cognitive realms, we use a touchscreen operant platform to test specific components of cognition and executive function commencing with mild food deprivation at P28, training at P35, initial testing at P42, and continuing through completion of the paradigms at approximately P90 (76-80). Briefly, using a separate cohort of rats, operant behavior was tested in a sound and light attenuating chamber (Med Associates, St. Albans, VT, USA). A pellet dispenser delivers 40 mg dustless pellets (Bioserv, Frenchtown, NJ, USA) into a magazine, and a houselight is located at one end of the chamber. The opposite end of the chamber houses a touch-sensitive screen (Conclusive Solutions, Sawbridgeworth, UK) overlaid by a black acrylic aperture plate, resulting in two separate touch areas for the rat to register a response. Stimulus presentation in the response windows and touches were controlled and recorded by KLimbic Software (Conclusive Solutions).

### Pretraining

On P28, rats were first slowly reduced and then maintained at 85% free-feeding body weight. Rats were weighed and assessed for general health daily. The mild weight reduction was well tolerated. Prior to training, rats were acclimated to the 40 mg food pellet reward by provision of 25 pellets/rat in the home cage. Rats were then habituated to the operant chamber and to eating from the pellet magazine. Rats retrieving at least 48 pellets in 60 min were moved to a 4-stage training regimen. Rats first performed autoshaping, followed by three visual discrimination training sessions (76-79).

### Discrimination and Reversal Learning

Following pretraining, all rats were tested on a pairwise discrimination-reversal paradigm during daily 60 min sessions. For discrimination learning, 2 novel, equiluminescent stimuli verified for rats, were presented in a spatially pseudo-randomized manner over 60-trial sessions (5-s inter-trial interval) (76-80). Responses at one stimulus yielded a reward, whereas responses at the other stimulus resulted in a 5 s time-out (singled by extinguishing the house light). Designation of initially reward stimulus was randomized across treatment. Stimuli remained on screen until a response was made. Rats were trained to an a priori criterion of greater than ≥80% correct responses for two consecutive days. Assessment of reversal learning began on the session after dis-crimination criterion was attained. For this test, the designation of stimuli as correct versus incorrect was reversed for each rat. Like discrimination, rats were tested on 60-trial daily sessions for reversal to an a priori criterion of ≥80% correct responses for two consecutive sessions. Errors on first presentation reversal trials were followed by correction trials which continued until a correct response was made, or the session ended. Failing criteria was set a priori at 21 sessions (days) for visual discrimination and 21 sessions (days) for reversal.

We recorded the following dependent measures during discrimination and reversal: total sessions, correct responses made, errors (incorrect responses made), correction errors (correction trials, reversal only), reaction time (time from stimulus presentation to touchscreen response), and magazine latency (time from touchscreen response to reward retrieval) (81). Discrimination performance was analyzed across all sessions required to reach criterion. To examine distinct phases of reversal (early perseverative and late learning) mediated by cortical and striatal subregions, respectively, we also analyzed errors and correction trials. Assuming a rat would achieve 50% correct by chance, perseveration was defined as sessions where performance was below 50% correct, and learning as performance from 50% correct to passing criterion, as previously described (81-83).

### Statistical Analysis

Statistical analyses were performed using SPSS25 (IBM, Armonk, NY, USA). For all analyses, data are represented as mean±SEM, with p<0.05 considered significant. For analysis of sham, vehicle-treated injury and EPO+MLT-treated injury groups, all parametric variables were tested for normal distribution with the Shapiro-Wilk test with Levene's test to confirm homogeneity of variances. A two-way ANOVA was then performed with Bonferroni's post hoc correction for multiple comparisons. For non-parametric variables such as passing criteria in touchscreen testing, a Kruskal-Wallis test with Dunn's post hoc correction was performed.

### Results

### EPO+MLT Mitigates Disinhibition Following Prenatal Injury

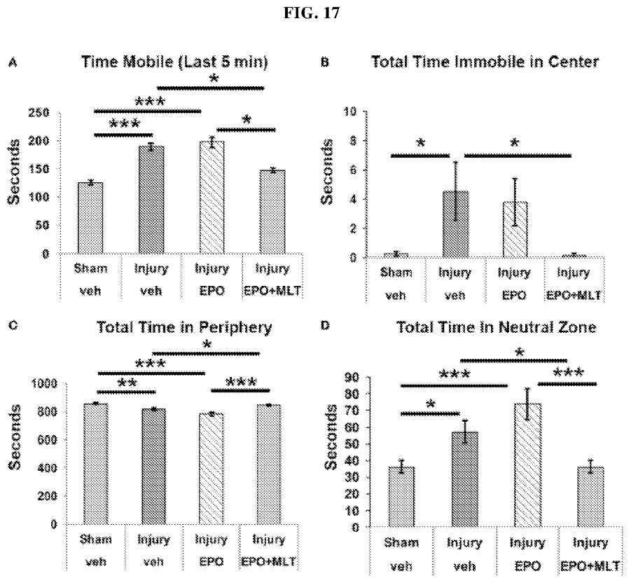

We assessed open field behavior to quantify activity and disinhibition. Compared to sham controls (n=29), rats subjected to prenatal injury (n=23) were much more mobile, which was particularly evident in the last 5 min of the 15-min observation period (FIG. 17A). Interestingly, compared to vehicle-treated rats with prenatal injury, EPO+MLT normalized the hyper-mobility (n=28, two-way ANOVA, p=0.022), whereas EPO alone (n=15) did not. After prenatal injury, adult rats were also disinhibited. Specifically, those with in utero injury showed a lack of environmental awareness by spending more time immobile in the arena center compared to shams (p=0.03; FIG. 17B). Similarly, prenatal injury had a significant effect on disinhibition, with sham rats exploring the peripheral zone for significantly longer periods compared to vehicle-treated injury rats (p=0.001; FIG. 17C), and spending less time in the neutral zone (p=0.041; FIG. 17D). Treatment with EPO+MLT, but not EPO alone, normalized total time spent in the peripheral (p=0.024) and neutral zones (p=0.038; FIG. 17), consistent with typical rat behavior, appropriate anxiety and general avoidance of open areas.

### EPO+MLT Normalizes Hindlimb Deficits After Prenatal Injury

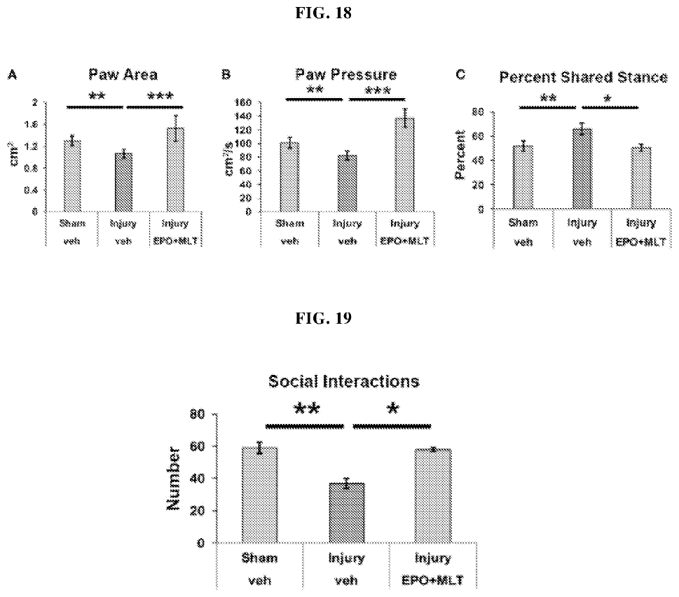

After observing that EPO+MLT normalized hyperlocomotion and disinhibition in adult rats following prenatal injury, we performed a detailed computerized digital analysis of gait to determine if EPO+MLT could improve motor performance. Compared to shams (n=18), after prenatal injury vehicle-treated adult rats exhibit an abnormal gait, stance and paw placement, with decreased paw area contact (n=21, p=0.012) and decreased paw pressure (p=0.031) suggestive of toe-walking, and reduced percent shared stance (n=21, p=0.006) (FIG. 18) consistent with spastic gait patterns observed in ambulatory children with CP. Significantly, neonatal combination therapy with EPO+MLT (n=7) reverses deficits in stance (p=0.035) and paw placement (area and pressure both p<0.001), consistent with an improved gait kinematic efficiency with combination treatment.

### EPO+MLT Attenuates Deficits in Social Interaction

To quantify social interaction, pairs of sex, injury, and treatment-matched rats from different litters were observed and scored. The interrater reliability of social interaction scoring was 0.932. Sham (n=18 rats in 9 pairs, p<0.001) and injured rats treated with EPO+MLT (n=8 rats in 4 pairs, p=0.007) had significantly more social interactions during the observation period, including sniffing, playing, and grooming, compared to vehicle-treated rats with in utero injury (n=14 rats in 7 pairs) (FIG. 19). Significantly, EPO+MLT treatment ameliorated deficits in social drive and behavior.

### EPO+MLT Mitigates Deficits in Executive Function

To complement our assessment of gait, open field, and social behavior in adult rats with in utero injury, we completed a sophisticated assessment of visual discrimination and reversal learning in our animals to evaluate executive function. We began by validating the touchscreen platform in our model of in utero chorioamnionitis and assessed whether adult rats following prenatal insult could perform visual discrimination. Importantly, rats in all three treatment groups were successful in completing all aspects of touchscreen habituation and training.

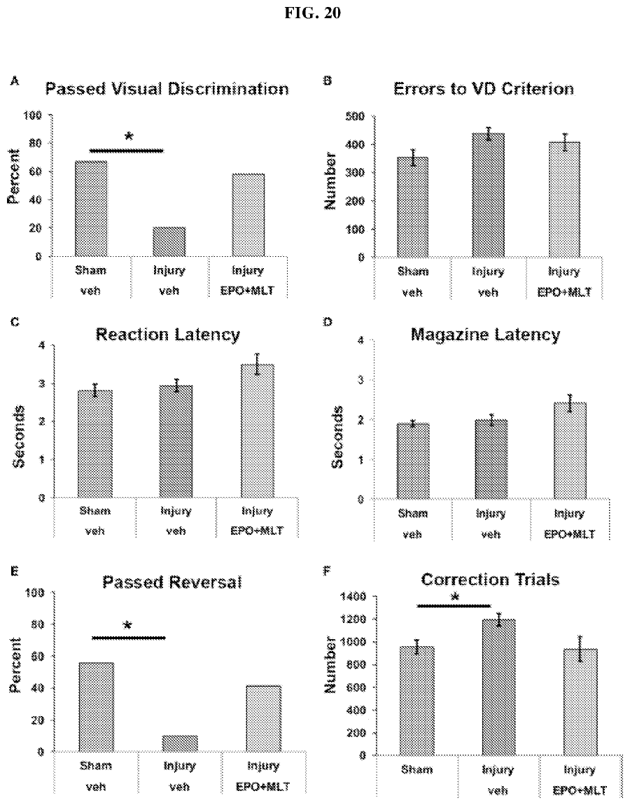

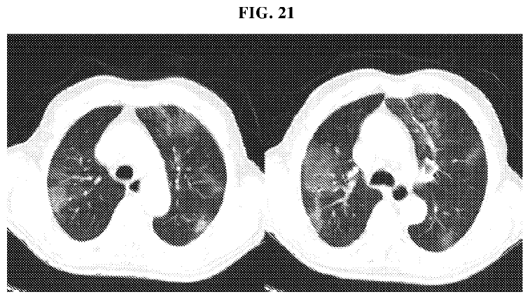

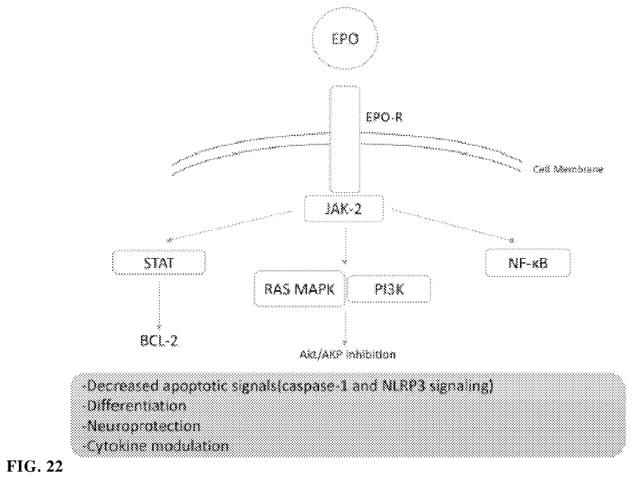

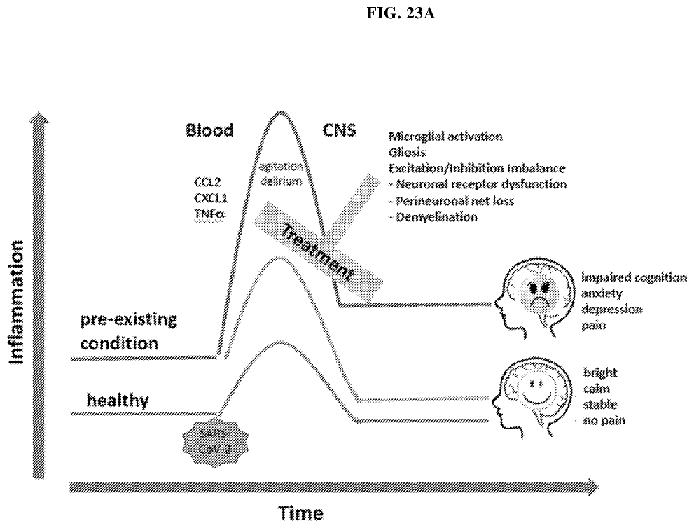

We first assessed cognitive performance on visual discrimination. Rats from each experimental group were able to perform VD, with 67% of sham (n=27) and 20% of vehicle-treated injured rats (n=20, p=0.005) achieving passing criteria, compared to 58.3% of injured rats treated with EPO+MLT (n=12, p=0.11; FIG. 20A). After assessing overall performance and pass rate, we then analyzed the number of errors throughout the visual discrimination paradigm as a more rigorous and granular metric of task performance. Notably, for those rats completing VD, a similar number of errors to achieve passing criteria was noted across injury and treatment groups (FIG. 20B). As expected, all rats had comparable reaction time and magazine latency (FIGS. 20C, 20D) throughout the VD paradigm.

Upon successful completion of VD, rats were evaluated for reversal learning. Vehicle-treated injured rats were significantly impaired and fewer passed the overall learning paradigm compared to sham and EPO+MLT-treated rats. Specifically, only 10% of vehicle-treated injured animals successfully passed criteria for VD and reversal (p=0.046) compared to 55.5% of sham and 41.5% of injured animals treated with EPO+MLT (p=0.07; FIG. 20E). Notably, injured animals treated with vehicle required more correction trials compared to shams (p=0.034). Injured rats treated with EPO+MLT showed a trend toward fewer correction trials (p=0.077), compared to vehicle-treated rats (FIG. 20F). Further analyses of the maladaptive learning in the reversal paradigm consistent with a lack of cognitive flexibility, indicated a trend for improved performance during both perseveration and learning phases of the reversal paradigm. These results show that touchscreen testing can be used in PBI to distinguish complex behavior related to executive function and learning and that EPO+MLT can at least partially reverse the reduced cognition present after prenatal injury.

### DISCUSSION

In this investigation, we tested the efficacy of combined EPO+MLT for neurorepair of the deficits associated with CP using translatable outcome measures, with the goal of facilitating rapid transition to neonatal clinical trials. To begin, we capitalized on a preclinical platform and model of CP that accurately recapitulates the multi-faceted pathophysiology of early CNS injury, including an intact maternal-placental-fetal axis, and sustained deficits in adult animals in multiple functional domains. These investigations reflect recent clinical epidemiological progress that indicates most CP arises from prenatal injury and that only 12% of cases arise from intrapartum insults (7). Consistent with clinical data, the preclinical findings reported here reaffirm the concept that chorioamnionitis concomitant with placental insufficiency results in dynamic, multifactorial, and permanent changes to the CNS culminating in functional deficits in mature rats. Indeed, this prenatal insult causes significant chronic behavioral, social, executive function, and gait deficits in adult rats, similar to those observed in children with CP (46, 62, 71). This model that incorporates intrauterine chorioamnionitis is one of very few preclinical models to induce persistent gait and neurocognitive deficits in the mature CNS (40, 70).

Mechanistically, compelling evidence suggests EPO and MLT have significant promise as potential synergistic interventions for neonates at high risk of CP. EPO and MLT have multiple over-lapping and complementary mechanisms of action. As developmentally regulated growth molecules, both EPO and MLT enhance neuronal and oligodendroglial survival and differentiation after CNS injury, suppress toxic cell death pathways, reduce free radicals, and mitigate inflammation from neonatal CNS infection (44-55). Unlike EPO, which is produced predominantly by the kidney and neural cells after birth (84), MLT is an endogenous indoleamine that is produced by the pineal gland

postnatally and classically reported to regulate circadian rhythms. It is a direct antioxidant and free radical scavenger and also has indirect actions to increase the production of antioxidant enzymes including glutathione peroxidase (GP) and superoxide dismutase (SOD) (85). Notably, preterm infants with PBI, including those initiated in utero by choriomanionitis, are known to have reduced levels of GP and SOD in both the brain and lung (86).

The combination of EPO and MLT also has direct anti-inflammatory and immunomodulatory properties integral to their beneficial effects. Impaired regulation of immune responses is detrimental to multiple pregnancy outcomes, including preterm birth. It is plausible that both EPO and MLT link maternal, placental, and fetal physiological cell signaling through mechanisms of entrainment and direct biological actions (87). Interestingly, MLT is synthesized in higher concentrations within the placenta than the pineal gland (87, 88). Specifically, cyto- and syncytio-trophoblasts from the placenta contain two enzymes, serotonin N-acetyltransferase and N-acetylserotonin methyltransferase, which metabolize serotonin to MLT. Once in the circulation, MLT can increase phagocytosis, antigen presentation, and exert antioxidant effects (87, 89). Indeed, both EPO and MLT are known to affect Th1/Th2 ratio, Th17, neutrophils, and microglia, major cellular mediators of chorioamnionitis and PBI (87, 90). Through separate signal transduction, CNS inflammation actually reduces innate CNS EPO and MLT production, thereby diminishing endogenous neurorepair (91). In this context, similar to exogenous EPO therapy, exogenous MLT administration after birth may supplement innate levels by the pineal gland and replace a MLT deficit arising from premature separation from placental sources and/or induced inflammation from intrauterine infection or injury.

Erythropoietin and MLT also promote the genesis, survival, and differentiation of neural cells in the developing and mature CNS and reduce calpain-mediated injury. Previously, we have shown that sustained excess calpain activity is an important mechanism of injury in the immature CNS (45, 47, 48) and that extended EPO treatment mitigates calpain-mediated damage (48). Specifically, calpain degrades CNS molecules and proteins essential for the formation of cerebral circuits, including neurofilaments, myelin basic protein, and the potassium chloride cotransporter KCC2 (48). Therefore, through EPO and MLT together, it may be possible to cumulatively preserve more axon-myelin structural units, including those in major cerebral white matter and corticospinal tracts, by inhibiting detrimental protease expression, preserving structural connectivity, and restoring inhibitory neural networks. Indeed, it is through this action on structural and functional connectivity, neural conduction, and excitatory/inhibitory balance of fundamental circuitry that this combination of therapy likely improves motor and cognitive function into early adulthood (P90) following prenatal injury (46-48, 71).

Recently, it has been demonstrated that EPO has an additional novel mechanism in regulation of homeostatic plasticity and synaptic strength (92). Together, with previous reports on the modulation of inhibitory circuitry in brain regions key to higher order brain function and structural connectivity, this effect on synapses provides an additional novel molecular mechanism supporting the improvement in cognition and behavior shown here, and the normalization of the trajectory of brain development after perinatal injury (47, 66, 71, 72, 92-94). Similarly, complimenting the EPOR distribution on glia, and neurons, and the importance of receptor/ligand balance in the developing brain (55), MLT receptors, MT1 and MT2, are present in regions of the brain that are important to cognition and memory, including the hippocampus and frontal cortex and are similarly regulated by endogenous MLT (69, 87, 95, 96). In rodents, several studies have reported improved social behavior, anxiolytic, antidepressant, and memory-facilitating effects of exogenous MLT related to modulation of essential neurotransmitters and their receptors, including GABAergic, dopaminergic, glutamatergic, cholinergic, and noradrenergic transmission (69, 97 Consistent with our data, studies in mice confirm a MLT-induced decrease in open field hyperlocomotion (69). Indeed, our data also matches prior studies demonstrating that MLT administration in similar dosing ranges reverses ketamine-induced deficits in social interaction and memory impairment (69). Significantly, these investigators found that mitigation of social deficits only occurred with dose-dependent, repeated administration of MLT. Interestingly, in our studies, the combination of EPO+MLT was able to reverse abnormal open field behavior, whereas EPO alone was not. This highlights that, in specific microenvironments, a combination of agents may be more effective than one agent alone, and through repair of white matter, synapses, structural connectivity, neural network efficiency, and multiple neurotransmitter systems, EPO and MLT together may additively improve multiple pillars of cognition and behavior.

This is the first time to our knowledge that touchscreen platforms have been used with preclinical models of CP and PBI. Given that many children with CP have sensorimotor and cognitive deficits, there is a need to study strategies that address both functional domains. Touchscreen assessment is a highly translatable outcome measure also utilized in human trials and the Cambridge Neuropsychological Test Automated Battery is regularly used for neuropsychological testing of children and adults (76, 80). Our results demonstrate that EPO+MLT partially mitigates deficits of cognition, specifically executive function and reversal learning. These data support the clinical literature showing that very preterm children and adolescents are at high risk for executive function deficits that only become apparent with increasing cognitive demands. Specifically, compared to healthy born peers, preterm adolescents scored significantly lower in the most demanding levels of working memory, planning, cognitive flexibility, and verbal fluency tasks, despite no group differences being detected at lowest demand levels (101).

We also found in utero injury influenced exploratory behavior in mature animals, with EPO+MLT normalizing excess center mobility and resting time in an open field. These data are consistent with prior studies demonstrating improvements in exploratory behavior and resting time with MLT treatment, consistent with normalization of disinhibition, hyperlocomotion, and depression-like behavior (102). Indeed, prior reports suggest that MLT exerts a long-term effect on striatal dopamine content by enhancing monoamine synthesis (102, 103). While that investigation was performed in older rats, it is possible that normalized monoamine system development might improve motor coordination and cognition. Similarly, MLT also acts as a 5HT2A antagonist in the hippocampus, and through the regulation of 5HT release may also impact complex behaviors related to anxiety, behavioral inhibition, and locomotion (102, 104). Notably, children with CP often exhibit spasticity and difficulty with selective motor control. Selective motor control is regulated predominantly by descending serotonin pathways that innervate the lumbar spinal cord on E18 in rats (105), the same age as the prenatal injury used here. Importantly, signaling via 5HT2A upregulates spinal KCC2 levels (106). Serotonin signaling is also integral to cognitive flexibility (107). Thus, repairing myelination, axons, KCC2 levels and preserving homeostasis of serotonin signaling may be key mechanistic conversion points of EPO+MLT combination therapy and critical to minimizing spasticity, loss of selective motor control, and preserving cognition in children with CP from prematurity.

In conclusion, EPO+MLT are plausible targeted pharmacotherapies that specifically enhance neurorepair via novel and disease-specific molecular mechanisms. Receptor and non-receptor-mediated pathways underpin the multiple neuroprotective effects of MLT and EPO that include supporting mitochondrial function, and post-injury plasticity, and antioxidant, anti-apoptotic, and anti-inflammatory actions (108, 109). Together with normalization of social interaction, these data suggest a plausible treatment strategy to address multiple realms of cognition and behavior. In conjunction with data presented in this study, coupled with EPO and MLT's known safety profile, multiple beneficial mechanisms of action, ability to penetrate the brain and organelles, and ease of administration, EPO and MLT in combination merits thoughtful consideration of clinical trials for preterm infants with brain injury.

### Example 4: Neuro-Immunomodulation to Ameliorate Post-COVID Syndrome Neurological Symptoms

### Development of Model

As no model of post-COVID syndrome (e.g. PASC) existed, a new preclinical model in rats is developed. Our model is non infectious and uses LPS (bacterial endotoxin) to prime the immune system and then Poly IC to mimic viral illness.

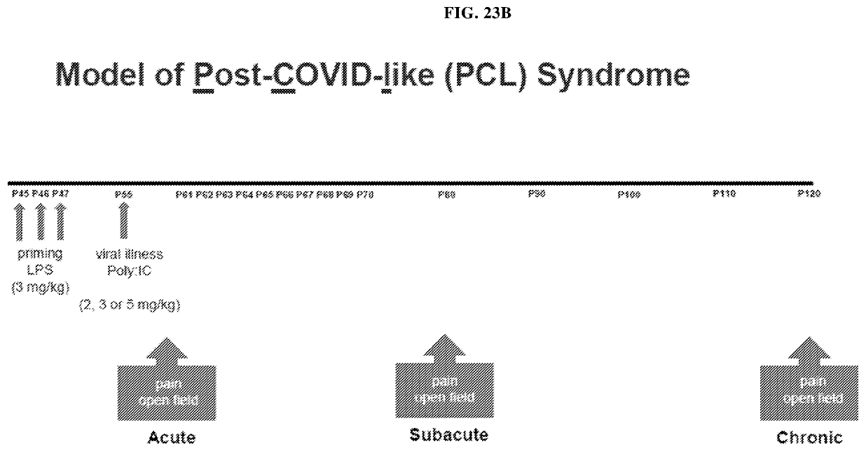

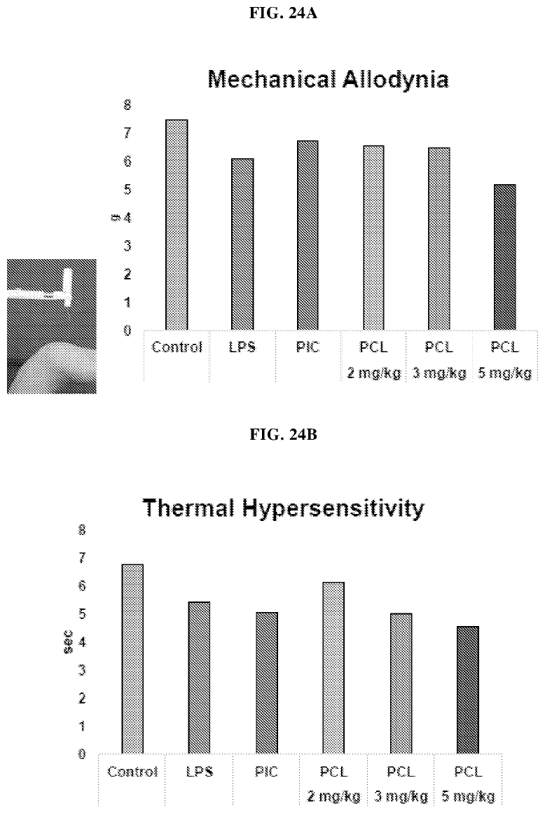

SARS-CoV-2 (COVID) illness is defined by robust systemic and central nervous system (CNS) inflammation that leads to impaired cognition, psychiatric issues and chronic pain in susceptible patients. Neuro-immunomodulatory drug cocktails that mitigate the profound immune cell activation and recruitment, gliosis, loss of perineuronal nets and cerebral white matter provide a novel mechanistically-based treatment strategy for this immense patient population (FIG. 23A).

Exemplary model of Post-COVID-Like (PCL) syndrome is shown in FIG. 23B. LPS (3 mg/kg ip) is given to prime the immune system of rats on postnatal (P) 45, 46 and P47. Subsequently, Poly IC (2, 3, or 5 mg/kg ip) is given on P55 to mimic a viral exposure. The response to these stimuli are studied in the following acute, subacute and chronic timepoints. Pain and activity in an open field are outcome measures we use routinely to track the development of the PCL syndrome.

Currently, there is no preclinical model of Post-COVID-Like (PCL) syndrome. While rodent ACE2 receptors are resistant to SARS-CoV2, systemic LPS (lipopolysaccharide bacterial endotoxin, noninfectious mimic of bacterial infection) may be employed to replicate pre-existing condition and prime immune system, which follows with systemic Poly I:C (Poly I:C—polyinosinic:polycytidylic acid LMW/HMW, noninfectious synthetic analog of double-stranded RNA) to mimic viral illness.

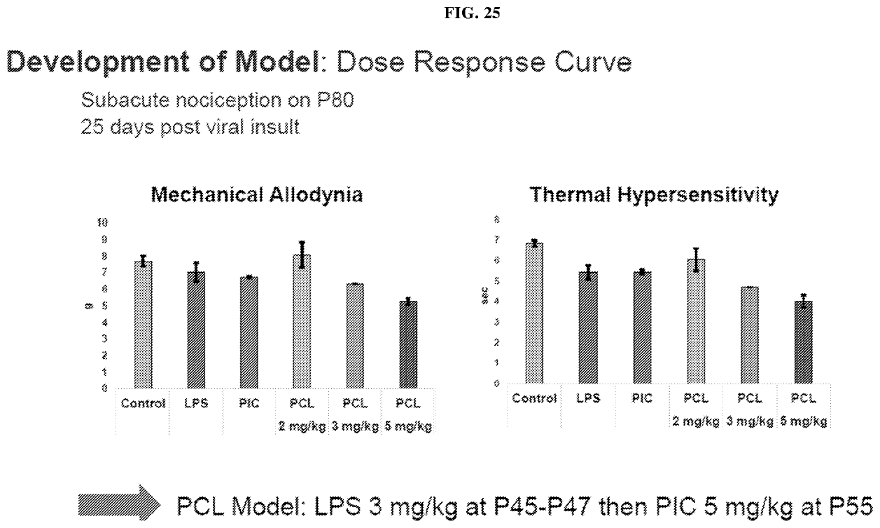

Dose response curve is shown in FIG. 24A (mechanical allodynia) and FIG. 24B (thermal hypersensitivity), which are based on PCL Model (LPS 3 mg/kg at P45-P47 then PIC 5 mg/kg at P55). LPS (3 mg/kg ip) is given to prime the immune system of rats on postnatal (P) 45, 46 and P47. Subsequently, Poly IC (2, 3, or 5 mg/kg ip) is given on P55 to mimic a viral exposure. Notably. PolyIC at 5 mg/kg (dark purple bars) yields the most robust allodynic pain response and thermal hypersensitive phenotype by P80. Poly IC dose response is observed with 2, 3, or 5 mg/kg. indeed, the higher the dose of PolyIC the more pronounced the allodynia and thermal hypersensitivity. Dose response curve is shown in FIG. 25 with subacute nociception on P80 and 25 days post viral insult.

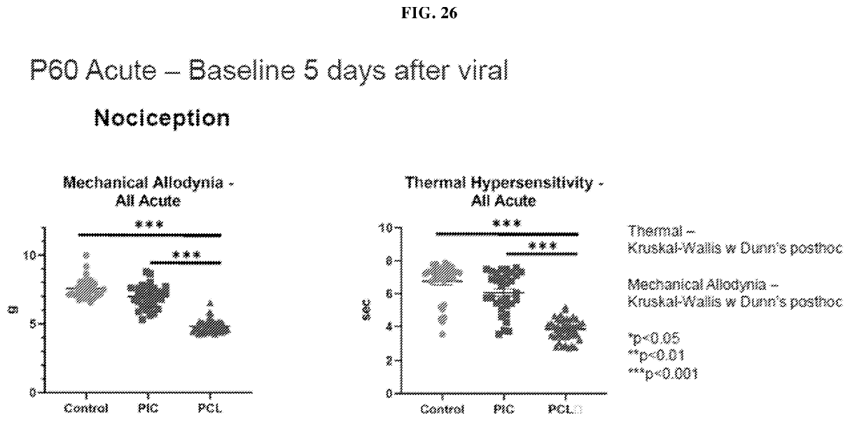

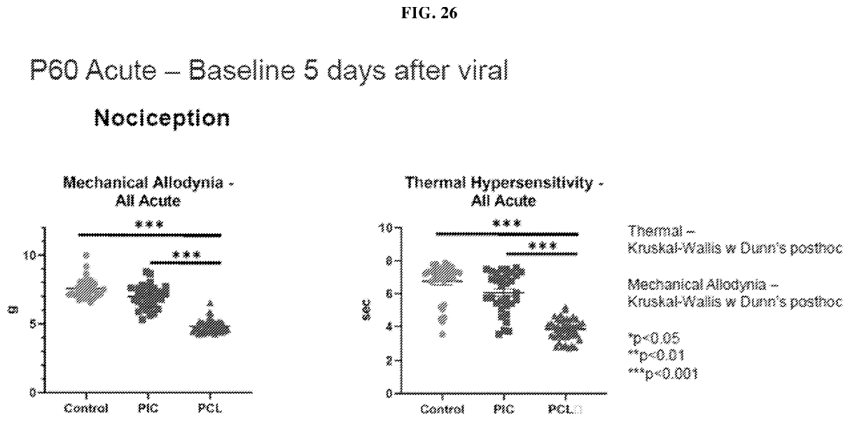

FIG. 26 shows P60 acute results (nociception, baseline 5 days after viral). LPS (3 mg/kg ip) is given to prime the immune system of rats on postnatal (P) 45, 46 and P47. Subsequently, Poly IC (5 mg/kg ip) is given on P55 to mimic a viral exposure. Mechanical Allodynia and Thermal hypersensitivity was then assessed acutely, 5 days after viral exposure. Those rats with PCL syndrome experience more allodynia and more thermal hypersensitivity compared to control rats or those rats only receiving PolyIC.

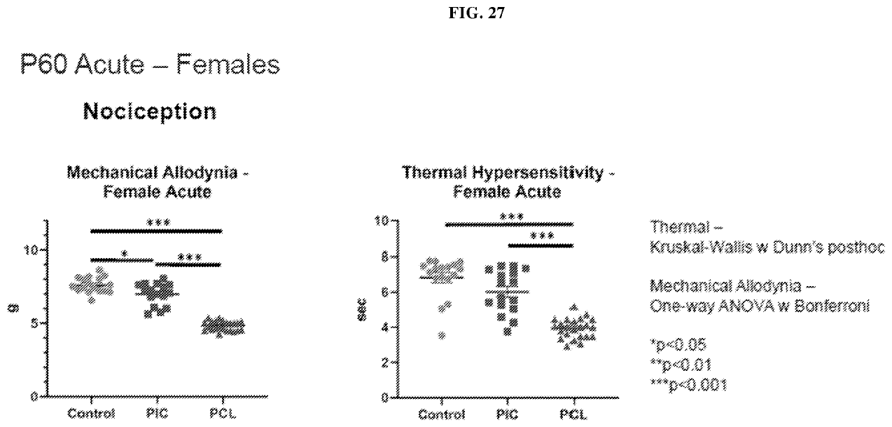

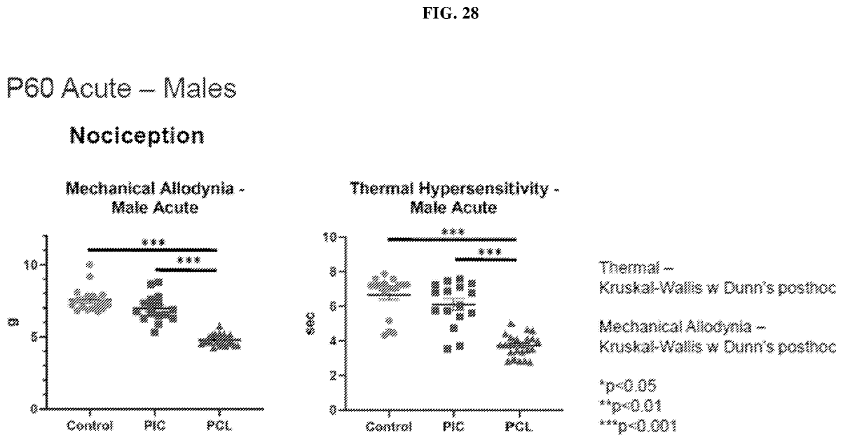

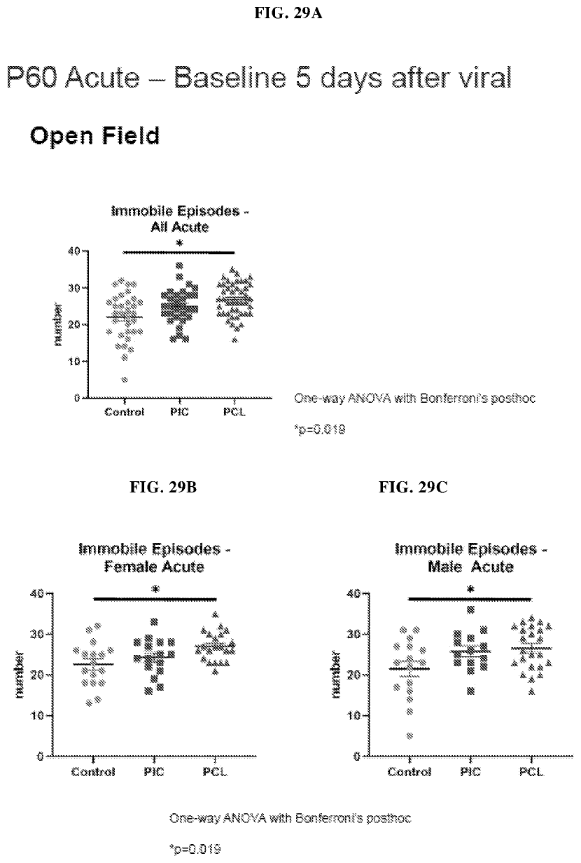

FIG. 27 shows P60 acute results (females) and FIG. 29 shows P60 acute results (males). For acute nociception in females (FIG. 28), LPS (3 mg/kg ip) is given to prime the immune system of rats on postnatal (P) 45, 46 and P47. Subsequently, Poly IC (5 mg/kg ip) is given on P55 to mimic a viral exposure. Mechanical Allodynia and Thermal hypersensitivity was then assessed acutely in female animals, 5 days after viral exposure. Those rats with PCL syndrome experience more allodynia and more thermal hypersensitivity compared to control rats or those rats only receiving PolyIC.

As shown in FIG. 28, acute nociception in males (FIG. 29), LPS (3 mg/kg ip) is given to prime the immune system of rats on postnatal (P) 45, 46 and P47. Subsequently, Poly IC (5 mg/kg ip) is given on P55 to mimic a viral exposure. Mechanical Allodynia and Thermal hypersensitivity was then assessed acutely in male animals, 5 days after viral exposure. Those rats with PCL syndrome experience more allodynia and more thermal hypersensitivity compared to control rats or those rats only receiving PolyIC.

FIG. 29A shows P60 acute results (open field, baseline 5 days after viral). FIG. 29B shows P60 acute results (females) and FIG. 29C shows P60 acute results (males).

As shown in FIG. 29A, activity and mobility is decreased acutely after PCL Syndrome. LPS (3 mg/kg ip) is given to prime the immune system of rats on postnatal (P) 45, 46 and P47. Subsequently, Poly IC (5 mg/kg ip) is given on P55 to mimic a viral exposure. Activity in an open field was then assessed 5 days after viral exposure. Those rats with PCL syndrome have more freezing or immobile episodes and are less active in the open field compared to control rats.

Activity and mobility is decreased in male and female animals acutely after PCL syndrome as shown in FIG. 29B and FIG. 29C. LPS (3 mg/kg ip) is given to prime the immune system of rats on postnatal (P) 45, 46 and P47. Subsequently, Poly IC (5 mg/kg ip) is given on P55 to mimic a viral exposure. Activity in an open field was then assessed 5 days after viral exposure. Those rats with PCL syndrome have more freezing or immobile episodes and are less active in the open field compared to control rats.

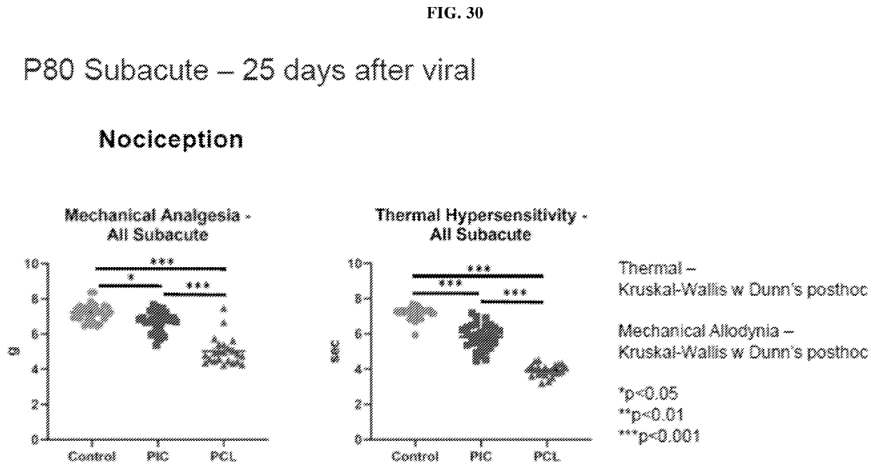

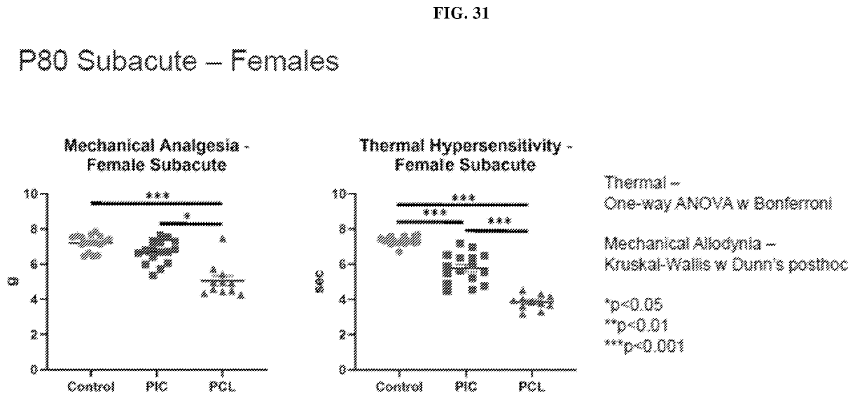

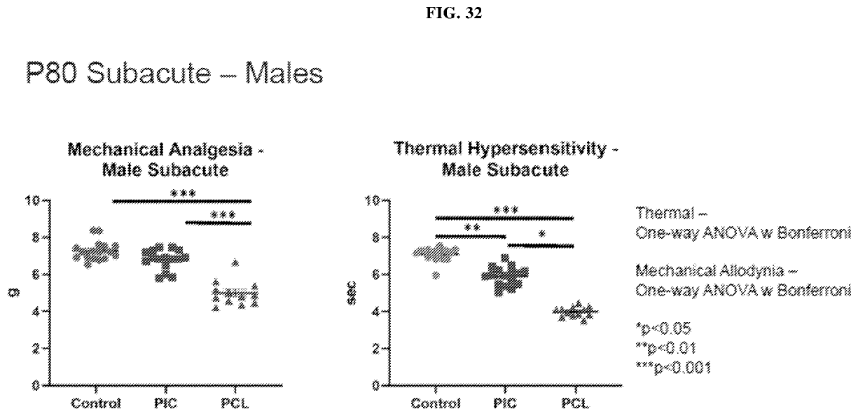

FIG. 30 shows P80 subacute results (nociception, 25 days after viral). For Subacute Nociception in FIG. 31, LPS (3 mg/kg ip) is given to prime the immune system of rats on postnatal (P) 45, 46 and P47. Subsequently, Poly IC (5 mg/kg ip) is given on P55 to mimic a viral exposure. Mechanical Allodynia and Thermal hypersensitivity was then assessed subacutely, 25 days after viral exposure. Those rats with PCL syndrome (purple triangles) experience more allodynia and more thermal hypersensitivity compared to control rats or those rats only receiving PolyIC.

FIG. 31 shows P80 acute results (females) and FIG. 32 shows P80 acute results (males).

For subacute nociception in female animals (FIG. 31), LPS (3 mg/kg ip) is given to prime the immune system of rats on postnatal (P) 45, 46 and P47. Subsequently, Poly IC (5 mg/kg ip) is given on P55 to mimic a viral exposure. Mechanical Allodynia and Thermal hypersensitivity was then assessed subacutely, 25 days after viral exposure. Female rats with PCL syndrome (purple triangles) experience more allodynia and more thermal hypersensitivity compared to control rats or those rats only receiving PolyIC.

For subacute nociception in male animals in FIG. 32, LPS (3 mg/kg ip) is given to prime the immune system of rats on postnatal (P) 45, 46 and P47. Subsequently, Poly IC (5 mg/kg ip) is given on P55 to mimic a viral exposure. Mechanical Allodynia and Thermal hypersensitivity was then assessed subacutely, 25 days after viral exposure. Male rats with PCL syndrome (purple triangles) experience more allodynia and more thermal hypersensitivity compared to control rats or those rats only receiving PolyIC.

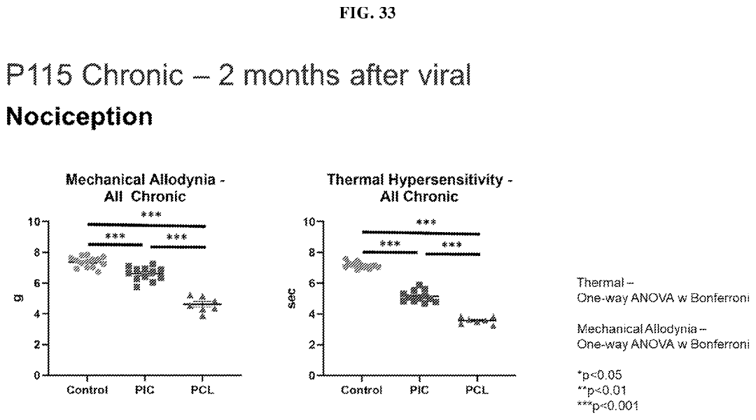

FIG. 33 shows chronic nociception 2 months after vial. LPS (3 mg/kg ip) is given to prime the immune system of rats on postnatal (P) 45, 46 and P47. Subsequently, Poly IC (5 mg/kg ip) is given on P55 to mimic a viral exposure. Mechanical Allodynia and Thermal hypersensitivity was then assessed chronically, 2 months after viral exposure. Those rats with PCL syndrome (purple triangles) experience more allodynia and more thermal hypersensitivity compared to control rats or those rats only receiving PolyIC.

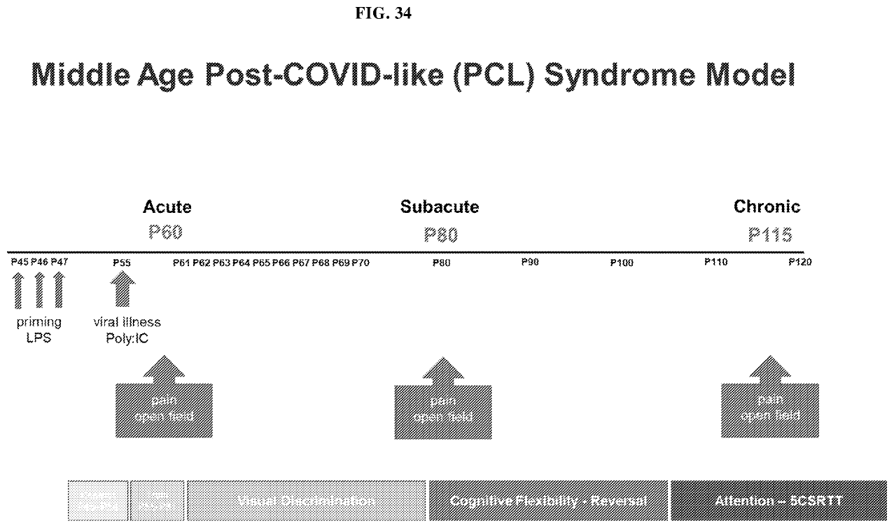

FIG. 34 shows an exemplary experimental design and development of pel syndrome with cognitive assessment. LPS (3 mg/kg ip) is given to prime the immune system of rats on postnatal (P) 45, 46 and P47. Subsequently, Poly IC (5 mg/kg ip) is given on P55 to mimic a viral exposure. The response to these stimuli are studied in the following acute, subacute and chronic timepoints. Pain and Activity in an Open Field are outcome measures we use routinely to track the development of the PCL syndrome. Similarly, Touchscreen cognitive assessment occurs simultaneously throughout the Acute, SubAcute and Chronic Phases. Using a touchscreen assessment platform, we first assess visual discrimination. We then assess cognitive flexibility through a test of reversal learning, and then a test of attention called 5 choice serial reaction (5CSRTT).

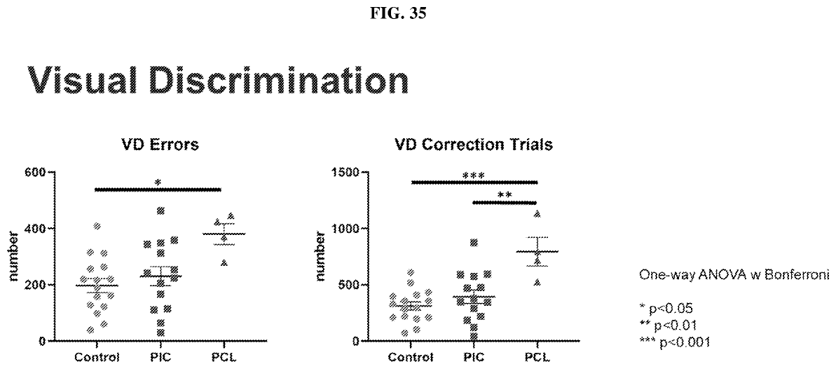

For example, as shown in FIG. 35, Post-COVID Like (PCL) illness yields deficits in cognition. After priming the immune system with systemic lipopolysaccharide on postnatal day 45-47, rats receive the viral mimetic poly(I:C) on P55. Rats with PCL are unable to successfully perform a visual discrimination (VD) test using a touchscreen cognitive platform. The increased number of errors and correction trials required by PCL rats compared to PIC and controls animals is consistent with a deficit of cognition and impaired executive function. (One-Way ANOVA with Bonferroni correction,*p<0.05, **p<0.01, ***p<0.001; PIC=PolyIC; PCL=Post-COVID like which is LPS+PIC).

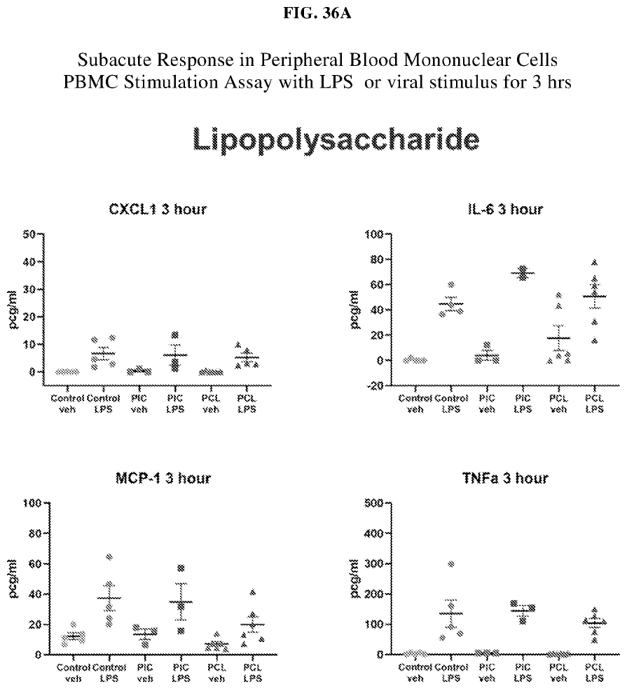

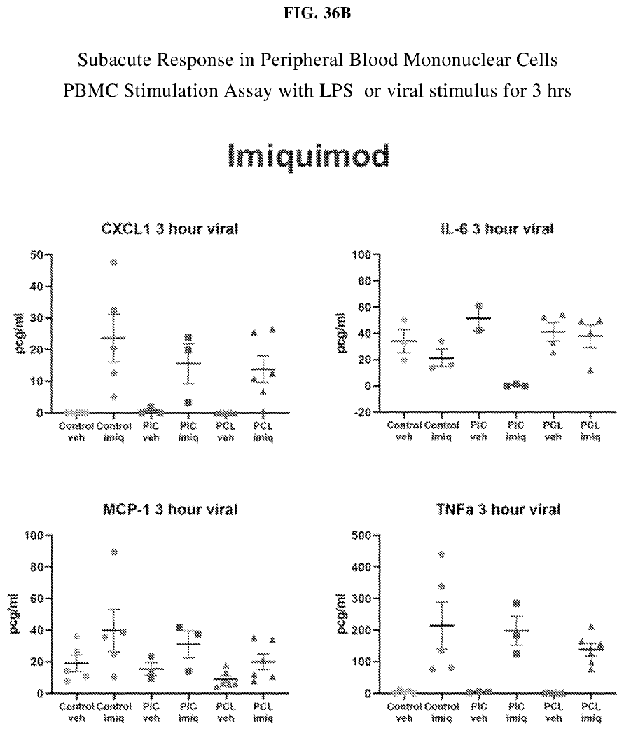

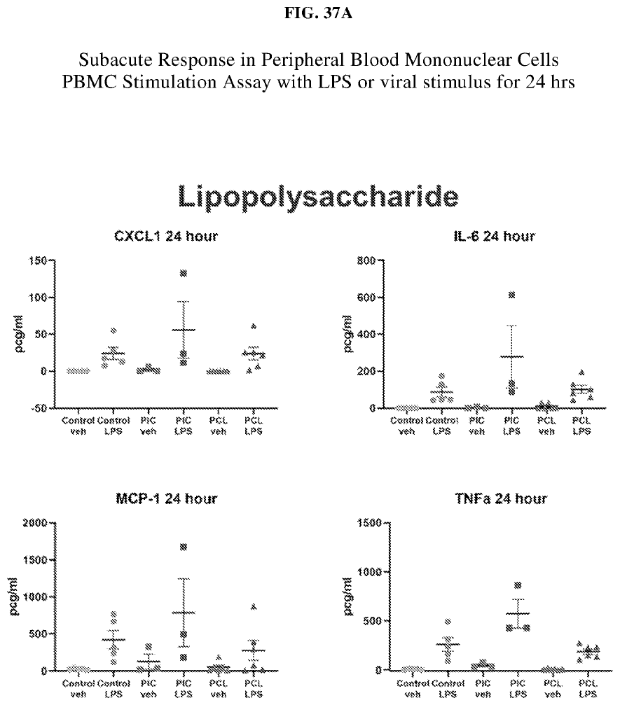

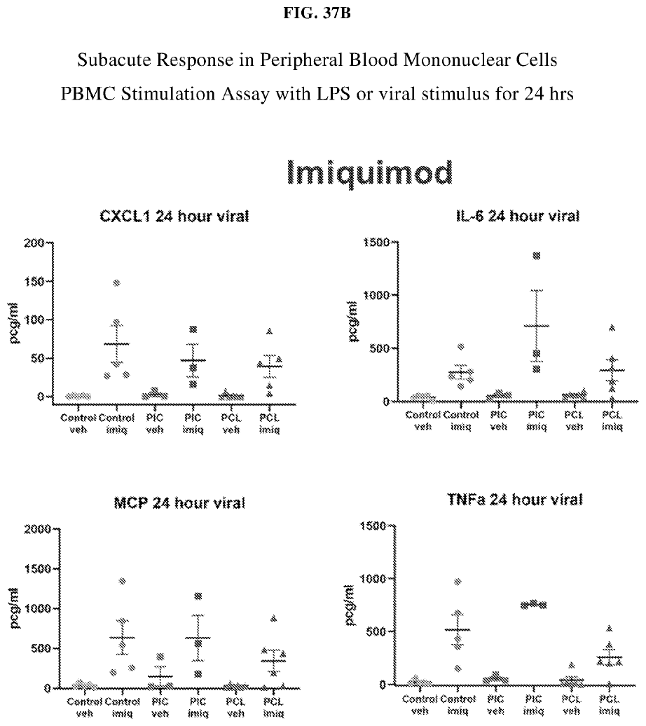

FIGS. 36A-37B show that Post-COVID Like (PCL) illness yields a variable acute inflammatory secretome. After priming the immune system with systemic lipopolysaccharide on postnatal day 45-47, rats receive the viral mimetic poly(I:C) on P55. Peripheral blood mononuclear cells (PBMCs) are harvested and challenged in culture for 3 h with a second hit of LPS (TLR4 agonist) or Viral Stimulant (Imiquimod, TLR7 agonist). Similar to patients with Post-COVID syndrome, secondary bacterial or viral challenges reveal an increased immune response defined by increased chemokines (CXCL1, MCP-1) and cytokines (IL-6 and TNFa) in the secretome. (PIC=PolyIC; PCL=Post-COVID like which is LPS+PIC).

Moreover, FIG. 37A-37B show that Post-COVID Like (PCL) illness yields a variable chronic inflammatory secretome. After priming the immune system with systemic lipopolysaccharide on postnatal day 45-47, rats receive the viral mimetic poly(I:C) on P55. Peripheral blood mononuclear cells (PBMCs) are harvested and challenged in culture for 24 h with a second hit of LPS (TLR4 agonist) or Viral Stimulant (Imiquimod, TLR7 agonist). Similar to patients with Post-COVID syndrome, secondary bacterial or viral challenges reveal an increased immune response defined by increased chemokines (CXCL1, MCP-1) and cytokines (IL-6 and TNFa) in the secretome.

### Middle Age Post-COVID-Like (PCL) Syndrome Treatment

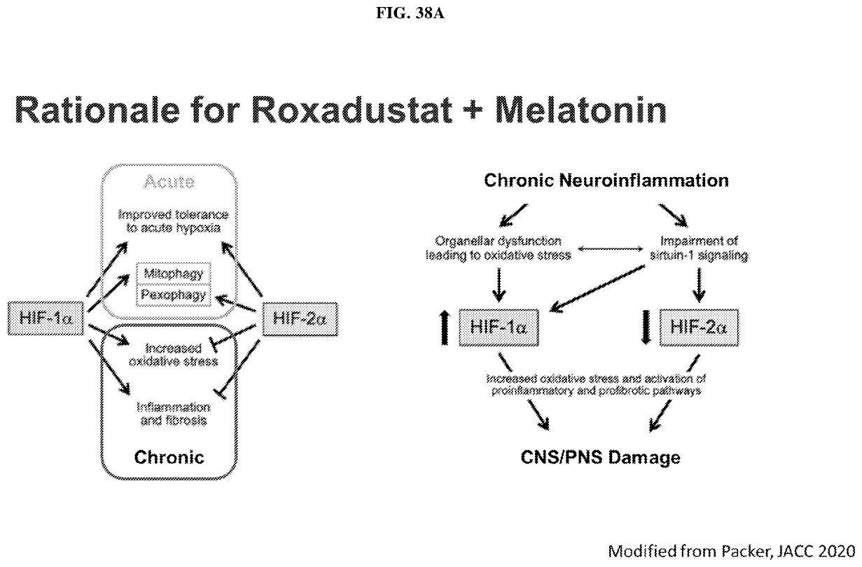

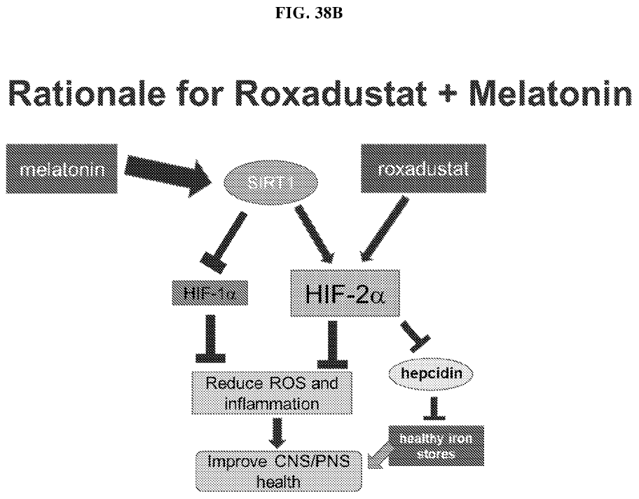

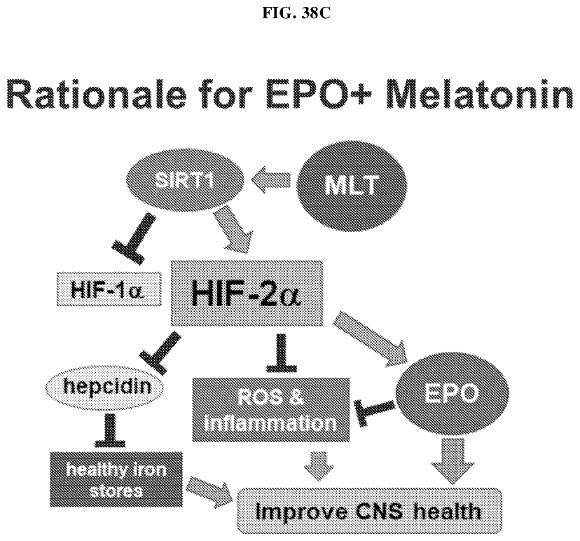

After establishing the PCL model, we then rationally designed a therapy to address the pathophysiology. Among others, we propose a combined use of melatonin and prolyl hydroxylase domain (PHD) inhibitor for the treatment of Post-COVID-Like (PCL) syndrome (FIG. 38A-38C). For example, melatonin has receptor-independent anti-oxidant effects, so it has been used as adjunct with chemotherapy to increase efficacy. Melatonin (MLT) may be used 20 mg/kg/dose (safe human range is up to 200 mg/kg/dose). Further, PHD inhibitor includes about 10 drugs in various stages in clinical trials pathway such as adaptaquin, roxadustat (FG-4592), The PHD inhibtiors may be effective in multiple organs such as renal, cardiac, atherosclerosis, obesity, pulmonary, mitochondria and ferroptosis, retinopathy of prematurity, bone healing, skeletal muscle, inflammatory bowel disease, nervous system (in vivo): spinal cord injury, depression, Parkinson's disease.

Particularly, FIG. 38B shows rational mechanistically defined rox+MLT therapy. Melatonin (MLT) upregulates SIRT1 and thus favorably shifts the HIF1a/HIF2a balance towards HIF2a. Via proly hydroxylase domain (PHD) inhibition, Roxadustat (ROX) also increases HIF2a. Together, MLT plus ROX reduce oxidative stress (ROS—reactive oxygen species) and inflammation following SARS-CoV-2 illness. Notably, HIF2a blocks hepcidin and promotes health Iron stores. Via complementary mechanisms, this cocktail therapy may improve central nervous system (CNS) and peripheral nervous system (PNS) function.

FIG. 38C shows rational mechanistically defined EPO+MLT therapy. Melatonin (MLT) upregulates SIRT1 and thus favorably shifts the HIF1a/HIF2a balance towards HIF2a. HIF2a increases EPO production which improves neural cell health and development. Together, MLT plus EPO reduce oxidative stress (ROS—reactive oxygen species) and inflammation following SARS-CoV-2 illness. Notably, HIF2a blocks hepcidin and promotes health Iron stores. Via complementary mechanisms, this cocktail therapy may improve central nervous system (CNS) and peripheral nervous system (PNS) function.

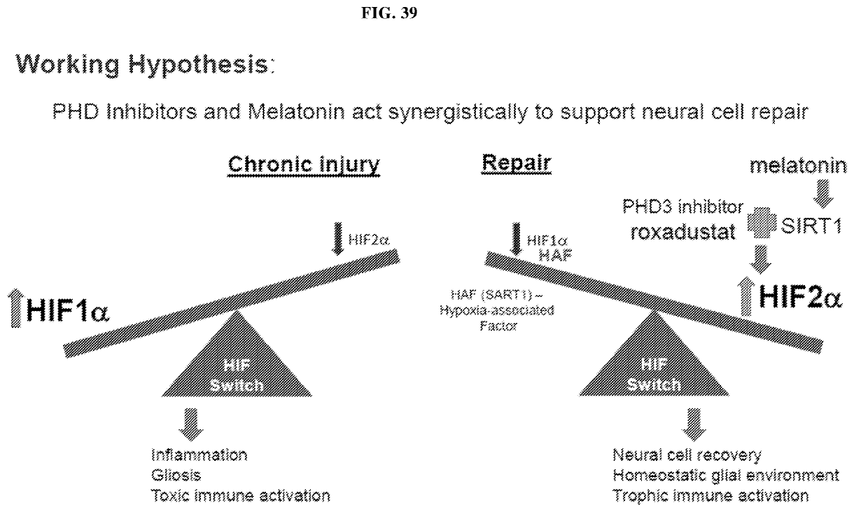

FIG. 39 shows an exemplary hypothesis that PHD inhibitors and melatonin act synergistically to support neural cell repair. In synergistic neural cell repair, melatonin (MLT) upregulates SIRT 1 and thus favorably shifts the HIF1a/HIF2a balance towards HIF2a. Via prolyl hydroxylase domain (PHD) inhibition, Roxadustat (ROX) also increases HIF2α. Together, MLT plus ROX reduce oxidative stress (ROS—reactive oxygen species) and inflammation following SARS-CoV-2 illness. Via complementary mechanisms, this cocktail therapy may improve central nervous system (CNS) and peripheral nervous system (PNS) function. Chronic injury in Post COVID syndrome is defined by too much HIF1a and inflammation, gliosis and toxic immune activation. Our therapy, shifts this balance to repair by increasing HIF2a and facilitating neural cell recovery, trophic immune activation and promoting a homeostatic glial environment.

As of preclinical Post-COVID-Like Syndrome treatment, the following design is proposed
Randomized to vehicle or treatment and observers blinded
Melatonin (MLT) 20 mg/kg/dose×10 doses
Prolyl hydroxylase domain inhibitor
   Adaptaquin (ADQ) 30 mg/kg/dose×4 doses
   Roxadustat cohort 1: 10 mg/kg/dose×10 doses
   Roxadustat cohorts 2 and 3: 15 mg/kg/dose×10 doses
   Results thus far: We found that 15 mg/kg/dose did not provide significant additional benefit over 10 mg/kg/dose
   Adaptaquin+MLT: pain phenotype, PBMCs, histology, biochem
   Roxadustat cohort 1: pain phenotype, cognition, histology, biochemical analysis of brain, immune cell characterization with multiparametric flow cytometry, advanced magnetic resonance imaging including diffusion tensor imaging and functional resting-state MRI

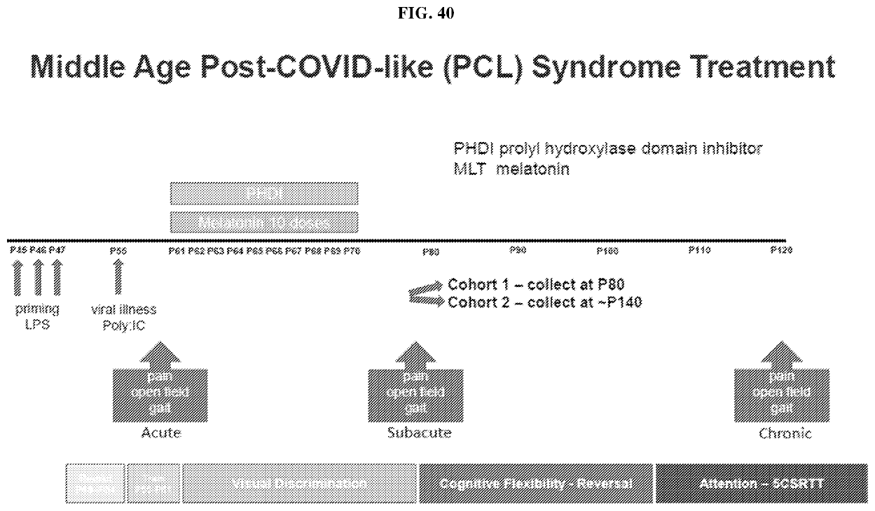

FIG. 40 shows an exemplary model treatment of middle age Post-COVID-like (PCL) Syndrome Treatment. For experimental design and development of pel syndrome with cognitive assessment, LPS (3 mg/kg ip) is given to prime the immune system of rats on postnatal (P) 45, 46 and P47. Subsequently, Poly IC (5 mg/kg ip) is given on P55 to mimic a viral exposure. The response to these stimuli are studied in the following acute, subacute and chronic timepoints. Pain and Activity in an Open Field are outcome measures we use routinely to track the development of the PCL syndrome. Similarly, Touchscreen cognitive assessment occurs simultaneously throughout the Acute, SubAcute and Chronic Phases. Using a touchscreen assessment platform, we first assess visual discrimination. We then assess cognitive flexibility through a test of reversal learning, and then a test of attention called 5 choice serial reaction (5CSRTT).

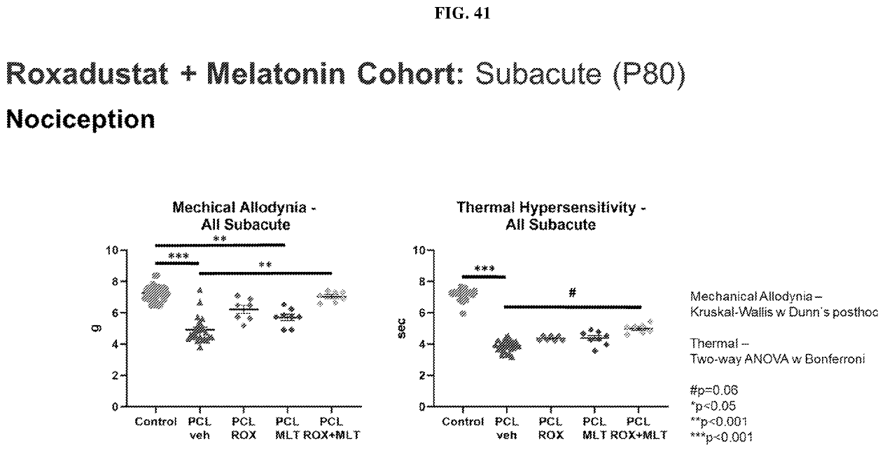

### Example 4.1. Subacute Treatment with ROX+MLT Attenuates Pain in a Post-COVID Like (PCL) Model of Chronic Neurological Sequelae (FIG. 41)

After priming the immune system with systemic lipopolysaccharide (LPS) on postnatal day 45-47, rats receive the viral mimetic poly(I:C) on P55. Rats with PCL treated with vehicle exhibit mechanical allodynia and thermal hypersensitivity. Notably, treatment with ROX+MLT, but neither ROX nor MLT alone, mitigates mechanical allodynia and thermal sensitivity (Two-Way ANOVA with Bonferroni correction, *p<0.05; **p<0.01; ***p<0.001; PIC=PolyIC; PCL=Post-COVID like which is LPS+PIC; MLT=Melatonin; ROX=Roxadustat).

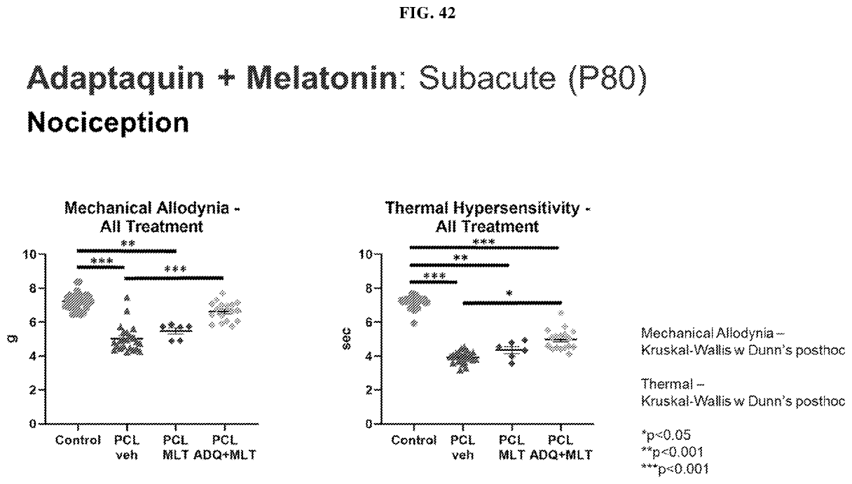

### Example 4.2. Subacute Treatment with Adaptaquin+MLT Attenuates Pain in a Post-COVID Like (PCL) Model of Chronic Neurological Sequelae (FIG. 42)

After priming the immune system with systemic lipopolysaccharide (LPS) on postnatal day 45-47, rats receive the viral mimetic poly(I:C) on P55. Rats with PCL treated with vehicle exhibit mechanical allodynia and thermal hypersensitivity. Treatment with the PDHI Adaptaquin+MLT alleviates thermal hypersensitivity and allodynia (Two-Way ANOVA with Bonferroni correction, *p<0.05; **p<0.01; ***p<0.001; PIC=PolyIC; PCL=Post-COVID like which is LPS+PIC; MLT=Melatonin; ADQ=Adaptaquin).

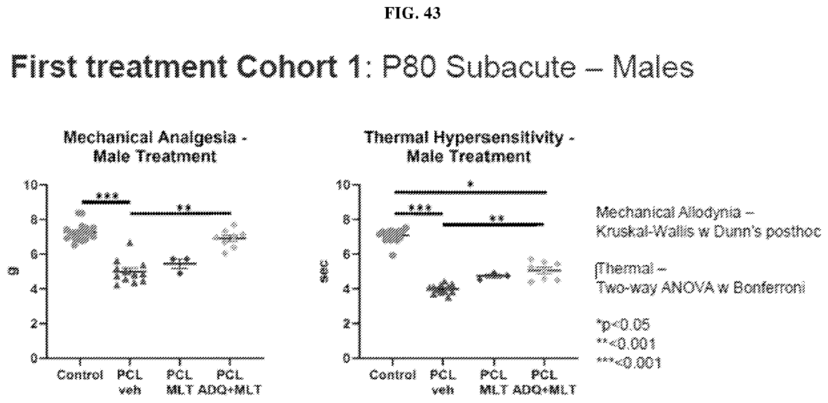

FIG. 43 shows that subacute treatment with adaptaquin+MLT attenuates pain in male animals in a Post-Covid Like (PCL) model of chronic neurological sequelae. After priming the immune system with systemic lipopolysaccharide (LPS) on postnatal day 45-47, rats receive the viral mimetic poly(I:C) on P55. Male rats with PCL treated with vehicle exhibit mechanical allodynia and thermal hypersensitivity. Treatment with the PDHI Adaptaquin+MLT alleviates thermal hypersensitivity and allodynia in male rats (Two-Way ANOVA with Bonferroni correction, *p<0.05; **p<0.01; ***p<0.001; PIC=PolyIC; PCL=Post-COVID like which is LPS+PIC; MLT=Melatonin; ADQ=Adaptaquin).

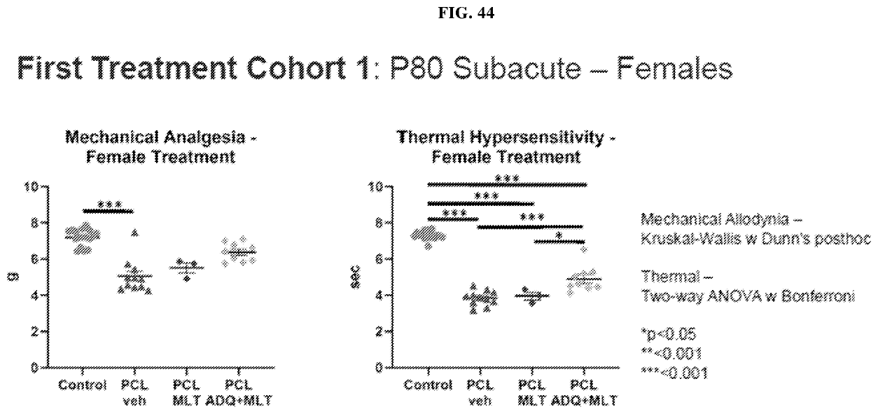

FIG. 44 shows that subacute treatment with adaptaquin+mlt attenuates pain in a Post-COVID like (PCL) model of chronic neurological sequelae. After priming the immune system with systemic lipopolysaccharide (LPS) on postnatal day 45-47, rats receive the viral mimetic poly(I:C) on P55. Female rats with PCL treated with vehicle exhibit mechanical allodynia and thermal hypersensitivity. Treatment with the PDHI Adaptaquin+MLT alleviates thermal hypersensitivity in female rats but does not improve allodynia (Two-Way ANOVA with Bonferroni correction, *p<0.05; **p<0.01; ***p<0.001; PIC=PolyIC; PCL=Post-COVID like which is LPS+PIC; MLT=Melatonin; ADQ=Adaptaquin).

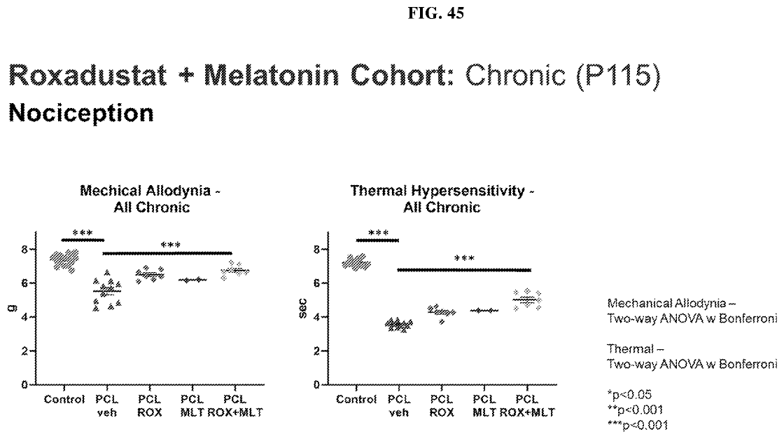

### Example 4.3. Treatment with ROX+MLT Attenuates Chronic Pain in a Post-COVID Like (PCL) Model (FIG. 45)

After priming the immune system with systemic lipopolysaccharide (LPS) on postnatal day 45-47, rats receive the viral mimetic poly(I:C) on P55. Rats with PCL treated with vehicle exhibit chronic mechanical allodynia and thermal hypersensitivity that lasts greater than 2 months. Notably, treatment with ROX+MLT, but neither ROX nor MLT alone, mitigates mechanical allodynia and thermal sensitivity (Two-Way ANOVA with Bonferroni correction, *p<0.05; **p<0.01; ***p<0.001; PIC=PolyIC; PCL=Post-COVID like which is LPS+PIC; MLT=Melatonin; ROX=Roxadustat).

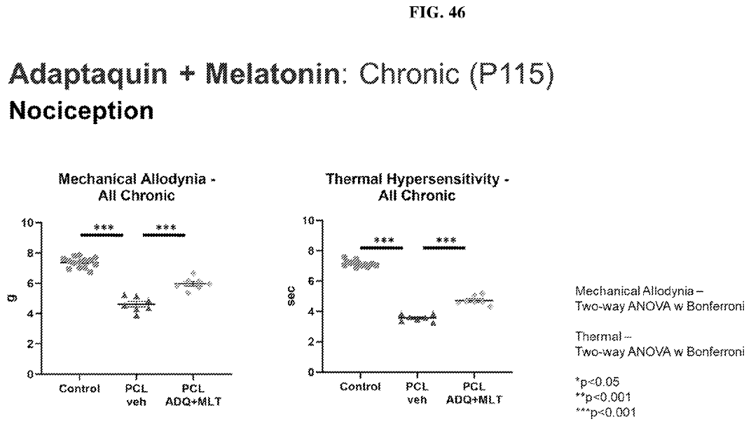

### Example 4.4. Treatment with ADQ+MLT Attenuates Chronic Pain in a Post-COVID Like (PCL) Model (FIG. 46)

After priming the immune system with systemic lipopolysaccharide (LPS) on postnatal day 45-47, rats receive the viral mimetic poly(I:C) on P55. Rats with PCL treated with vehicle exhibit chronic mechanical allodynia and thermal hypersensitivity that lasts greater than 2 months. Notably, treatment with ADQ+MLT mitigates mechanical allodynia and thermal sensitivity (Two-Way ANOVA with Bonferroni correction, *p<0.05; **p<0.01; ***p<0.001; PIC=PolyIC; PCL=Post-COVID like which is LPS+PIC; MLT=Melatonin; ADQ=Adaptaquin).

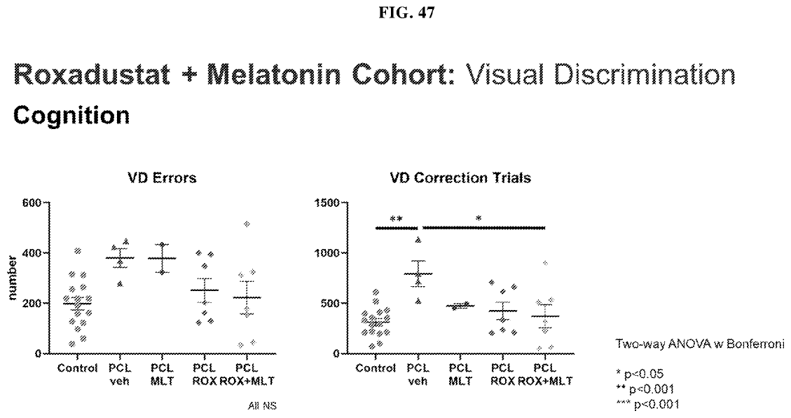

### Example 4.5. Subacute Treatment with ROX+MLT Alleviates Deficits of Cognition in a Post-COVID Like (PCL) Model of Chronic Neurological Sequelae (FIG. 47)

After priming the immune system with systemic lipopolysaccharide (LPS) on postnatal day 45-47, rats receive the viral mimetic poly(I:C) on P55. Rats with PCL treated with vehicle exhibit deficits in cognition. Notably, treatment with ROX+MLT, but neither ROX nor MLT alone, improved cognition on a touchscreen assay (Two-Way ANOVA with Bonferroni correction, *p<0.05; **p<0.01; ***p<0.001; PIC=PolyIC; PCL=Post-COVID like which is LPS+PIC; MLT=Melatonin; ROX=Roxadustat).

The above results show that clinically-relevant preclinical models, functional outcomes, repurposing drugs. Also indicated are recent progress with HIF1a/2a imbalance and approval of PHD3 inhibitor Roxadustat, which may be important for overwhelming urgency and impact of long COVID.

The goal may include safety test of a combination of PHD inhibitor (e.g., roxadustat) and melatonin in preclinical SARS-CoV-2 model and mechanistic studies. Therefore, implications for other chronic neurological symptoms for similar insults such as after sepsis, other critical illness may be investigated.

### Example 5: Treatment Protocol for a Subject Suffering from Prenatal Opioid Exposure

A pregnant female patient is selected for treatment suffering from or susceptible to prenatal opioid exposure, or having brain injury associated with in utero and perinatal opioid exposure.

A pharmaceutical composition including a melatonin agent and an erythropoietin compound is prepared in a sterile aqueous solution is administered by intravenous injection. The dosing regimen may include 400 U/kg/day of EPO and/or 20 mg/kg/day melatonin (MLT).

### Example 6: Treatment Protocol for a Subject Suffering Traumatic Brain Injury

An adult human male patient is selected for treatment after suffering severe concussion. Treatment is initiated as soon as possible after the head trauma, preferably with 0.25, 0.5, 1, 2, 3, 4, 5, 6, 7, 8, 9, 10, 12, 24, 36, 48, 72 or 96 or hours or less after the head trauma.

The subject is treated every 8 hours with the following doses of roxadustat and melatonin: 10 mg/kg/day of roxadustat (ROX) and/or 20 mg/kg/day melatonin (MLT).

### Example 7: Treatment Protocol for a Subject Having a COVID-19 Infection

A human patient is selected for treatment after testing positive for a COVID-19 infection, and surviving the acute illness. The treatment regimen as proposed will be administered in the subacute phase of illness to enhance neurological recovery and minimize the risk of long term neurological sequelae.

The subject is treated with a regimen of induction followed by maintenance treatment. The induction is treatment every 24 hours for 10 days with the following dosages of roxadustat and melatonin: 5 mg/kg/day of roxadustat (ROX) daily and 20 mg/kg/day melatonin (MLT) qhs. Maintenance regimen is Roxadustat 3 mg/kg/dose and 20 mg/kg/dose melatonin every other day (MWF) every other week.

### Example 8

The subject is an infant admitted to the neonatal intensive care unit (NICU) with confirmed opioid exposure. The following treatment protocol is administered to the subject:
EPO=IV or SC plus MLT=oral
rhEPO will be 1000 U/kg/dose qod×10 doses then 400 U/kg/dose q MWF until 33w6/7d MLT will be 20 mg/kg qd enteral until 33w6/7d

### Example 9

For attenuation of cognitive defects and white matter injury associated with perinatal opioid exposure the following treatment protocol can be used.
Children with Opioid exposure in utero and NOWS and young adults with SUD
rhEPO will be 1000 U/kg dose IV/SC q wk×6 weeks OR
Melatonin will be 20 mg qhs enteral×6 wks

### Example 10

Infant with moderate-severe intraventricular hemorrhage and concern for post-hemorrhagic hydrocephalus of prematurity. The following treatment protocol can be utilized:

Subjects who are born <32 w PMA and who have severe IVH on HUS within 5 days. Inclusion Criteria are 1) born at <32 w PMA, 2) severe IVH, defined as at least unilateral grade III⁶, 3) expected to survive at least 3 days, 4) absence of a congenital anomaly of metabolic or genetic disorder with expected survival less than term equivalent, 5) approval of the primary neonatologist, 6) arterial or venous access, 7) appropriate caregiver to provide informed consent. Exclusion criteria: 1) life expectancy <3 days, 2) severe congenital anomaly or genetic disorder with life expectancy <40 w PMA 3) severe hematologic crisis such as disseminated intravascular coagulation 4) hydrops fetalis, 5) polycythemia (hematocrit <65%), 6) hypertension for age requiring medication, 7) clinical concern or diagnosis of toxoplasmosis, cytomegalovirus rubella or syphilis infection, 8) no appropriate person available or willing to provide informed consent.

Dosage: rhEPO will be 1000 U/kg/dose intravenous (IV) qod for 10 doses followed by 400 U/kg/dose subcutaneous (sc) qMWF until the subject is 33w6/7d. Placebo for rhEPO will be the same volume of sterile saline prepared for injection.

### Example 11: Covid-19 Treatment

The following treatment protocol can be used for a patient with post-acute COVID or similar adults with chronic neuroinflammatory disorders who are preparing for inpatient rehabilitation after the acute illness begins to subside.

Induction Roxadustat (70 or 100 mg tablets) 5 mg/kg/dose daily and melatonin 20 mg/kg/dose qhs for 10 days followed by maintenance regimen of Roxadustat 3 mg/kg/dose and Melatonin 20 mg/kg/dose evry other day (MWF) given every other week.

2 mg/kg/dose qMWF×3 weeks plus melatonin 20 mg qhs for 3 weeks.

### Example 12—Roxadustat+Melatonin: 5 Choice Serial Reaction Time Test

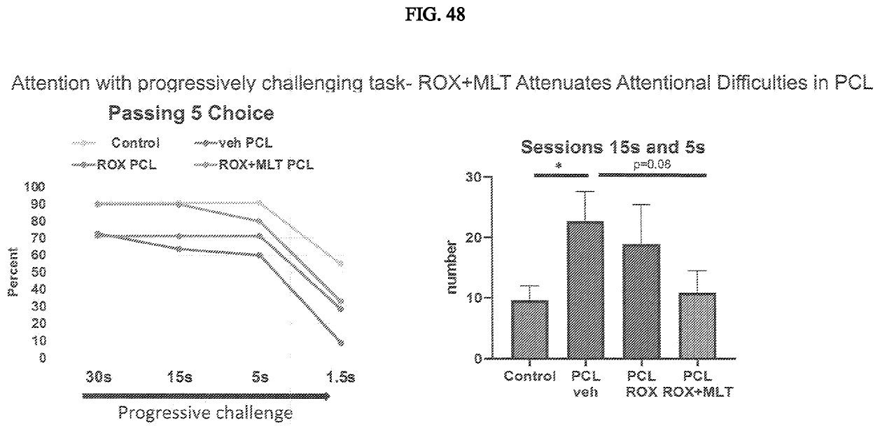

The 5-choice serial reaction time test (5CSRTT) is used in both humans and rodents to test the attentional component of cognition. During the test, the subject completes a task under progressively shorter testing intervals. In the rat, the testing intervals decrease from 30 sec to 15 sec to 5 sec to 1.5 sec. Results are shown in FIG. 48. As shown in the in the chart on the left of FIG. 48, controls have a high pass rate until the test interval decreases to 1.5 sec. By contrast, rats with long COVID treated with vehicle (PCL-veh) had reduced performance at all intervals, which was most evident at 1.5 sec. Treatment with the cocktail of Roxadustat (ROX) and Melatonin (MLT) improves performance. In the chart on the right of FIG. 48, the total number of sessions to pass the 15 sec and 5 sec intervals is significantly more in the PCL-veh compared to controls. Treatment with the cocktail of Roxadustat (ROX) and Melatonin (MLT) almost normalizes the session number, whereas ROX alone is not significantly improve. Kruskal-Wallis with Dunn's, * p<0.05.

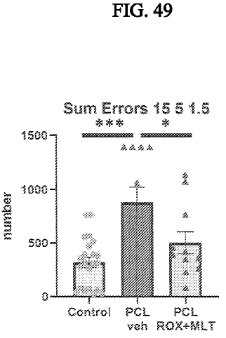

In the results shown in FIG. 49, during the test, the subject completes a task under progressively shorter testing intervals. In the rat, the testing intervals decrease from 30 sec to 15 sec to 5 sec to 1.5 sec. Rats with long covid treated with vehicle (PCL-VEH) make significantly more errors from tasks with a shorter time to complete (15 sec, 5 see, 1.5 sec) during the chronic phase of injury compared to controls. On the other hand, induction Treatment with ROX+MLT in the subacute phase of injury provides sustained protection. Specifically, the number of Errors is significantly lower with ROX+MLT treatment. *p<0.05; ***p<0.001.

### Example 13—Roxadustat+Melatonin Induction and Maintenance Regimen

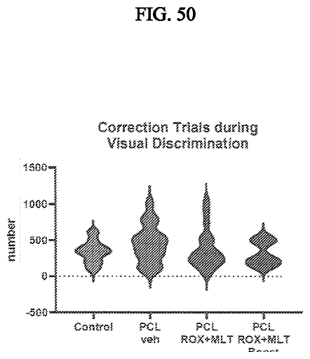

It was found that ROX+MLT treatment can improve neurological recovery in long covid through neuro-immunomodulation. To further supplement the ongoing reduction in inflammation, a maintenance regimen of ROX+MLT was added to the initial treatment course. This is termed the ROX+MLT Boost regimen. FIG. 50 shows results that the ROX+MLT Boost regimen provides additional improvement in performance on a test of Visual Discrimination. When a subject makes an incorrect choice, a correction trial is given. Similar to controls, animals with long covid treated with ROX+MLT in the subacute phase for 10 days and given a maintenance boost every ˜15 days, require less correction trials. These data are consistent with improved cognitive performance compared to animals with long covid treated with vehicle or exclusively a sub acute ROX+MLT dosing regimen.

### Example 14—Diffusion Tensor Imaging-ROX+MLT Attenuates White Matter Loss Secondary to PCL

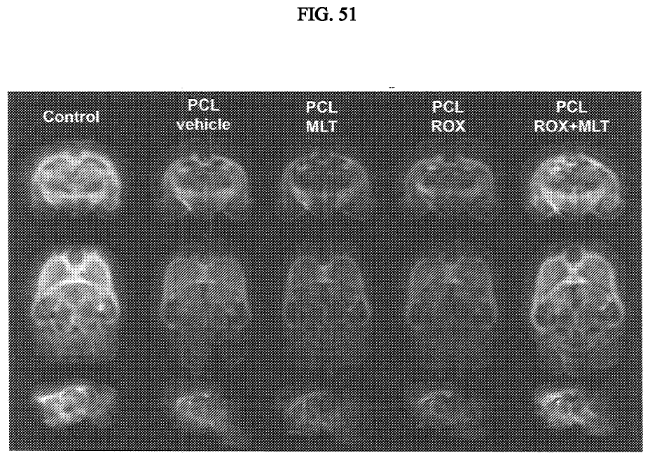

Data shows that part of the neurological damage from PCL is due to white matter injury. Diffusion Tensor Imaging was performed on an 11.7T Bruker MRI scanner. These images were processed to assign directional colors (yellow transverse, blue vertical, and green orthogonal to the plane). As shown in FIG. 51, when more injury is present, the microstructural integrity is altered and the colors are muted, as shown in the PCL vehicle, PCL MLT monotherapy, and PCL ROX monotherapy, compared to the Control and the ROX+MLT-treated PCL.

### Example 15

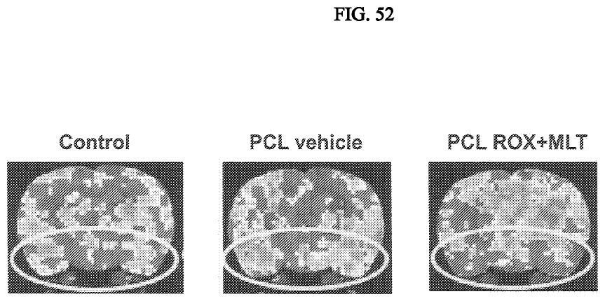

To further clarify the changes in brain function associated with long COVID, in vivo functional MRI (fMRI) was performed Functional MRI shows increased activation in the entorhinal cortex in vehicle-treated PCL rats. Treatment with ROX+MLT normalizes the brain activation. Specifically, PCL aberrantly increases functional activation and the mean amplitude of low frequency fluctuations in the entorhinal cortex. ROX+MLT restores normal functional activation and restores normal neural activity. Results are shown in FIG. 52 which depicts functional activation in fMRI in this PCL model.

### Example 16—Erythropoietin (EPO) Plus Melatonin (MLT) Attenuates Posthemorrhagic Hydrocephalus (PHH)-Induced Cognitive Deficits of Visual Discrimination as Assessed on a Touchscreen Platform

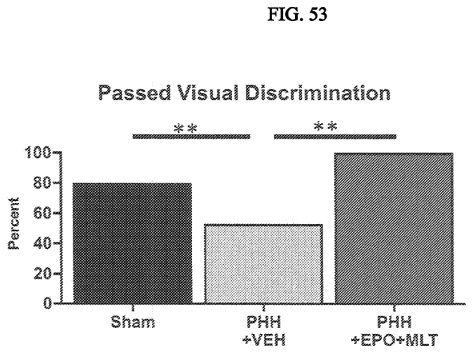

People with posthemorrhagic hydrocephalus (PHH) have cognitive problems. Visual discrimination tests spatial memory. Vehicle-treated rats with PHH show much more difficulty passing visual discrimination than controls, or PHH rats treated with a regimen of Erythropoietin plus Melatonin. **p<0.01). Results are depicted in FIG. 53.

### Example 17

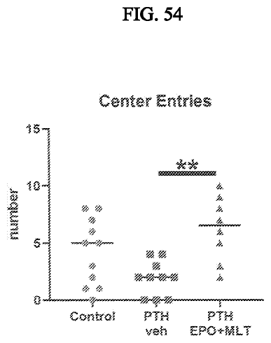

Erythropoietin (EPO)+Melatonin (MLT) Attenuates Deficits in Open Field Behavior, including center exploration, in animals with post-traumatic hydrocephalus (PTH). Rats with post-traumatic hydrocephalus (PTH) were tested in an open field to assay exploration. A circular arena is divided into three concentric rings: wall, neutral zone and center. The activity of a rat is videotaped over 15 minutes and analyzed in three 5-minute intervals. Control rats explore the arena and occasionally enter the center ring. Vehicle-treated rats with PTH demonstrate fewer center entries, and thus less exploration than control rats. By contrast, PTH rats treated with erythropoietin (EPO) and melatonin (MLT) exhibit a more normal pattern of behavior. These data are concomitant with attenuation of deficits of inhibition. **p<0.01. Results are depicted in FIG. 54.

### Example 18

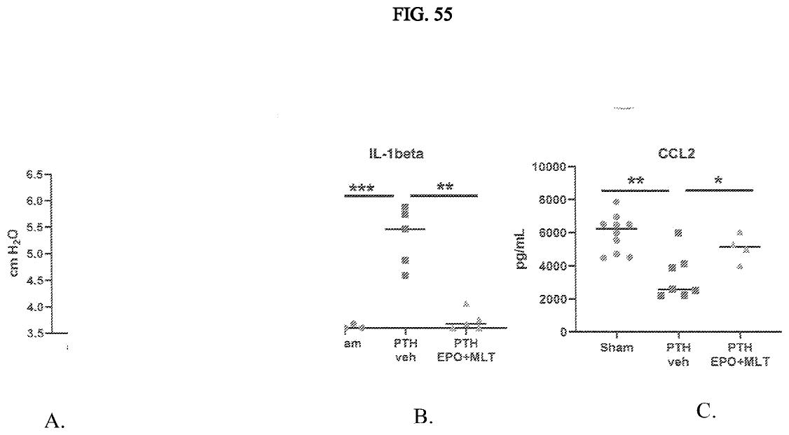

Erythropoietin (EPO)+Melatonin (MLT) Treatment Normalizes Intracranial Pressure and Blood Inflammatory Biomarkers after post-traumatic hydrocephalus (PTH). Systemic administration of EPO+MLT after traumatic brain injury prevents pathological increases in intracranial pressure (opening pressure) consistent with PTH. Similarly, EPO+MLT normalizes levels of the pro-inflammatory cytokine interleukin 1beta and CCL2, a chemokine central to monocyte trafficking. These data are consistent with normalized blood levels of detrimental inflammatory proteins in subjects with hydrocephalus treated with EPO+MLT *p<0.05,**p<0.01;***p<0.001. FIG. 55 (includes FIGS. 55A-C) depicts results showing EPO+MLT Treatment Normalizes Post-traumatic Hydrocephalus.

### Example 19

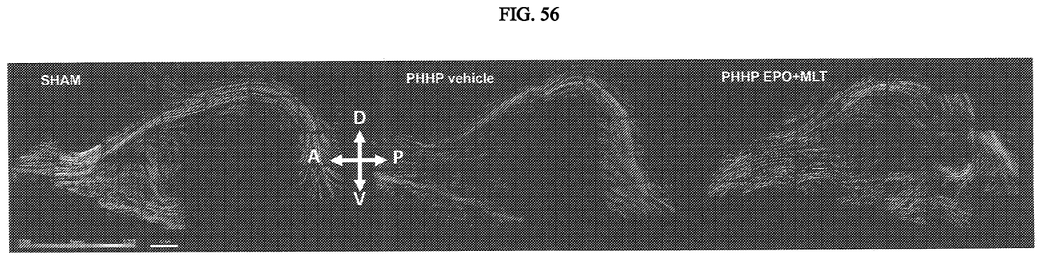

FIG. 56 depicts results showing Erythropoietin (EPO)+melatonin (MLT) treatment resolve deficits in cilia function and cerebral spinal fluid (CSF) flow in posthemorrhagic hydrocephalus of prematurity (PHHP). FIG. 56 is a live cell ependymal cilia video microscopy of propelled fluorescent beads on lateral ependymal wall of PHHP rats reflect motile cilia function (red/brighter colors represent more flow and faster ciliary beating). Bead speed and angle deviation are calculated. Rats with hydrocephalus (PHHP-VEH) have significant cilia injury. This is repaired by EPO+MLT. Bar=400 mm.

### Example 20

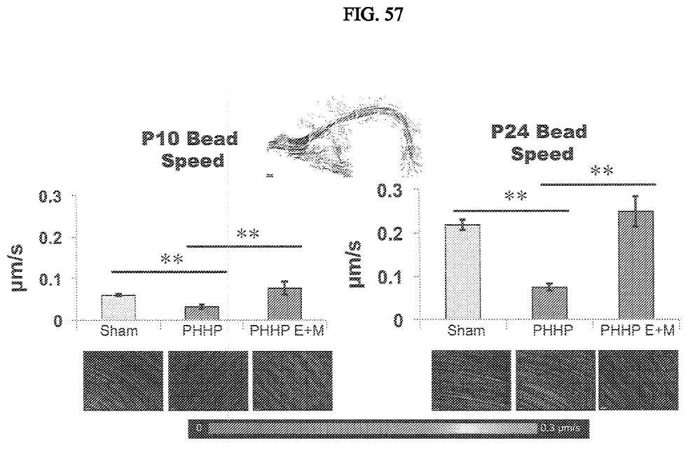

FIG. 57 depicts results showing EPO+MLT Rescues Ependymal Motile Cilia Impairment. Florescent bead speed was used to quantify ependymal motile cilia function. Motile cilia mature in rats from postnatal day 3 (P3) to P24. In Sham controls, bead speed increases 4-fold from −0.05 mm/sec at P10 to ˜0.2 mm/sec at P24. By contrast, in vehicle-treated PHHP rats at P10, bead speed is limited compared to shams, and normalized by EPO+MLT treatment. Moreover, in vehicle-treated PHHP rats, motile cilia fail to mature by P24, whereas EPO+MLT normalizes motile cilia function at P24. **p<−0.01.

### Example 21

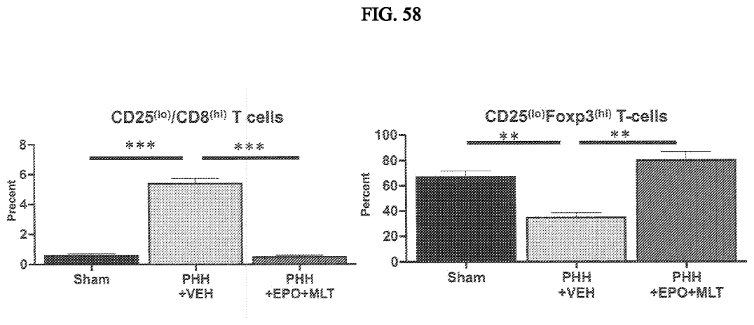

FIG. 58 shows Erythropoietin (EPO)+Melatonin (MLT) Mitigates Posthemorrhagic hydrocephalus (PHHP)-Driven Increases in Activated T-Cells and Detrimental Decreases in Anti-Inflammatory T-Cells. These data are consistent with treatment beneficially restoring immune system function and cell populations after hydrocephalus. **p<0.01; ***p<0.001.

### Example 22

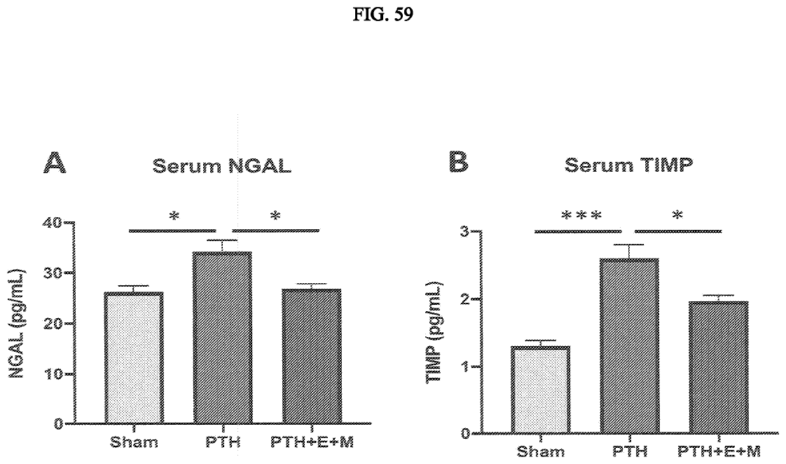

Combination therapy with erythropoietin (EPO)+melatonin (MLT) improves neural cell health biomarkers following post-traumatic hydrocephalus (PTH). In FIG. 59A, pathological increases of Lipocalin 2 (NGAL) are reversed by EPO+MLT treatment in PTH animals. In FIG. 59B, PTH increases TIMP (tissue inhibitors of metalloproteinases) levels in the serum, a marker consistent with impaired neural cell survival. EPO+MLT attenuates increases in TIMP restoring homeostasis and decreasing neural injury. These data are consistent with a robust effect on EPO+MLT on neural health. (*p<0.05, ***p<0.001).

### Example 23—EPO+MLT Treatment Improves White Matter Integrity in Post-Traumatic Hydrocephalus (PTH)

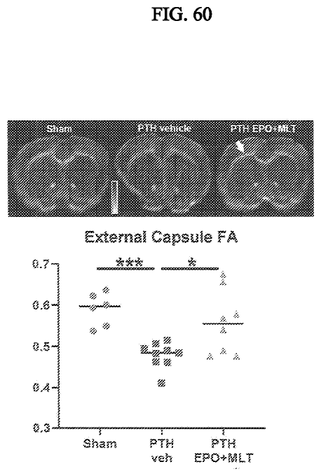

Erythropoietin (EPO)+melatonin (MLT) Normalizes White Matter Microstructure in Rats with post-traumatic hydrocephalus (PTH). Systemic administration of EPO+MLT after traumatic brain injury leading to hydrocephalus normalizes structural coherence and fractional anisotropy (FA) in major white matter tracts including the external capsule. These data are consistent with EPO+MLT induced repair of injured white matter and improved brain microstructure. *p<0.05; ***p<0.001). Results are shown in FIG. 60.

### Example 24—EPO+MLT Treatment Improves White Matter Integrity in Post-Traumatic Hydrocephalus (PTH)

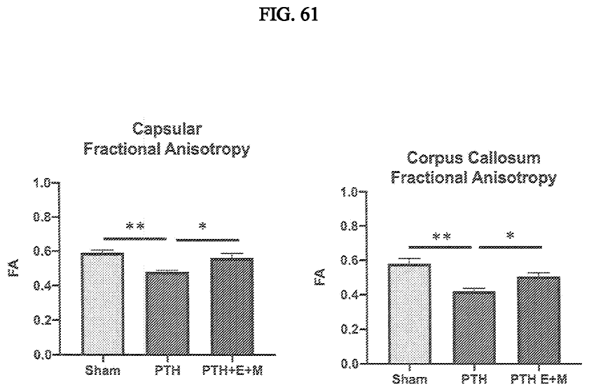

Fractional anisotropy (FA) loss induced by post-traumatic hydrocephalus (PTH) was reversed by treatment with erythropoietin (E) and melatonin (M). Quantitative analyses demonstrate PTH animals have significantly reduced FA in the corpus callosum, and external capsule compared to sham and E+M treated PTH rats in adulthood. (*p<0.05, **p<0.01). These data are consistent with neurorepair induced by E+M. Results are shown in FIG. 61.

### Example 25

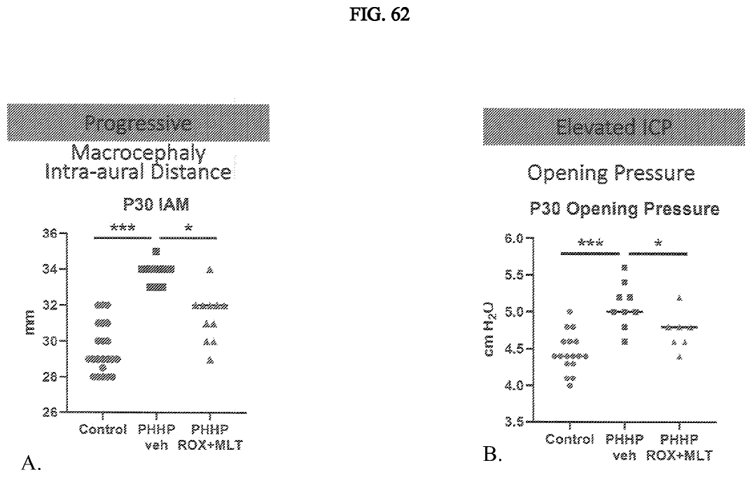

FIG. 62 (includes FIGS. 62A and 62B) depicts results showing ROX+MLT Attenuates Macrocephaly and Increased Intracranial Pressure (ICP) in Existing Posthemorrhagic Hydrocephalus of Prematurity (PHHP). Roxadustat (ROX) plus Melatonin (MLT) treatment of existing PHHP during infancy from postnatal day 10 (P10) to P19 normalizes macrocephaly measured with Intra-aural Measurement (IAM), a surrogate for head circumference, compared to vehicle-treated rats with PHHP. Likewise, ROX+MLT treats elevated intracranial pressure measured by opening pressure at the cisterna magna.

### Example 26

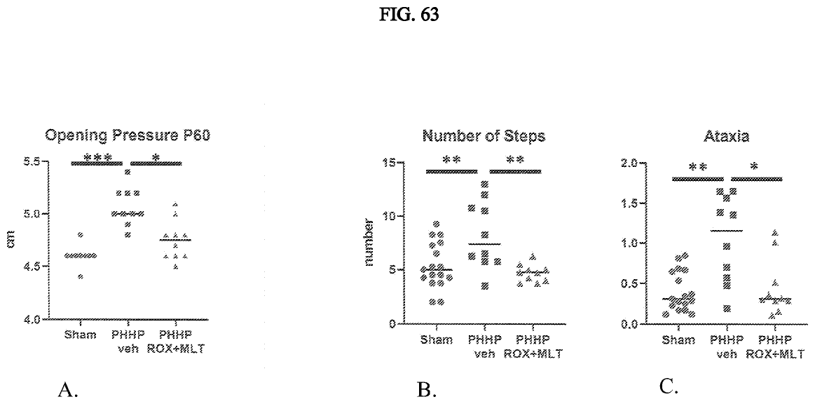

Treatment with Roxadustat (ROX) plus Melatonin (MLT) during childhood equivalent age (postnatal day 21 (P21) to P30) normalizes intracranial pressure (opening pressure) in adult rats with PHHP at P60. Rats with PHHP also exhibit an abnormal gait that mimics the spastic gait of cerebral palsy, with more frequent steps and ataxia. Childhood treatment with ROX+MLT normalizes gait. *p<0.05, **p<0.01, ***p<0.001. Results are shown in FIG. 63 (includes FIGS. 63A-63C) depicts results showing ROX+MLT Attenuates Increased Intracranial Pressure and Gait in Existing Posthemorrhagic Hydrocephalus of Prematurity (PHHP).

### Example 27

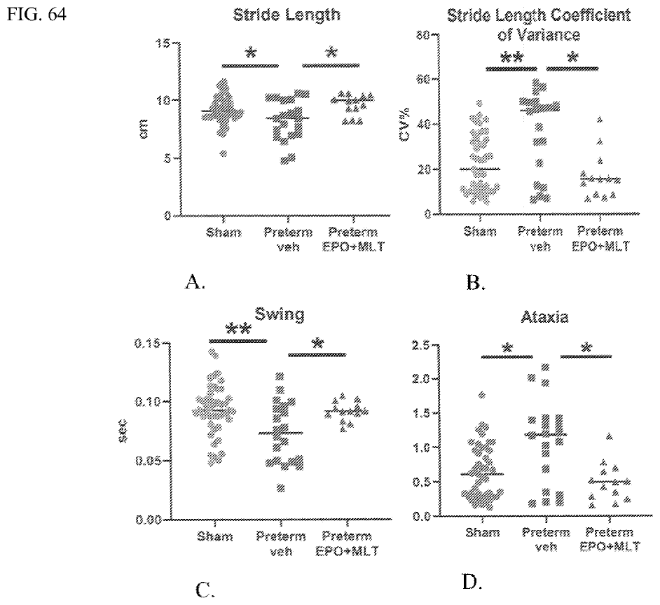

FIG. 64 (includes FIGS. 64A-64D) depicts results showing EPO+MLT Normalizes Gait Metrics in a Model of Cerebral Palsy Secondary to Preterm Brain Injury. Rats with preterm brain injury have shorter stride length, more variation in stride length, and shorter swing phase than sham rats. Rats with preterm brain injury also exhibit more ataxia. All of these metrics are restored with EPO+MLT treatment given in infancy from postnatal day 15 (P15) through P20. Gait was assessed at P30 using digital treadmill analyses. *p<0.05, **p<0.01.

### Example 28

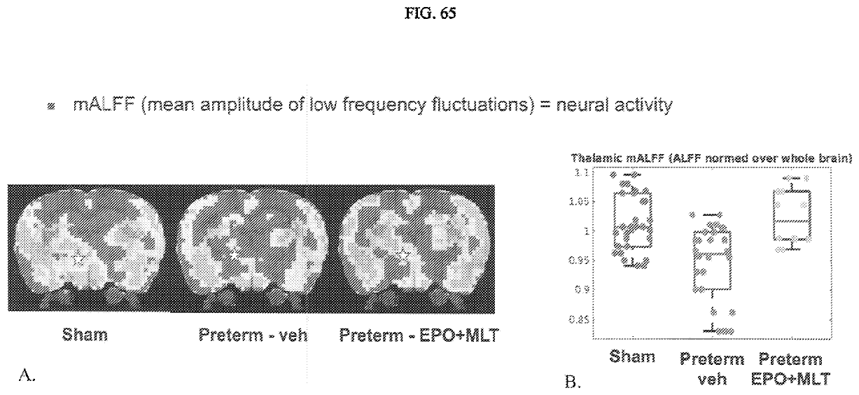

FIG. 65 (includes FIGS. 65A-65B) depicts results showing neonatal EPO+MLT administered from postnatal day 1 (P1) through P10 normalizes functional activation in the thalamus (white star) in adult rats with cerebral palsy secondary to chorioamnionitis.

### Example 29—Roxadustat (ROX) and Melatonin (MLT) Treatment Attenuates Nociception Induced by Mechanical and Thermal Stimuli in Adult Rats with Preterm Brain Injury

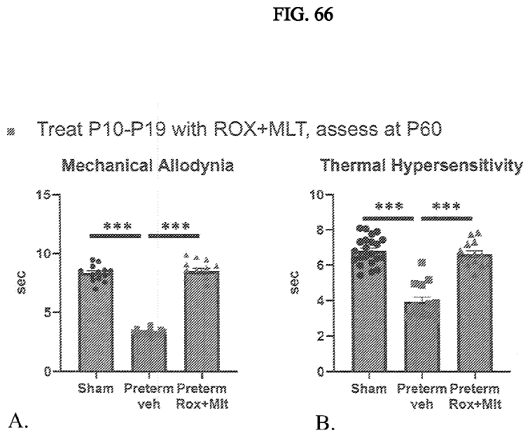

After prenatal injury to mimic CNS injury from very preterm birth, injured rats were randomly allocated to treatment or vehicle during infancy from postnatal day 10 (P10) to P19 with ROX+MLT, and then the pain phenotype was evaluated in young adult rats at P60. Mechanical allodynia was tested using von Frey filaments with the up-down method, and thermal sensitivity was measured with tail immersion at 48° C. Treatment with ROX+MLT during infancy prevents emergence of the pain phenotype after preterm CNS injury. (***p<0.001). Results are shown in FIG. 66 (includes FIGS. 66A-66B).

### Example 30—EPO+MLT Mitigates Deficits in Inhibition Observed on an Open Field Test in Adult Animals Prenatally Exposed to Methadone

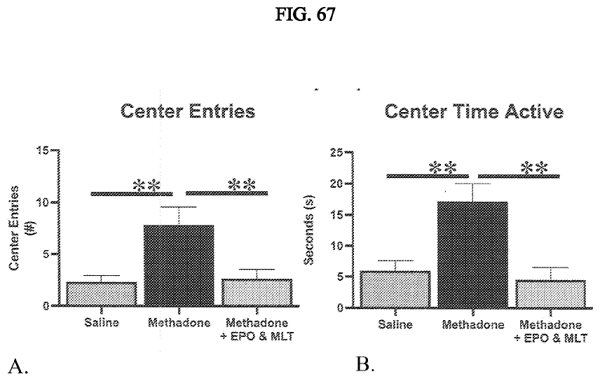

To mimic perinatal opioid exposure (POE), infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) or vehicle from P1-P10. For Open Field testing at P35, an individual rat is placed in a circular arena is divided into three concentric rings: wall, neutral zone and center. The activity of the rat is videotaped over 15 minutes and analyzed in three 5-minute intervals. Control rats occasionally enter the center area but spend most of the time in the wall region. Rats exposed to POE treated with vehicle enter the center area more often and spend more time active in the center compared to saline controls, or POE-exposed rats treated with EPO+MLT. These data are consistent with EPO+MLT restoring behavioral inhibition after POE. (**p<0.01). Results are shown in FIG. 67 (includes FIGS. 67A-67B).

### Example 31—EPO+MLT Attenuates Hyperactivity in Adult Rats Exposed to Methadone in Utero

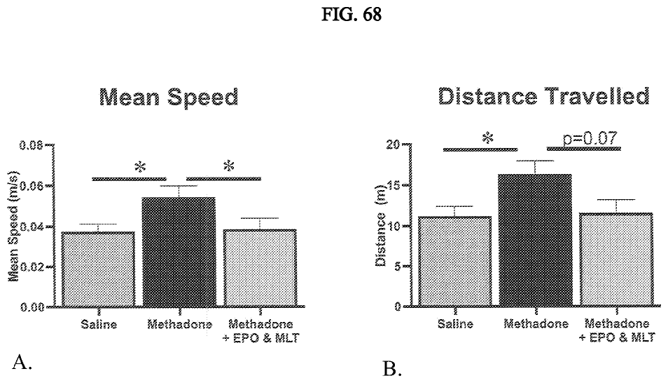

To mimic perinatal opioid exposure (POE), infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) or vehicle from P1-P10. For Open Field testing at P35, an individual rat is placed in a circular arena is divided into three concentric rings: wall, neutral zone and center. The activity of the rat is videotaped over 15 minutes and analyzed in three 5-minute intervals. Rats with POE treated with vehicle showed hyperactivity characterized by increased speed and distance traveled in the arena compared to saline controls, or EPO+MLT-treated rats with POE. (*p<0.05). Results are shown in FIG. 68 (includes FIGS. 68A-68B).

### Example 32: EPO+MLT Mitigates Specific Gait Abnormalities In Adult Rats Exposed to Methadone In Utero

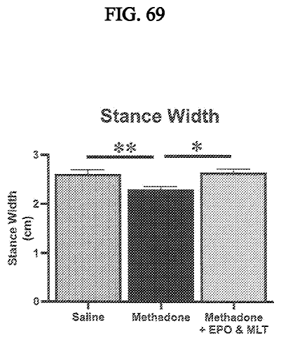

To mimic perinatal opioid exposure (POE), infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) or vehicle from P1-P10. Gait was assessed at P30 using digital analyses of videotaped treadmill performance. Rats with POE exhibited a narrower stance width, associated with a more unstable gait, compared to saline controls or rats with POE treated with EPO+MLT. These data show that neonatal EPO+MLT treatment can normalize neurological deficits in rats exposed to perinatal opioids, including abnormal motor development. (*p<0.05, **p<0.01). Results are shown in FIG. 69.

### Example 33—EPO+MLT Mitigates Allodynia and Thermal Hypersensitivity in Adult Rats Exposed to Methadone In Utero

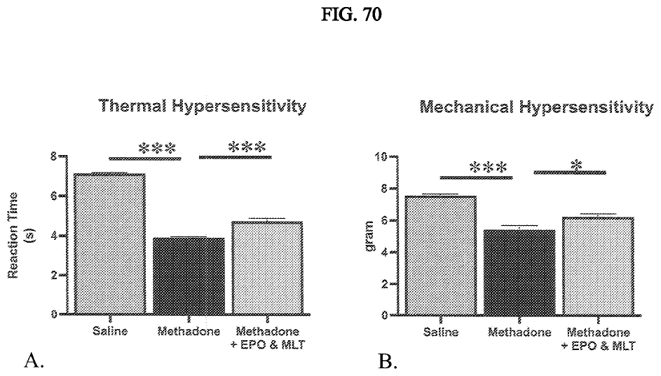

To mimic perinatal opioid exposure, infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) or vehicle from P1-P10. At P60 rats with POE exhibit more sensitivity to noxious thermal stimulation (tail immersion at 48° C.) compared to saline controls, or rats with POE treated with EPO+MLT. Similarly, rats with POE are also more sensitive to non-noxious mechanical allodynia (von Frey filaments with up-down method) compared to saline controls, or rats with POE treated with EPO+MLT. (*p<0.05, ***p<0.001). Results are depicted in FIG. 70 (includes FIGS. 70A-70B).

### Example 34—EPO+MLT Attenuates Blood Biomarkers of Inflammation in Adult Rats with Prenatal Methadone Exposure

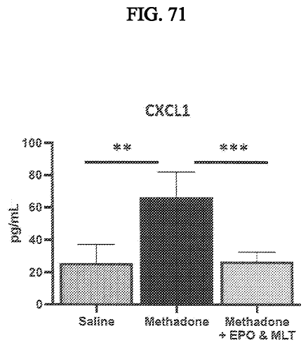

EPO+MLT Attenuates Blood Biomarkers of Inflammation in Adult Rats with Prenatal Methadone Exposure. To mimic perinatal opioid exposure, infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) from P1-P10. Serum was collected at P90 and assayed using multiplex electrochemiluminescence, an established platform for clinical biomarker quantification. In rats with POE, a marked increase in CXCL1 (chemokine CXC-ligand 1) is present, compared to saline controls or rats with POE treated with EPO+MLT. (**p<0.01). Results are shown in FIG. 71.

### Example 35—EPO+MLT Ameliorates Deficits in Structural Connectivity in Adult Rats Exposed to Methadone In Utero

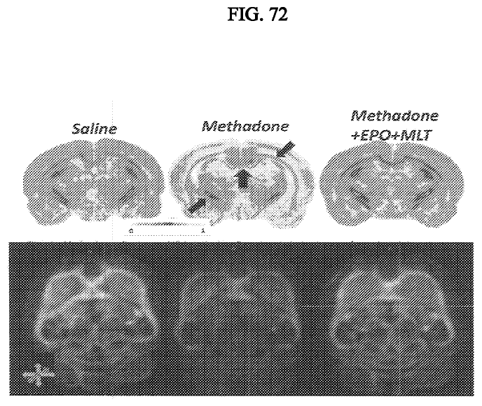

EPO+MLT Ameliorates Deficits in Structural Connectivity in Adult Rats Exposed to Methadone In Utero. To mimic perinatal opioid exposure, infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) from P1-P10. At ˜P90 adult rats underwent in vivo MRI followed by Diffusion Tensor Imaging analyses. The top images show loss of white matter microstructural integrity using Fractional Anisotropy (arrows, less blue). Bottom images show directional color-coded analyses. Note the brighter, more defined tracts in the saline controls and EPO+MLT-treated rats with POE, compared to muted tracts in rats with POE. Results are shown in FIG. 72.

### Example 36—EPO+MLT Ameliorates Deficits in Structural Connectivity in Adult Rats Exposed to Methadone

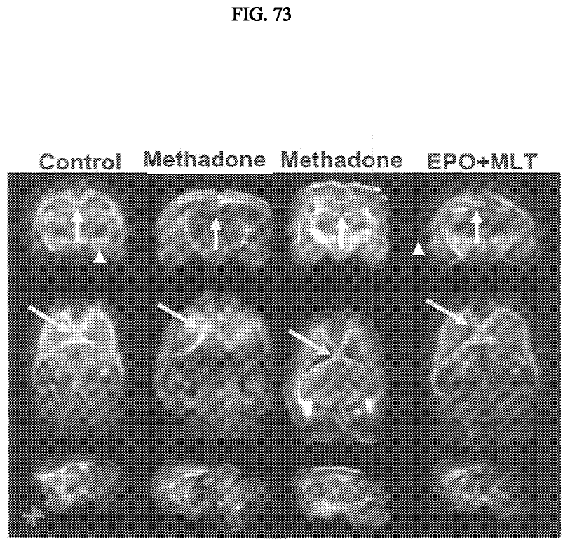

In Utero. To mimic perinatal opioid exposure, infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) from P1-P10. At ˜P90 adult rats underwent in vivo MRI followed by Diffusion Tensor Imaging analyses. Images (FIG. 73) show directional color-coded analyses (yellow transverse, red vertical, green orthogonal). Note the brighter, more defined tracts in the saline controls and EPO+MLT-treated rats with POE, compared to muted tracts in rats with POE (white arrows). (n=10-15, **p<0.01). Results are shown in FIG. 73.

### Example 37—EPO+MLT Ameliorates Deficits in Fractional Anisotropy (FA) in Adult Rats after Methadone Exposure, Consistent with Improved White Matter Health and Structure

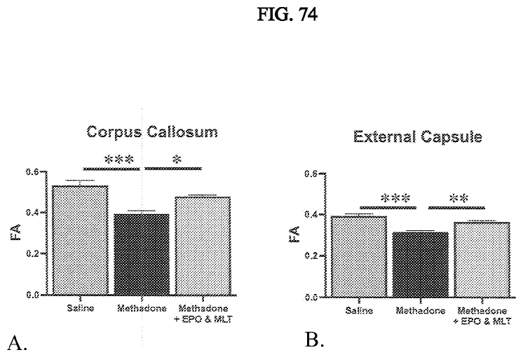

To mimic perinatal opioid exposure, infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) from P1-P10. At ˜P90 adult rats underwent in vivo MRI followed by Diffusion Tensor Imaging analyses. Fractional Anisotropy (FA) region of interest (ROI) were quantified by observers masked to injury and treatment group. Major white matter tracts in rats with POE showed significant loss of FA, consistent with loss of white matter microstructural integrity compared to saline controls, or rats with POE treated with EPO+MLT. (*p,0.05, **p<0.01, ***p<0.001). Results are shown in FIG. 74 (includes FIGS. 74A-74B).

### Example 38—EPO+MLT Normalizes Functional Activation in the Striatum of Adult Animals after Exposure to Methadone in Utero

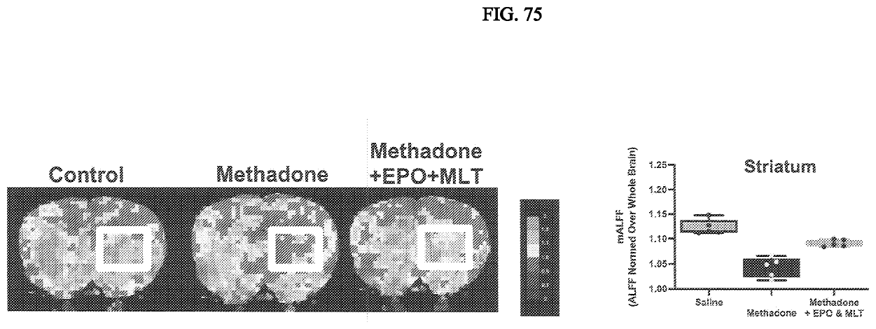

EPO+MLT Normalizes Functional Activation in the Striatum of Adult Animals after Exposure to Methadone In Utero. To mimic perinatal opioid exposure, infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) or vehicle from P1-P10. At ˜P90 adult rats underwent in vivo MRI followed data analyses for functional activation. The mean amplitude of low frequency fluctuations (mALFF) reflects neural activity. The mALFF was quantified in regions by observers blinded to treatment group. Coronal images on left show color-coded mALFF, with the striatum outlined by white box. Vehicle-treated rats with POE have lower mALFF in the striatum compared to EPO+MLT-treated rats, or saline controls. Reduced neural activity in the striatum is consistent with significant cognitive impairment observed in rats with POE. Results are shown in FIG. 75.

### Example 39—EPO+MLT Normalizes Functional Activation in the Amygdala of Adult Animals after Exposure to Methadone In Utero

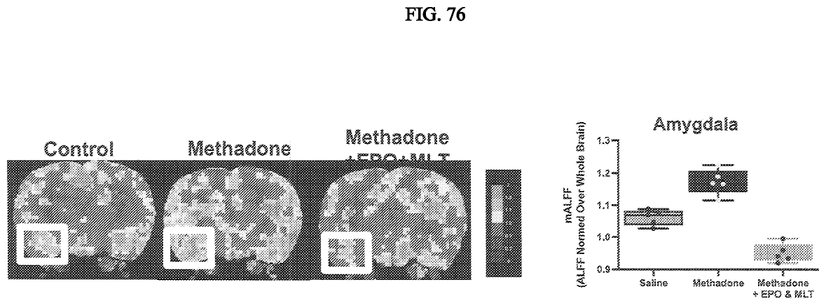

To mimic perinatal opioid exposure, infusion pumps with methadone or sterile saline were implanted in pregnant dams at embryonic day 16, equivalent to second trimester in humans. After birth, pups continued to receive methadone or saline via maternal milk until weaning on postnatal day 21 (P21). Methadone-exposed pups were randomized to receive neonatal erythropoietin (EPO) plus melatonin (MLT) or vehicle from P1-P10. At ˜P90 adult rats underwent in vivo MRI followed data analyses for functional activation. The mean amplitude of low frequency fluctuations (mALFF) reflects neural activity. The mALFF was quantified in regions by observers blinded to treatment group. Coronal images on left show color-coded mALFF, with the amygdala outlined by white box. Vehicle-treated rats with POE have higher mALFF in the amygdala compared to EPO+MLT-treated rats, or saline controls. Results are shown in FIG. 76. Increased neural activity in the amygdala is consistent with significant alterations in behavior including hyperactivity (FIG. 68) and lack of motor inhibition (FIG. 67) shown on open field and altered pain phenotype (FIG. 70).

### Example 40: Preterm Neonates with Severe Intraventricular Hemorrhage (SCEMPI)

The following is a clinical study of very preterm neonates with sIVH.

Subjects who are between 23w0d and 31w6/7d PMA and have initial sIVH on HUS within DOL21 and within past 5 days will be screened. PMA will be determined by the following hierarchy: gestational age from in vitro fertilization, first trimester assessment (0-14w0d), second trimester assessment, last menstrual period, newborn maturational assessment. Outborn neonates will be eligible. Screening logs will be kept to evaluate recruitment. Eligibility will be confirmed by research physician who obtains consent. Inclusion Criteria are 1) born between 23w0d and 31w6/7d PMA and 12 h-21 days old 2) sIVH, at least unilateral grade 1111 within past 5 days, 3) expected to survive >3 days, 4) absence of a congenital anomaly of metabolic or genetic disorder with expected survival <term equivalent, 5) approval of primary neonatologist, 6) arterial or venous access, 7) appropriate caregiver to provide informed consent.

Exclusion criteria: 1) life expectancy <3 days, 2) severe congenital anomaly or genetic disorder with life expectancy <40 w PMA, 3) severe liver failure, 4) active hematologic crisis such as disseminated intravascular coagulation 5) hydrops fetalis, 6) polycythemia (hematocrit >65%), 7) thrombus, 8) clinical concern or diagnosis of toxoplasmosis, cytomegalovirus, rubella or syphilis, 9) no appropriate person available or willing to provide informed consent.

Study Procedures: Subjects will undergo screening, informed consent, enrollment, baseline data collection, and will be randomized as described below to either MLT and EPO treatment, or placebo.

1. Screening and Informed Consent: Screening log will be completed for all NICU neonates between 23w0d and 31w6/7d PMA and who suffer sIVH (at least unilateral grade III) before DOL21, and within past 5 days. Research nurses will document reasons for ineligibility and confirm with primary neonatologist and research physicians. Informed consent will typically be obtained from biological mother, including emancipated minors. If biological mother is not available, then the social worker will identify if any other persons may provide consent.

2. IRB: IRB approval will be obtained and followed for all study procedures. An Investigational New Drug (IND) application will likely be required, we will submit an IND application with Dr. Robinson as the sponsor.

3. Baseline Data Collection: Baseline data will include clinical parameters including vital signs, growth parameters, presence of any pre-existing co-morbidities, and laboratory studies. HUS and baseline blood and urine samples will be obtained within 24 hours prior to initiating study drug.

4. Dosing Strategy: Three subjects will complete drug treatment at 33w6/7d, and be observed for one month, 37w6/7d. If the interim safety analysis shows it is safe to proceed, then 6 more subjects will be enrolled, randomized (3:1) to treatment with MLT 20 mg/kg/dose plus EPO, or placebo. Acute toxicity evaluation period will be 30 days. If after interim safety analysis the DSMB recommends proceeding, then two successive cohorts of 12 more subjects will be enrolled, randomized, treated and observed. If the interim safety analysis shows the rate of death and SAE for those treated with MLT 20 mg/kg/dose plus EPO does not differ from placebo with SOC, then additional cohorts of 12 will be randomized to treatment with MLT 30 mg/kg/dose plus EPO versus placebo until up to 60 subjects have completed treatment and observation.

5. Assessments: Clinical assessments will be performed daily (M-F) for primary and secondary outcomes. Research nurses will note any serious SAE and initiate appropriate notification of study team members. Secondary outcomes, preterm co-morbidities are documented. As secondary outcomes are collected on all preterm infants as part of quality improvement, regardless of study enrollment, these secondary outcomes will be collected as routine data. Weekly blood laboratory studies, including LFTs are obtained as part of SOC. Blood and urine samples will be collected at 5 time points for pharmacology studies and biomarkers. Surplus blood from routine clinical care will be used whenever possible. Serial weekly HUS and term equivalent neonatal brain MR are obtained as part of routine clinical care for all preterm infants with sIVH. Neonatal neurodevelopmental examinations will be obtained at 34 and 36 weeks, and include 1) NICU Network.

Neuroimaging biomarkers. HUS measurements of ventriculomegaly correlate with neurodevelopmental outcomes in preterm neonates with sIVH. Neuroradiologist Dr. Guryildirim and her team will assess weekly HUS for standard parameters such as anterior horn width and frontal occipital horn ratio.127 Neonatal brain MRI is performed as part of routine clinical care for neonates with sIVH near term and include T1, T2, SWI and diffusion tensor imaging. Images will be analyzed for structural parameters used in prior clinical trials of neonates with sIVH who develop PHH.

Neonatal neurodevelopmental examinations. Examinations will be obtained at 34 and 36 weeks PMA, and include NICU Network Neurobehavioral Scale (NNNS), Hammersmith Neonatal Neurological Examination (HNNE) and Prechtl's General Movement Assessment (GMA). The NNNS correlates with outcomes for preterm neonates with sIVH.

Liquid Biomarkers: Serum and urine will be obtained at 5 time points: 1) <24 hrs prior to study drugs, 2) day 7 at 30 min after 4th rhEPO dose (EPO peak), 3) day 9 at 30 min before 5th rhEPO dose (EPO trough), 4) 33w6d (±1d), when complete study drug, and 5) 36w6d(t2d) PMA, when complete monitoring. Serum levels are currently the standard liquid for biomarkers in this population. Urine will be compared to serum to determine if urine levels could substitute for serum levels in future trials. CSF is not routinely obtained in this population. Whenever possible, we will use surplus blood from labs obtained for routine clinical care. Research nurses will coordinate sample collection the bedside team and transfer samples to Dr. Jantzie's lab for separation, labeling, aliquoting and storage at −80° C. in a locked freezer. Samples will be assayed by observers blinded to other data.

MLT and EPO levels. MLT and levels in serum and urine will be quantified. EPO and MLT levels have not been previously assayed in preterm neonates with sIVH. We chose to collect levels at day 7 just after the 4th EPO dose (peak) and just prior to the 5th EPO dose (trough) to determine the EPO and MLT levels at a stable point in the dosing regimen. These time points were also used in the PENUT trial66, and using the same time points will facilitate comparison of EPO levels from this trial with trials. Here, EPO levels in serum and urine will be quantified using the mesoscale discovery (MSD) platform with an R-Plex plate, as in prior trials for preterm infants.68 Duplicates will be assayed using 50 μl total. MLT levels in serum and aMT6s levels in urine will be quantified by the JH research core using HPLC with tandem assays. Each assay will require 0.5 ml.

Cytokine and growth factor profile. Cytokines and chemokines IL-6, IL-8, IL-10, CCL2, CXCL1, and growth factors BDNF, EPO, VEGFA, NCAM-1, NGAL2 and GFAP will be assayed on a multiplex plate in duplicates (25 μl each). Additional aliquots of serum and urine will be stored to assay emerging biomarkers. Biomarker data from this trial will likely not provide definitive results but will provide initial data to inform future clinical trial design.

As systemic inflammation appears to have a significant role in the conversion from IVH to PHH, we will evaluate if MLT plus EPO impacts the inflammatory profile. Pro-inflammatory cytokines IL-6, IL-8, CCL2 and CXCL1 have been implicated in PBI, however their specific predictive value in preterm neonates with severe IVH is unclear.225 Similarly, anti-inflammatory cytokine IL-10 has also been implicated in PBI. We chose BDNF and VEGFA as potential biomarkers of brain health, and GFAP, NCAM-1 and NGL2 as markers of CNS damage. Emerging data in PBI suggest that resiliency of healthy brain may better predict the potential for recovery better than markers of injury.

Study Drug: Study drugs will consist of MLT and recombinant human EPO (rhEPO). The Research Pharmacy will randomize subjects to study drug or placebo, and supply and dispense study drugs. The research pharmacy team will not be blinded. They also will have no influence on the study assessments.

1. Dosage: MLT will be 20 or 30 mg/kg/dose enteral daily through 33w6/7d. MLT placebo will be same volume of sterile water. rhEPO will be 1000 U/kg/dose intravenous (IV) qod for 10 doses then 400 U/kg/dose subcutaneous (sc) qMWF through 33w6/7d. Placebo for rhEPO will be the same volume of sterile saline prepared for injection. Sham administration of placebo will be allowed to minimize needlesticks for se placebo as long as all other parties remain blinded.

2. Formulation: MLT will be enteral. The MLT regimen is 20 or 30 mg/kg/dose per day in a 10 mg/ml solution. For infants<1500 g, the dose will be given in two portions separated by 30 min. rhEPO can be given IV or sc.

3. Study Drug administration: Study drug will be provided in a syringe for enteral dosing for MLT and its placebo, and in a syringe for injection for rhEPO or its placebo.

4. Concomitant medications: All subjects will receive iron supplementation per the protocol used in the PENUT trial. Iron deficiency is common in very preterm babies and impairs CNS development. Enteral iron will be started when 1 week old AND tolerating enteral feeds of 60 mL/kg/day, 3 mg/kg/d when taking enteral feeds of 60-99 mL/kg/d, and 6 mg/kg/d when taking enteral feeds of 100-120 mL/kg/d. If not able to tolerate enteral iron, then will receive iron 3 mg/kg/wk IV. Serum ferritin or ratio of zinc protoporphyrin to heme will be assayed on DOL14 and 42. Neonates on study will not receive IV tissue plasminogen activator.

### Example 41: Treatment Protocol for a Subject Suffering from Long COVID-19 (Post-Acute Sequelae of COVID-19 (PASC))

A human patient is selected for treatment after being identified as suffering from symptoms of post-acute sequelae of COVID-19 (PASC), particularly one, two or more symptoms of COVID-19 for at least four weeks after initially testing positive for COVID-19.

The subject is treated with the following induction followed by maintenance treatment. The induction is treatment every 24 hours for 10 days with the following dosages of roxadustat and melatonin: 5 mg/kg/day of roxadustat (ROX) daily and 20 mg/kg/day melatonin (MLT) qhs. Maintenance regimen is Roxadustat 5 mg/kg/dose and 20 mg/kg/dose melatonin every other day (MWF) every other week.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method for treating a subject suffering from or susceptible to a condition associated with perinatal opioid exposure, the method comprising:
administering to a subject in need thereof an effective amount of one or more melatonin agents.

2. The method of claim 1 further comprising administering an effective amount of an erythropoietin agent to the subject.

3. The method of claim 1 is administered wherein the melatonin agent and erythropoietin agent are administered in combination to the subject.

4. The method of claim 1 further comprising administering an effective amount of a prolyl hydroxylase domain inhibitor (PHD) compound to the subject.

5. The method of claim 4 wherein the melatonin agent and prolyl hydroxylase domain inhibitor (PHD) compound are administered in combination to the subject.

6. The method of claim 4 wherein the 1) melatonin agent, 2) prolyl hydroxylase domain inhibitor (PHD) compound and 3) erythropoietin agent are administered in combination to the subject.

7. The method of claim 1 wherein the subject is pregnant.

8. The method of claim 7 wherein the subject or the subject's mother ingests one or more opioid compounds during pregnancy

9. The method of claim 1 wherein the subject is an infant.

10. The method of claim 1 wherein the melatonin agent is melatonin.

11. The method of claim 1 wherein the erythropoietin agent is human recombinant erythropoietin.

12. The method of claim 1 wherein the prolyl hydroxylase domain inhibitor (PHD) compound is TZ-951; FG4592 (Roxadustat); GSK1278863 (Daprodustat); Bay85-3934 (Molidustat); adaptaquin; or AKB-6548 (Vadadustat).

13. The method of claim 1 wherein the subject has been identified as an opiate user or having ingested an opiate and thereby selected for treatment.

14. The method of claim 1 wherein the subject has perinatal opioid exposure.

15. The method of claim 1 wherein the subject is suffering from or is susceptible to a perinatal brain injury.

16. The method of claim 1 wherein the subject is suffering from posthemorrhagic hydrocephalus, postinfectious hydrocephalus; post-traumatic hydrocephalus.

17. A method for treating a subject suffering from a brain injury or disease, or neurodegenerative disorder or disease the method comprising:
administering to a subject in need thereof an effective amount of a combination of 1) one or more melatonin agents and 2) one or more propyl hydroxylase domain inhibitor (PHD) compounds.

18-24. (canceled)

25. A method for treating a subject having a COVID-19 infection, exhibiting symptoms of a COVID-19 infection, having suspected exposure to COVID-19, or suspected of suffering from long COVID or post-acute sequelae of COVID-19 (PASC), the method comprising:
administering to a subject in need thereof an effective amount of a combination of 1) one or more melatonin agents and 2) one or more prolyl hydroxylase domain inhibitor (PHD) compounds, or
A method for treating a subject having a COVID-19 infection, exhibiting symptoms of a COVID-19 infection, having suspected exposure to COVID-19, or suspected of suffering from long COVID or past-acyte sequelae of COVID-19 (PASC), the method comprising:
administering to a subject in need thereof an effective amount of a combination of 1) one or more melatonin agents and 2) one or more erythropoietin agents

26-34. (canceled)

35. A pharmaceutical composition comprising:
(a) an effective amount of a melatonin agent; and
(b) an effective amount of an erythropoietin agent, or
A pharmaceutical composition comprising:
(a) an effective amount of a melatonin agent; and
(b) an effective amount of a prolyl hydroxylase domain inhibitor (PHD) compound.

36-40. (canceled)

41. A treatment kit comprising:
(a) an effective amount of a melatonin agent;
(b) an effective amount of an erythropoietin agent; and
(c) instructions for use of the melatonin agent and erythropoietin agent, or
A treatment kit comprising:
(a) an effective amount of a melatonin agent;
(b) an effective amount of a prolyl hydroxylase domain inhibitor (PHD) compound; and
(c) instructions for use of the melatonin agent and the prolyl hydroxylase domain inhibitor (PHD) compound, or
A treatment kit comprising:
(a) an effective amount of a melatonin agent;
(b) instructions for use of the melatonin agent for treatment of a condition associated with perinatal opioid exposure.

42-44. (canceled)
